(19) United States
(12) Patent Application Publication
(10) Pub. No.: US 2022/0282275 A1
(43) Pub. Date: Sep. 8, 2022

(54) G-TO-T BASE EDITORS AND USES THEREOF

(71) Applicants: The Broad Institute, Inc., Cambridge, MA (US); President and Fellows of Harvard College, Cambridge, MA (US)

(72) Inventors: David R. Liu, Cambridge, MA (US); Kevin Tianmeng Zhao, Cambridge, MA (US); Michelle Richter, Cambridge, MA (US)

(73) Assignees: The Broad Institute, Inc., Cambridge, MA (US); President and Fellows of Harvard College, Cambridge, MA (US)

(21) Appl. No.: 17/294,287

(22) PCT Filed: Nov. 15, 2019
(86) PCT No.: PCT/US2019/061685

§ 371 Date: May 14, 2021

Related U.S. Application Data
(60) Provisional application No. 62/768,062, filed on Nov. 15, 2018.

(51) Int. Cl. C12N 15/85; C12N 9/02; C12N 9/10; C12N 15/11; A61K 48/00

(52) U.S. Cl. CPC C12N 15/85; C12N 9/0093; C12Y 117/03002; C12N 9/1007; C12Y 201/01032; C12N 15/11; A61K 48/0066; C07K 2319/80; C12N 2310/20; C12N 2310/3513

(57) ABSTRACT

The present disclosure provides for base editors which satisfy a need in the art for installation of targeted transversions of guanine (G) to thymine (T), or correspondingly, transversions of adenine (A) to cytosine (C). The domains of the disclosed base editors include a nucleic acid programmable DNA binding protein and a guanine oxidase or a guanine methyltransferase. The base editors may be engineered through the use of continuous or non-continuous evolution systems. In particular, the present disclosure provides for guanine-to-thymine (or cytosine-to-adenine) base editors that can install single-base trans version mutations. In addition, methods for targeted nucleic acid editing are provided. Further provided are pharmaceutical compositions comprising, and vectors and kits useful for the generation of, guanine-to-thymine base editors. Cells containing such vectors and cells containing base editors and guide RNAs are also provided. Further provided are methods of treatment comprising administering the base editors to a subject in need thereof.

### RELATED APPLICATIONS

This application claims priority to U.S. Provisional Patent Application No. 62/768,062, filed Nov. 15, 2018, which is incorporated herein by reference.

### BACKGROUND OF THE INVENTION

Targeted editing of nucleic acid sequences, including the targeted cleavage or targeted introduction of a specific modification into genomic DNA, is a highly promising approach for the study of gene function and also has the potential to provide new therapies for human genetic diseases, including those caused by point mutations. Point mutations represent the majority of known human genetic variants associated with disease. Developing robust methods to introduce and correct point mutations is therefore an important challenge in understanding and treating diseases with a genetic component.

Base editing involves the conversion of a specific nucleic acid base into another at a targeted genomic locus. For certain approaches, this can be achieved without requiring double-stranded DNA breaks (DSB). Engineered base editors are capable of editing many targets with high efficiency, often achieving editing of 30-70% of cells following a single treatment, without selective enrichment of the cell population for editing events.

### SUMMARY OF THE INVENTION

Engineered base editors have been recently developed. Reference is made to Komor, A. C. et al., Improved base excision repair inhibition and bacteriophage Mu Gam protein yields C:G-to-T:A base editors with higher efficiency and product purity, Sci Adv 3 (2017) and Rees, H. A. et al., Improving the DNA specificity and applicability of base editing through protein engineering and protein delivery, Nat. Comun. 8, 15790 (2017); U.S. Patent Publication No. 2018/0073012, published Mar. 15, 2018; U.S. Patent Publication No. 2017/0121693, published May 4, 2017; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, each of which is incorporated herein by reference.

Base editors (BEs) are typically fusions of a Cas (“CRISPR-associated”) domain and a nucleobase modification domain (e.g., a natural or evolved deaminase, such as a cytidine deaminase, e.g., APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

Two classes of base editors have been generally described to date: cytosine base editors convert target C:G base pairs to T:A base pairs, and adenosine base editors convert A:T base pairs to G:C base pairs. Collectively, these two classes of base editors enable the targeted installation of all possible transition mutations (C-to-T, G-to-A, A-to-G, T-to-C, C-to-U, and A-to-U), which collectively account for about 61% of known human pathogenic single nucleotide polymorphisms (SNPs) in the ClinVar database. See Gaudelli, N. M. et al., Programmable base editing of A:T to G:C in genomic DNA without DNA cleavage. Nature 551, 464-471 (2017), which is incorporated herein by reference. In particular, C-to-T base editors use a cytidine deaminase to convert cytidine to uridine in the single-stranded DNA loop created by the Cas9 (“CRISPR-associated protein 9”) domain. The opposite strand is nicked by Cas9 to stimulate DNA repair mechanisms that use the edited strand as a template, while a fused uracil glycosylase inhibitor slows excision of the edited base. Eventually, DNA repair leads to a C:G to T:A base pair conversion. This class of base editor is described in U.S. Patent Publication No. 2017/0121693, published May 4, 2017, which issued on Jan. 1, 2019 as U.S. Pat. No. 10,167,457, each of which is incorporated herein by reference.

A major limitation of base editing is the inability to generate transversion (purine↔pyrimidine) changes, which are needed to correct the remaining ˜38% of known human pathogenic SNPs. See Komor, A. C. et al., Programmable editing of a target base in genomic DNA without double-stranded DNA cleavage, Nature 533, 420-424 (2016); and Landrum, M. J. et al., ClinVar: public archive of relationships among sequence variation and human phenotype, Nucleic Acids Res. 42, D980-985 (2014), each of which is incorporated herein by reference. Of this ˜38% of known pathogenic SNPs, about 15% arise from C:G to A:T mutations. Many C:G to A:T point mutations introduce premature stop codons (UAA, UAG, UGA), resulting in nonsense mutations in protein coding regions.

Currently, transversions can only be repaired by nuclease-mediated formation of a double-stranded break (DSB) followed by homology directed repair (HDR), which is typically inefficient, especially in non-mitotic cells, and leads to undesired byproducts such as indels (insertions and deletions) and translocations. See Komor, A. C., Badran, A. H. & Liu, D. R. CRISPR-Based Technologies for the Manipulation of Eukaryotic Genomes, Cell 168, 20-36, (2017), herein incorporated by reference. Since nucleobase deamination alone cannot interconvert purines and pyrimidines, the development of transversion base editors requires the development of a new editing strategy, such as the manipulation of endogenous DNA repair pathways or a different nucleobase chemical transformation. The present invention describes the first transversion base editors using two innovative strategies. The present invention greatly expands the capabilities of base editing.

In particular, the present disclosure provides for guanine-to-thymine or “GTBE” (or cytosine-to-adenine or “CABE”) base editors which satisfy a need in the art for installation of targeted single-base transversion nucleobase changes in a target nucleotide sequence, e.g., a genome. In addition, the present disclosure provides for nucleic acid molecules encoding and/or expressing the CABE base editors described herein, as well as vectors or constructs for expressing the CABE base editors described herein, host cells comprising said nucleic acid molecules and expression vectors, and compositions for delivering and/or administering nucleic acid-based embodiments described herein. In addition, the disclosure provides for CABE base editors, as well as compositions comprising the CABE base editors as described herein. Still further, the present disclosure provides for methods of making the CABE base editors, as well as methods of using the CABE base editors or nucleic acid molecules encoding the CABE base editors in applications including editing a nucleic acid molecule, e.g., a genome. This new strategy allows for the efficient and specific transversion of G-to-T or C-to-A using the base editors described herein. Two approaches are disclosed to achieve this specific transversion: the oxidation approach and the alkylation approach.

In the oxidation approach, enzyme-catalyzed guanine oxidation is induced at a targeted G in a DNA of interest, resulting in 8-oxoguanine (8-oxo-G) formation (FIG. 1A). 8-oxo-G occurs naturally and induces steric rotation of the damaged G around the glycosidic bond, forcing base pairing in the Hoogsteen orientation of 8-oxo-G. Without being bound by theory, the cell recognizes the mismatch between 8-oxo-G and the cytosine on the unmutated strand and repairs the cytosine to an adenine. Upon a subsequent round of replication or mismatch repair, the 8-oxo-G is converted to a thymine (see FIG. 2A). A desired G-to-T transversion is thus achieved. Guanine oxidation is achieved by the targeted application of a fusion protein comprising a dCas9 or nCas9 domain, an evolved guanine oxidase domain and a peptide linker connecting these two domains.

Targeted guanine oxidation is achieved by the use of a fusion protein comprising a nucleic acid programmable DNA binding protein domain, a guanine oxidase domain, and optionally a linker connecting these two domains (see FIG. 1A). The napDNAbp domain may be a catalytically dead Cas9 (“dCas9”) or Cas9 nickase (“nCas9”).

In the alkylation approach, enzyme-catalyzed methylation of a targeted G in a DNA of interest is induced, resulting in N₂,N₂-dimethyl-guanine or N₁-methyl-guanine formation (FIG. 1B). Both N₂,N₂-dimethyl-guanine and N₁-methyl-guanine disrupt the hydrogen bonding interactions with the cytosine of the unmutated strand. Without being bound by theory, the cell's replication machinery interprets the mutated guanine as a thymine, and converts the mismatched cytosine to an adenine. During a subsequent round of replication or mismatch repair, the alkylated guanine is converted to a thymine (see FIG. 2B). A desired G-to-T transversion is thus achieved. Guanine alkylation is achieved by the targeted application of a fusion protein comprising a dCas9 or nCas9 domain, an evolved guanine methyltransferase domain and a linker connecting these two domains.

The linker fusing the napDNAbp and guanine oxidase (or guanine methyltransferase) may be any suitable amino acid linker sequence, polymer, or covalent bond. Exemplary linkers include any of the following amino acid sequences:

(SEQ ID NO: 11)
SGGSSGGSSGSETPGTSESATPESSGGSSGGS;
(SEQ ID NO: 12)
SGGSGGSGGS;
(SEQ ID NO: 1)
GGG; GGGS;
(SEQ ID NO: 2)
SGGGS;
(SEQ ID NO: 48)
SGSETPGTSESATPES;
or
(SEQ ID NO: 14)
SGGS.

Accordingly, in some aspects, the base editor comprises (i) a nucleic acid programmable DNA binding protein (napDNAbp) domain and (ii) a guanine oxidase domain. The napDNAbp domain may comprise a Cas9 domain. The napDNAbp domain may be a CasX (Cas12e), CasY (Cas12d), Cpf1, C2c1, C2c2 (Cas13a), C2c3 (Cas12c), GeoCas9, CjCas9, Cas12a, Cas12b, Cas12g, Cas12h, Cas12i, Cas13b, Cas13c, Cas13d, Cas14, Csn2, or Argonaute (Ago) protein. The napDNAbp domain may be a nuclease active Cas9 domain, a nuclease inactive Cas9 (dCas9) domain, or a Cas9 nickase (nCas9) domain. The napDNAbp domain may be a Cas9 domain derived from S. pyogenes, or an SpCas9.

In various embodiments of the base editors, the guanine oxidase is a wild-type guanine oxidase, or a variant thereof, that oxidizes a guanine in DNA. In certain embodiments, the guanine oxidase is a xanthine dehydrogenase, or a variant thereof. In certain embodiments, the xanthine dehydrogenase is a Streptomyces cyanogenus xanthine dehydrogenase (ScXDH) or variant thereof. In other embodiments, the xanthine dehydrogenase or variant thereof is derived from C. capitata, N. crassa, M. hansupus, E. cloacae, S. snoursei, S. albulus, S. himastatinicus, or S. lividans.

In various embodiments, the base editor further comprises an 8-oxoguanine glycosylase (OGG or OGG1) inhibitor (“OGG inhibitor”) or catalytically inactive OGG1 enzyme.

In another aspect, the base editor comprises (i) a nucleic acid programmable DNA binding protein (napDNAbp), and (ii) a guanine methyltransferase. In various embodiments of the base editors, the guanine methyltransferase is a wild-type guanine methyltransferase. In certain embodiments, the guanine methyltransferase is a wild-type RlmA, or a variant thereof, that methylates a guanine in DNA. In certain embodiments, the RlmA is an Escherichia coli RlmA, or a variant thereof.

In other embodiments, complexes comprising any of the fusion proteins described herein and a guide RNA bound to the napDNAbp domain of the fusion protein are provided.

In various embodiments, the disclosure provides nucleic acids and vectors encoding any of the base editors, or domains thereof, described herein. The nucleic acid sequences may be codon-optimized for expression in the cells of any organism of interest (e.g., human). In certain embodiments, the nucleic acid sequence is codon-optimized for expression in human cells.

In other embodiments, cells containing the nucleic acids, cells containing the vectors, and cells containing the complexes described herein are provided. Further provided are cells containing purified base editors, or domains thereof, as described herein.

In other embodiments, the disclosure provides a pharmaceutical composition comprising any of the fusion proteins described herein and a pharmaceutically acceptable excipient. In certain embodiments, the pharmaceutical composition further comprises a gRNA.

In other embodiments, the disclosure provides a kit comprising a nucleic acid construct that includes (i) a nucleic acid sequence encoding any of the fusion proteins described herein; (ii) a heterologous promoter that drives expression of the sequence of (i); and optionally an expression construct encoding a guide RNA backbone and the target sequence. The disclosure further provides kits comprising a fusion protein as provided herein, a gRNA having complementarity to a target sequence, and cofactor proteins, buffers, media, and/or target cells.

In some embodiments, methods for targeted nucleic acid editing are provided. The methods described herein typically comprise i) contacting a nucleic acid sequence with a complex comprising any of the fusion proteins described herein and a guide nucleic acid, wherein the nucleic acid is a double-stranded DNA comprises a target G:C (or C:G) nucleobase pair; and ii) editing the thymine (or adenine) of the G:C (or C:G) nucleobase pair. The methods may further comprise iii) cutting or nicking a strand of the double-stranded DNA (e.g., nicking the non-edited strand of the DNA).

In some embodiments, methods of treatment using the base editors described herein are provided. The methods described herein may comprise treating a subject having or at risk of developing a disease, disorder, or condition, comprising administering to the subject a fusion protein as described herein, a polynucleotide as described herein, a vector as described herein, or a pharmaceutical composition as described herein.

In various other embodiments, the specification provides nucleic acid molecules encoding any of the base editors, or domains thereof. The nucleic acid sequences may be codon-optimized for expression in mammalian cells. In certain embodiments, the nucleic acid sequence is optimized for expression in human cells.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain embodiments of the present disclosure, which can be better understood by reference to one or more of these drawings in combination with the detailed description of specific embodiments presented herein.

FIG. 1A is a schematic illustration showing an exemplary fusion protein of the invention. A fusion protein comprising a dCas9 domain linked to a guanine oxidase enzyme is targeted to the correct guanine nucleobase through the hybridization of a single-guide RNA (“sgRNA”) to a complementary sequence of nucleic acid. The guanine oxidase oxidizes the guanine to 8-oxo-G, and subsequently, the cell's native replication/repair machinery recognizes the mutated base and effectuates the desired change to a thymine nucleobase. Depicted here is the intermediate of the guanine oxidation reaction, in which guanine has been oxidized to 8-oxo-G following the creation of an R-loop (a DNA:RNA:DNA triplex structure) at the target base pair site by the dCas9 domain. Abbreviations: OGG, 8-oxoguanine glycosylase; 8OG, 8-oxo-guanine; sgRNA, single-guide RNA; PAM, protospacer adjacent motif.

FIG. 1B is a schematic illustration showing an exemplary fusion protein of the invention. A fusion protein comprising a dCas9 domain linked to a guanine methyltransferase is targeted to the correct guanine nucleobase through the hybridization of an sgRNA to a complementary sequence of DNA. The guanine methyltransferase methylates the guanine to N₂,N₂-dimethyl-guanine or N₁-methyl-guanine, and subsequently, the cell's native replication/repair machinery recognizes the altered base and effectuates the desired change from the C:G nucleobase pair to an A:T nucleobase pair. Depicted here is the intermediate of the guanine methylation reaction, in which guanine has been methylated to N₁-methyl-guanine following the creation of an R-loop at the target base pair site by the dCas9 domain. Abbreviations: ALRE, alkylation lesion repair enzyme; N₁MG, N₁-methyl guanine; sgRNA, single-guide RNA; PAM, protospacer adjacent motif.

FIG. 2A depicts a possible chemical mechanism for the conversion of guanine to thymine by one or more of the disclosed base editors. A guanine oxidase enzyme recognizes a target guanine base within a target sequence to which the sgRNA has complementarity. The enzyme mediates the oxidation of guanine to 8-oxo-guanine. Steric rotation of the 8-oxo-G around the glycosidic bond is induced, presenting the Hoogsteen edge for base pairing. Without wishing to be bound by any particular theory, during replication or repair of the unmutated strand, the 8-oxo-G is paired with cytosine by a DNA polymerase. The cell recognizes the mismatch between the 8-oxo-G and the cytosine on the unmutated strand and converts the cytosine to an adenine. Upon the next round of replication, the mutated guanine is converted to a thymine, thereby effecting a conversion from a G:C nucleobase pair to a A:T nucleobase pair. Abbreviation: MMR, mismatch repair.

FIG. 2B depicts a possible chemical mechanism for the conversion of guanine to thymine by one or more of the disclosed base editors. A guanine methyltransferase enzyme recognizes a target guanine base within a target sequence to which the sgRNA has complementarity. The enzyme mediates the methylation of guanine to N₂,N₂-dimethyl-guanine or N₁-methyl-guanine (e.g., an 8-methyl guanine). Steric rotation of the methylated guanine around the glycosidic bond is induced, presenting the Hoogsteen edge for base pairing. Without wishing to be bound by any particular theory, during replication or repair of the unmutated strand, the 8-methyl-guanine is paired with cytosine by a polymerase. The cell recognizes the mismatch between the methylated guanine and the cytosine on the unmutated strand and converts the cytosine to an adenine. Upon the next round of replication, the mutated guanine is converted to a thymine, thereby effecting a conversion from a G:C nucleobase pair to a A:T nucleobase pair. Abbreviation: MMR, mismatch repair.

FIG. 3 depicts an exemplary assay for selection of evolved variants of S. cyanogenus XDH that are effective at recognizing a (DNA) guanine base as a nucleobase substrate. Plasmids containing mutagenized ScXDH-dCas9 fusion proteins and targeting guide RNAs (sgRNAs), and selection plasmids containing an inactivated carbenicillin resistance gene with a premature stop codon (Y95X) or a mutation at the active site (S233A) that each require G:C-to-T:A editing to correct, are transformed into E. coli cells, which are plated onto agar media containing carbenicillin and sucrose. Cells harboring plasmids with ScXDH mutants that restore antibiotic resistance are isolated and subjected to further rounds of mutation and selection under varying selection stringencies. ScXDH variants emerging from each round of selection are then expressed within a fusion construct comprising a Cas9 nickase (nCas9). The resulting fusion proteins are tested for base editing activity in mammalian cells.

FIG. 4A depicts the chemical conversion of guanine to N₂,N₂-dimethyl guanine, which disrupts existing hydrogen bonding with the cytosine of the unmutated strand. The cell's replication machinery interprets the mutated guanine as a T, and converts the mismatched cytosine to an adenine. During a subsequent replication-and-repair cycle, the mutated guanine is converted to a T, completing the desired T:A mutation. FIG. 4B depicts the chemical conversion of guanine to N₁-methyl guanine, which disrupts existing hydrogen bonding with the cytosine of the unmutated strand. The cell's replication machinery interprets the mutated guanine as a T, and converts the mismatched cytosine to an adenine. During a subsequent replication-and-repair cycle, the mutated guanine is converted to a T, completing the desired T:A mutation. Abbreviation: MMR, mismatch repair.

FIG. 5 depicts a schematic representation of the biotin pull-down assay of transformed oligonucleotide fragments that are the product of in vitro ligation of shorter target DNA oligos with modified (methylated) bases. The modified N₂,N₂-dimethyl-guanine and N₁-methyl-guanine nucleobases, with the methyl groups bolded, are also depicted.

FIG. 6 depicts charts showing sequencing reads of transformed oligonucleotide fragments having modified (methylated) bases. Phusion U, Q5, and Taq polymerases were applied to the pulled-down strand to identify the potential mutagenic effect.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

The term “accessory plasmid,” as used herein within the context of a continuous evolution protocol for engineering of protein variants, refers to a plasmid comprising a gene required for the generation of infectious viral particles under the control of a conditional promoter. In the context of the continuous evolution of genes, transcription from the conditional promoter of the accessory plasmid is typically activated, directly or indirectly, by a function of the gene to be evolved. Accordingly, the accessory plasmid serves the function of conveying a competitive advantage to those viral vectors in a given population of viral vectors that carry a version of the gene to be evolved able to activate the conditional promoter or able to activate the conditional promoter more strongly than other versions of the gene to be evolved. In some embodiments, only viral vectors carrying an “activating” version of the gene to be evolved will be able to induce expression of the gene required to generate infectious viral particles in the host cell, and, thus, allow for packaging and propagation of the viral genome in the flow of host cells. Vectors carrying non-activating versions of the gene to be evolved, on the other hand, will not induce expression of the gene required to generate infectious viral vectors, and, thus, will not be packaged into viral particles that can infect fresh host cells. Exemplary accessory plasmids have been described, for example in U.S. Patent Pub. No. 2018/0087046, published on Mar. 29, 2018, which is incorporated by reference herein.

In various embodiments of the continuous evolution methods described herein, a first accessory plasmid may comprise gene III, which is required to produce infectious progeny phage, operably linked to a T7 promoter; and a second accessory plasmid may comprise a T7 RNA polymerase (“RNAP”) gene that is deactivated by a G to T mutation, which results in an early stop codon. This non-activating mutation may be positioned in, for instance, a glutamate (E) residue encoded by GAA within the polymerase gene. Any of the E90STOP mutation, E91STOP mutation, E167STOP mutation, E168STOP mutation, or combinations thereof, may be used as the non-activating mutation.

A third accessory plasmid may comprise a nucleotide encoding a dCas9 fused at the N terminus to the C-terminal half of a fast-splicing intein. An exemplary phage plasmid may comprise a nucleotide encoding a guanine oxidase fused at the C terminus to the N-terminal half of the fast-splicing intein.

The full-length base editor may be reconstituted from the two intein components. Successful replication of phage progeny would require the base editor to perform G to T transversion mutations in the T7 RNAP gene, allowing successful translation of full-length T7 RNAP and subsequent transcription of gene III. The nucleotide encoding a guide RNA targeting dCas9 to the appropriate sequence of T7RNAP may be located on any of these accessory plasmids. For instance, it may be located on the first accessory plasmid, i.e. the same accessory plasmid on which gene III is located. This accessory plasmid design emulates the PACE circuit of cytosine base editors, as disclosed in Thuronyi et al., Continuous evolution of base editors with expanded target compatibility and improved activity, Nat Biotechnol. 2019 Jul. 22, International Application No. PCT/US2019/37216, filed Jun. 14, 2019, and International Patent Publication WO 2019/023680, published Jan. 31, 2019, each of which are incorporated herein by reference.

“Base editing” refers to a genome editing technology that involves the conversion of a specific nucleic acid base into another at a targeted genomic locus. In certain embodiments, this can be achieved without requiring double-stranded DNA breaks (DSB). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g., typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), which is incorporated by reference herein.

In principle, there are 12 possible base-to-base changes that may occur via individual or sequential use of transition (i.e., a purine-to-purine change or pyrimidine-to-pyrimidine change) or transversion (i.e., a purine-to-pyrimidine or pyrimidine-to-purine) editors. These include:
Transition base editors:
   C-to-T base editor (or “CTBE”). This type of editor converts a C:G Watson-Crick nucleobase pair to a T:A Watson-Crick nucleobase pair. Because the corresponding Watson-Crick paired bases are also interchanged as a result of the conversion, this category of base editor may also be referred to as a G-to-A base editor (or “GABE”).
   A-to-G base editor (or “AGBE”). This type of editor converts a A:T Watson-Crick nucleobase pair to a G:C Watson-Crick nucleobase pair. Because the corresponding Watson-Crick paired bases are also interchanged as a result of the conversion, this category of base editor may also be referred to as a T-to-C base editor (or “TCBE”).
Transversion base editors:
   G-to-T base editor (or “GTBE”). This type of editor converts a G:C Watson-Crick nucleobase pair to a T:A Watson-Crick nucleobase pair. Because the corresponding Watson-Crick paired bases are also interchanged as a result of the conversion, this category of base editor may also be referred to as a C-to-A base editor (or “CABE”).
   C-to-G base editor (or “CGBE”). This type of editor converts a C:G Watson-Crick nucleobase pair to a G:C Watson-Crick nucleobase pair. Because the corresponding Watson-Crick paired bases are also interchanged as a result of the conversion, this category of base editor may also be referred to as a G-to-C base editor (or “GCBE”).
   A-to-T base editor (or “ATBE”). This type of editor converts a A:T Watson-Crick nucleobase pair to a T:A Watson-Crick nucleobase pair. Because the corresponding Watson-Crick paired bases are also interchanged as a result of the conversion, this category of base editor may also be referred to as a T-to-A base editor (or “TABE”).
   A-to-C base editor (or “ACBE”). This type of editor converts a A:T Watson-Crick nucleobase pair to a C:G Watson-Crick nucleobase pair. Because the corresponding Watson-Crick paired bases are also interchanged as a result of the conversion, this category of base editor may also be referred to as a T-to-G base editor (or “TGBE”).

The term “base editors (BEs)” as used herein, refers to the improved Cas-fusion proteins described herein. In some embodiments, the fusion protein comprises a nuclease-inactive Cas9 (dCas9) fused to a guanine oxidase which still binds a nucleic acid in a guide RNA-programmed manner via the formation of an R-loop but does not cleave the nucleic acid. For example, the dCas9 domain of the fusion protein may include a D10A an H840A mutation. In other embodiments, the fusion protein comprises a Cas9 nickase (nCas9) fused to a guanine oxidase. The nCas9 domain of the fusion protein may include a D10A or an H840A mutation (which renders the Cas9 domain capable of cleaving only one strand of a nucleic acid duplex), as described in PCT/US2016/058344, filed on Oct. 22, 2016, and published as WO 2017/070632 on Apr. 27, 2017), which is incorporated herein by reference. The DNA cleavage domain of S. pyogenes Cas9 includes two subdomains, the HNH nuclease subdomain and the RuvC1 subdomain. The HNH subdomain cleaves the strand complementary to the gRNA (the “targeted strand,” or the strand at which guanine oxidation or alkylation occurs), whereas the RuvC1 subdomain cleaves the non-complementary strand containing the PAM sequence (the “non-targeted strand”, or the strand at which guanine oxidation or alkylation does not occur). The RuvC1 nCas9 mutant D10A generates a nick on the targeted strand, while the HNH nCas9 mutant H840A generates a nick on the non-targeted strand (see Jinek et al., Science 337:816-821(2012); Qi et al., Cell 28; 152(5):1173-83 (2013))

In some embodiments, the fusion protein comprises a Cas9 nickase fused to a guanine oxidase, e.g., a guanine oxidase which converts a DNA base guanine to 8-oxo-G. The term “base editors” encompasses any base editor known or described in the art at the time of this filing as well as any base editor known or described in the art at the time of this filing or developed in the future. Reference is made to Rees & Liu, Base editing: precision chemistry on the genome and transcriptome of living cells, Nat Rev Genet. 2018; 19(12):770-788 and Koblan et al., Nat Biotechnol. 2018; 36(9):843-846; as well as U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018; U.S. Provisional Application No. 62/835,490, filed Apr. 17, 2019; U.S. Provisional Application No. 62/814,798, filed Mar. 6, 2019; U.S. Provisional Application No. 62/814,766, filed Mar. 6, 2019; International Application No. PCT/US2019/57956, filed Oct. 24, 2019; U.S. Provisional Application No. 62/814,796, filed Mar. 6, 2019; U.S. Provisional Application No. 62/814,800, filed Mar. 6, 2019; U.S. Provisional Application No. 62/814,793, filed Mar. 6, 2019; U.S. Provisional Application No. 62/858,958, filed Jun. 7, 2019; International Publication No. PCT/US2019/58678, filed Oct. 29, 2019; International Patent Publication No. PCT/US2019/47996, filed Aug. 23, 2019; U.S. Provisional Application No. 62/884,459, filed Aug. 8, 2019; U.S. Provisional Application No. 62/8887,307, filed Aug. 15, 2019, and International Publication No. PCT/US2019/49793, filed Sep. 5, 2019, the contents of each of which are incorporated herein by reference.

The term “Cas9” or “Cas9 nuclease” or “Cas9 domain” refers to a CRISPR associated protein 9, or variant thereof, and embraces any naturally occurring Cas9 from any organism, any naturally-occurring Cas9, any Cas9 homolog, ortholog, or paralog from any organism, and any variant of a Cas9, naturally-occurring or engineered. More broadly, a Cas9 protein or domain is a type of nucleic acid programmable D/RNA binding protein (napR/DNAbp),” or more specifically, a “nucleic acid programmable DNA binding protein (napDNAbp)”. The term Cas9 is not meant to be limiting and may be referred to as a “Cas9 or variant thereof.” Exemplary Cas9 proteins are described herein and also described in the art. The present disclosure is unlimited with regard to the particular Cas9 that is employed in the base editors of the invention.

In some embodiments, proteins comprising Cas9 or fragments thereof are referred to as “Cas9 variants.” A Cas9 variant shares homology to Cas9, or a fragment thereof. Cas9 variants include functional fragments of Cas9. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to a wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

As used herein, the term “dCas9” refers to a nuclease-inactive Cas9 or nuclease-dead Cas9, or a variant thereof, and embraces any naturally occurring dCas9 from any organism, any naturally-occurring dCas9 equivalent, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and methods for making dCas9 proteins are further described herein and/or are described in the art and are incorporated herein by reference.

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactives one of the two endonuclease activities of the Cas9. Any suitable mutation which inactivates one Cas9 endonuclease activity but leaves the other intact is contemplated, such as one of D10A or H840A mutations in the wild-type Cas9 amino acid sequence (e.g., SEQ ID NO: 9) may be used to form the nCas9. In various embodiments, the D10A mutation is used to form the nCas9.

The term “continuous evolution,” as used herein, refers to an evolution procedure, (e.g., PACE) in which a population of nucleic acids is subjected to multiple rounds of (a) replication, (b) mutation, and (c) selection to produce a desired evolved product, for example, a nucleic acid encoding a protein with a desired activity, wherein the multiple rounds can be performed without investigator interaction, and wherein the processes under (a)-(c) can be carried out simultaneously. Typically, the evolution procedure is carried out in vitro, for example, using cells in culture as host cells. In general, a continuous evolution process provided herein relies on a system in which a gene of interest is provided in a nucleic acid vector that undergoes a life-cycle including replication in a host cell and transfer to another host cell, wherein a critical component of the life-cycle is deactivated and reactivation of the component is dependent upon a desired mutation in the gene of interest. Reference is made to U.S. Patent Publication No. 2013/0345064, which published on Dec. 26, 2013, and issued as U.S. Pat. No. 9,394,537 on Jul. 19, 2016; U.S. Patent Publication No. 2016/0348096, which published on Dec. 1, 2016 and issued as U.S. Pat. No. 10,179,911 on Jan. 15, 2019; U.S. Patent Publication No. 2017/0233708, which published Aug. 17, 2017; U.S. Patent Publication No. 2017/0044520, which published on Feb. 16, 2017; International Application No. PCT/US2019/37216, filed Jun. 14, 2019; International Patent Publication WO 2019/023680, published Jan. 31, 2019, and International Patent Publication No. PCT/US2019/47996, filed Aug. 23, 2019, the contents of each of which are incorporated herein by reference in their entireties.

In some embodiments, the nucleic acid vector of the continuous evolution system that comprises the gene of interest is a viral vector, a microparticle, a nanoparticle, a lipid particle, or naked DNA (e.g., a mobilization plasmid). In some embodiments, transfer of the gene of interest from cell to cell is via infection, transfection, transduction, conjugation, or uptake of naked DNA, and efficiency of cell-to-cell transfer (e.g., transfer rate) is dependent on the activity of a product encoded by the gene of interest. For example, in some embodiments, the nucleic acid vector is a phage harboring the gene of interest, and the efficiency of phage transfer (via infection) is dependent on an activity of the gene of interest in that a protein required for the generation of phage particles (e.g., pIII for M13 phage) is expressed in the host cells only in the presence of the desired activity of the gene of interest. In another example, the nucleic acid vector is a retroviral vector, for example, a lentiviral or vesicular stomatitis virus vector harboring the gene of interest, and the efficiency of viral transfer from cell to cell is dependent on an activity of the gene of interest in that a protein required for the generation of viral particles (e.g., an envelope protein, such as VSV-g) is expressed in the host cells only in the presence of the desired activity of the gene of interest. In another example, the nucleic acid vector is a DNA vector, for example, in the form of a mobilizable plasmid DNA, comprising the gene of interest, that is transferred between bacterial host cells via conjugation, and the efficiency of conjugation-mediated transfer from cell to cell is dependent on the activity of the gene of interest in that a protein required for conjugation-mediated transfer (e.g., traA or traQ) is expressed in the host cells only in the presence of the desired activity of the gene of interest. Host cells contain F plasmid lacking one or both of those genes.

For example, some embodiments provide a continuous evolution system, in which a population of viral vectors comprising a gene of interest to be evolved replicates in a flow of host cells, e.g., a flow through a lagoon, wherein the viral vectors are deficient in a gene encoding a protein that is essential for the generation of infectious viral particles, and wherein that gene is comprised in the host cell under the control of a conditional promoter that can be activated by a gene product encoded by the gene of interest, or a mutated version thereof. In some embodiments, the activity of the conditional promoter depends on a desired function of a gene product encoded by the gene of interest. Viral vectors, in which the gene of interest has not acquired a mutation conferring the desired function, will not activate the conditional promoter, or only achieve minimal activation, while any mutation in the gene of interest that confers the desired mutation will result in activation of the conditional promoter. Since the conditional promoter controls an essential protein for the viral life cycle, activation of this promoter directly corresponds to an advantage in viral spread and replication for those vectors that have acquired an advantageous mutation.

“CRISPR” is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively constitute, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate embodiments of both the crRNA and tracrRNA into a single RNA species—the guide RNA. See, e.g., Jinek M., et al., Science 337:816-821 (2012), the entire contents of which is incorporated herein by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti J. J., et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607 (2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes, S. thermophiles, C. ulcerans, S. diphtheria, S. syrphidicola, P. intermedia, S. taiwanense, S. iniae, B. baltica, P. torquis, S. thermophiles, L. innocua, C. jejuni, and N. meningitidis. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

The term “effective amount,” as used herein, refers to an amount of a biologically active agent that is sufficient to elicit a desired biological response. For example, in some embodiments, an effective amount of a base editor may refer to the amount of the base editor that is sufficient to edit a target site nucleotide sequence, e.g., a genome. In some embodiments, an effective amount of a base editor provided herein, e.g., of a fusion protein comprising a nuclease-inactive Cas9 domain and a nucleobase modification domain (e.g., a guanine oxidase domain) may refer to the amount of the fusion protein that is sufficient to induce editing of a target site specifically bound and edited by the fusion protein. In some embodiments, an effective amount of a base editor provided herein may refer to the amount of the fusion protein sufficient to induce editing having the following characteristics: >50% product purity, <5% indels, and/or an editing window of 2-8 nucleotides. In other embodiments, an effective amount of a base editor may refer to the amount of the fusion protein sufficient to induce editing of >45% product purity, <10% indels, a ratio of intended point mutations to indels that is at least 5:1, and/or an editing window of 2-10 nucleotides. U.S. Provisional Application No. 62/835,490, filed Apr. 17, 2019, is incorporated herein by reference. As will be appreciated by the skilled artisan, the effective amount of an agent, e.g., a fusion protein, a nuclease, a guanine oxidase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the target cell or tissue (i.e., the cell or tissue to be edited), and on the agent being used.

The term “evolved base editor” or “evolved base editor variant” refers to a base editor formed as a result of mutagenizing a reference base editor. The term also refers to embodiments in which the nucleobase modification domain is evolved or a separate domain is evolved. Mutagenizing a reference base editor may comprise mutagenizing a guanine oxidase or a guanine methyltranferase—by a continuous evolution method (e.g., PACE), wherein the evolved guanine oxidase or guanine methyltranferase has one or more amino acid variations introduced into its amino acid sequence relative to the amino acid sequence of the guanine oxidase or a guanine methyltranferase. Amino acid sequence variations may include one or more mutated residues within the amino acid sequence of a reference base editor, e.g., as a result of a change in the nucleotide sequence encoding the base editor that results in a change in the codon at any particular position in the coding sequence, the deletion of one or more amino acids (e.g., a truncated protein), the insertion of one or more amino acids, or any combination of the foregoing. The evolved base editor may include variants in one or more components or domains of the base editor (e.g., variants introduced into a guanine oxidase domain, a guanine methyltranferase domain, a 8-oxoguanine glycosylase (OGG) inhibitor, or ALRE inhibitor domain, or variants introduced into combinations of these domains).

The term “fusion protein,” as used herein, refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “host cell,” as used herein, refers to a cell that can host, replicate, and transfer a phage vector useful for a continuous evolution process as provided herein. In embodiments where the vector is a viral vector, a suitable host cell is a cell that can be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect. In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, and XL1-Blue MRF′. These strain names are art recognized and the genotype of these strains has been well characterized. It should be understood that the above strains are exemplary only and that the invention is not limited in this respect. The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

In some PACE embodiments, for example, in embodiments employing an M13 selection phage, the host cells are E. coli cells expressing the Fertility factor, also commonly referred to as the F factor, sex factor, or F-plasmid. The F-factor is a bacterial DNA sequence that allows a bacterium to produce a sex pilus necessary for conjugation and is essential for the infection of E. coli cells with certain phage, for example, with M13 phage. For example, in some embodiments, the host cells for M13-PACE are of the genotype F′proA⁺B⁺Δ(lacIZY) zzf::Tn10(TetR)/endA1 recA 1 galE15 galK16 nupG rpsL ΔlacIZYA araD139 Δ(ara,leu)7697 mcrA Δ(mrr-hsdRMS-mcrBC) proBA::pir116λ⁻.

The term “linker,” as used herein, refers to a chemical group or a molecule linking two molecules or domains, e.g., nCas9 and a guanine methyltransferase or guanine oxidase. In some embodiments, a linker joins a dCas9 and modification domain (e.g., a guanine oxidase). Typically, the linker is positioned between, or flanked by, two groups, molecules, or other domains and connected to each one via a covalent bond, thus connecting the two. In some embodiments, the linker is an amino acid or a plurality of amino acids (e.g., a peptide or protein). In some embodiments, the linker is an organic molecule, group, polymer, or chemical domain. Chemical domains include, but are not limited to, amide, urea, carbamate, carbonate, an ester, acetal, ketal, phosphoramidite, hydrazone, imine, oxime, disulfide, silyl, hydrazine, hydrazone, thiol, imidazole, carbon-carbon bond, carbon-heteroatom bond, and azo domains. The linker may comprise a domain derived from a click chemistry reaction (e.g., triazole, diazole, diazine, sulfide bond, maleimide ring, succinimide ring, ester, amide).

In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “mutation,” as used herein, refers to a substitution of a residue within a sequence, e.g., a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively the mutation could lead to overexpression of one or more genes involved in control of the cell cycle, thus leading to uncontrolled cell division and hence to cancer. Because of their nature, gain-of-function mutations are usually dominant.

The terms “non-naturally occurring” or “engineered” are used interchangeably and indicate the involvement of the hand of man. The terms, when referring to nucleic acid molecules or polypeptides (e.g., Cas9 or guanine oxidases) mean that the nucleic acid molecule or the polypeptide is at least substantially free from at least one other component with which they are naturally associated in nature and/or as found in nature (e.g., an amino acid sequence not found in nature).

The term “nucleic acid,” as used herein, refers to RNA as well as single- and/or double-stranded DNA. Nucleic acids may be naturally occurring, for example, in the context of a genome, a transcript, an mRNA, tRNA, rRNA, siRNA, snRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nucleic acid programmable D/RNA binding protein (napR/DNAbp)” refers to any protein that may associate (e.g., form a complex) with one or more nucleic acid molecules (i.e., which may broadly be referred to as a “napR/DNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napR/DNAbp embraces napDNAbps such as CRISPR Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system, also known as Cas13a), C2c3 (a type V CRISPR-Cas system, also known as Cas12c), dCas9, GeoCas9, CjCas9, Cas12a, Cas12b, Cas12g, Cas12h, Cas12i, Cas13b, Cas13c, Cas13d, Cas14, Csn2, Argonaute, nCas9, and circularly permuted Cas9 such as CP1012, CP1028, CP1041, CP1249, and CP1300. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

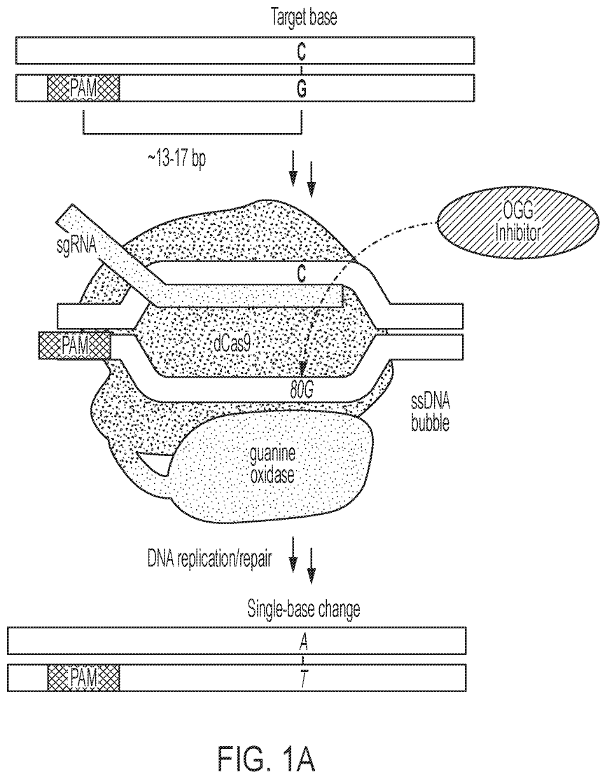

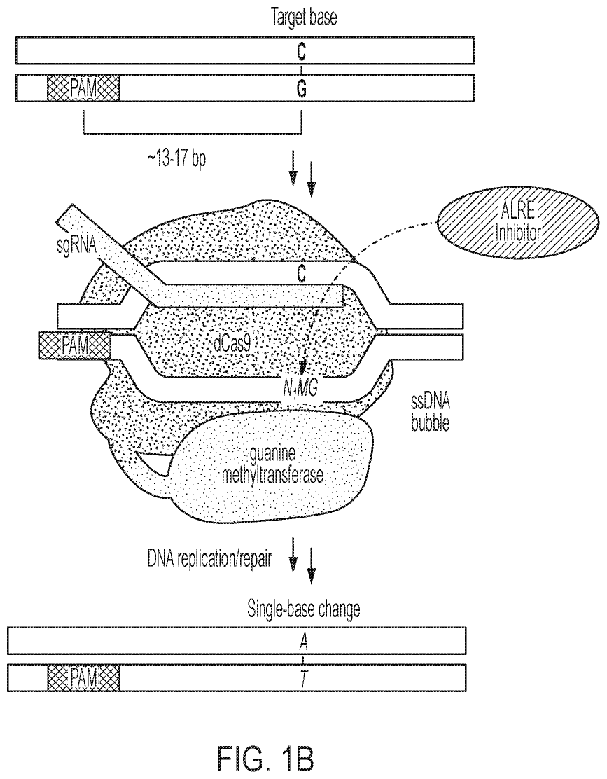

In some embodiments, the napR/DNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 and entitled “Delivery System For Functional Nucleases,” the entire contents of each are incorporated herein by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J. et al., Proc. Natl. Acad. Sci. U.S.A. 98:4658-4663(2001); “CRISPR RNA maturation by trans-encoded small RNA and host factor RNase III.” Deltcheva E. et al., Nature 471:602-607 (2011); and Jinek M. et al., “A programmable dual-RNA-guided DNA endonuclease in adaptive bacterial immunity.” Science 337:816-821 (2012), each of which is incorporated herein by reference.

The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

The term “napR/DNAbp-programming nucleic acid molecule” or equivalently “guide sequence” refers the one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system.

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences can be of any size and composition, for example more than 25, 25, 15, 12, 10, 8, 7, 6, 5, or 4 amino acids, but will preferably comprise at least a four to eight amino acid sequence known to function as a nuclear localization signal (NLS).

The term, as used herein, “nucleobase modification domain” or “modification domain” embraces any protein, enzyme, or polypeptide (or variant thereof) which is capable of modifying or replacing or exchanging a DNA or RNA molecule (e.g., a DNA or RNA nucleobase). Nucleobase modification domains may be naturally occurring, or may be engineered. For example, a nucleobase modification domain can include one or more DNA repair enzymes, for example, and an enzyme or protein involved in base excision repair (BER), nucleotide excision repair (NER), homology-dependent recombinational repair (HR), non-homologous end-joining repair (NHEJ), microhomology end-joining repair (MMEJ), mismatch repair (MMR), direct reversal repair, or other known DNA repair pathway. A nucleobase modification domain can have one or more types of enzymatic activities, including, but not limited to, endonuclease activity, polymerase activity, ligase activity, replication activity, and proofreading activity. Nucleobase modification domains include DNA or RNA-modifying enzymes and/or DNA or RNA-displacing enzymes, such as DNA methylases and oxidating enzymes (i.e., guanine methyltransferases and guanine oxidases), which covalently modify nucleobases leading in some cases to mutagenic corrections by way of normal cellular DNA repair and replication processes. Exemplary nucleobase modification domains include, but are not limited to, a guanine oxidase, a nuclease, a nickase, a recombinase, a methyltransferase, a methylase, an acetylase, an acetyltransferase, a transcriptional activator, or a transcriptional repressor domain. In some embodiments the nucleobase modification domain is a guanine oxidase (e.g., a guanine oxidase, such as an ScXDH).

As used herein, the terms “oligonucleotide” and “polynucleotide” can be used interchangeably to refer to a polymer of nucleotides (e.g., a string of at least three nucleotides).

The term “phage-assisted continuous evolution (PACE),” as used herein, refers to continuous evolution that employs phage as viral vectors. The general concept of PACE technology has been described, for example, in International PCT Application No. PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; and International PCT Application, PCT/US2016/027795, filed Apr. 15, 2016, published as WO 2016/168631 on Oct. 20, 2016, the entire contents of each of which are incorporated herein by reference.

The term “phage-assisted non-continuous evolution (PANCE),” as used herein, refers to non-continuous evolution that employs phage as viral vectors. The general concept of PANCE technology has been described, for example, in Suzuki T. et al., Crystal structures reveal an elusive functional domain of pyrrolysyl-tRNA synthetase, Nat Chem Biol. 13(12): 1261-1266 (2017), incorporated herein by reference in its entirety. Briefly, PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Following phage growth, an aliquot of infected cells is used to transfect a subsequent flask containing host E. coli. This process is continued until the desired phenotype is evolved, for as many transfers as required. Serial flask transfers have long served as a widely-accessible approach for laboratory evolution of microbes, and, more recently, analogous approaches have been developed for bacteriophage evolution. The PANCE system features lower stringency than the PACE system.

The term “promoter” is art-recognized and refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the specification provides vectors with appropriate promoters for driving expression of the nucleic acid sequences encoding the base editor fusion proteins (or one more individual components thereof).

The term “phage,” as used herein interchangeably with the term “bacteriophage,” refers to a virus that infects bacterial cells. Typically, phages consist of an outer protein capsid enclosing genetic material. The genetic material may be ssRNA, dsRNA, ssDNA, or dsDNA, in either linear or circular form. Phages and phage vectors are well known to those of skill in the art and non-limiting examples of phages that are useful for carrying out the methods provided herein are λ, T2, T4, T7, T12, R17, M13, MS2, G4, P1, P2, P4, Phi X174, N4, Φ6, and Φ29. In certain embodiments, the phage utilized in the present invention is M13. Additional suitable phages and host cells will be apparent to those of skill in the art and the invention is not limited in this aspect. For an exemplary description of additional suitable phages and host cells, see Elizabeth Kutter and Alexander Sulakvelidze: Bacteriophages: Biology and Applications. CRC Press; 1st edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1st edition (December, 2008), ISBN: 1588296822; Martha R. J. Clokie and Andrew M. Kropinski: Bacteriophages: Methods and Protocols, Volume 2: Molecular and Applied Embodiments (Methods in Molecular Biology) Humana Press; 1st edition (December 2008), ISBN: 1603275649; all of which are incorporated herein in their entirety by reference for disclosure of suitable phages and host cells as well as methods and protocols for isolation, culture, and manipulation of such phages).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, engineered, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a recombinase. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is an experimental organism. In some embodiments, the subject is a plant. In some embodiments, the subject is genetically engineered, e.g., a genetically engineered non-human subject. The subject may be of either sex and at any stage of development.

The term “target site” refers to a sequence within a nucleic acid molecule that is edited by a base editor (e.g., a dCas9-guanine oxidase fusion protein provided herein). The target site further refers to the sequence within a nucleic acid molecule to which a complex of the base editor and gRNA binds.

The term “vector,” as used herein, may refer to a nucleic acid that has been modified to encode a gene of interest, and that is able to enter into a host cell, mutate, and replicate within the host cell, and then transfer a replicated form of the vector into another host cell. Alternatively, the term “vector,” as used herein, may refer to a nucleic acid that has been modified to encode the base editor. Exemplary suitable vectors include viral vectors, such as retroviral vectors or bacteriophages and filamentous phage, and conjugative plasmids.

The term “viral particle,” as used herein, refers to a viral genome, for example, a DNA or RNA genome, that is associated with a coat of a viral protein or proteins, and, in some cases, with an envelope of lipids. For example, a phage particle comprises a phage genome packaged into a protein encoded by the wild type phage genome.

The term “viral vector,” as used herein, refers to a nucleic acid comprising a viral genome that, when introduced into a suitable host cell, can be replicated and packaged into viral particles able to transfer the viral genome into another host cell. The term “viral vector” extends to vectors comprising truncated or partial viral genomes. For example, in some embodiments, a viral vector is provided that lacks a gene encoding a protein essential for the generation of infectious viral particles. In suitable host cells, for example, host cells comprising the missing gene under the control of a conditional promoter, however, such truncated viral vectors can replicate and generate viral particles able to transfer the truncated viral genome into another host cell. In some embodiments, the viral vector is an adeno-associated virus (AAV) vector.

The terms “treatment,” “treat,” and “treating,” refer to a clinical intervention aimed to reverse, alleviate, delay the onset of, or inhibit the progress of a disease, disorder, or condition, or one or more symptoms thereof, as described herein. As used herein, the terms “treatment,” “treat,” and “treating” refer to a clinical intervention aimed to reverse, alleviate, delay the onset of, or inhibit the progress of a disease, disorder, or condition, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their prevention or recurrence.

As used herein, the term “variant” refers to a protein having characteristics that deviate from what occurs in nature, e.g., a “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant nucleobase modification domain is a nucleobase modification domain comprising one or more changes in amino acid residues of a guanine oxidase or guanine methyltransferase, as compared to the wild type amino acid sequences thereof. These changes include chemical modifications, including substitutions of different amino acid residues, as well as truncations. This term embraces functional fragments of the wild type amino acid sequence.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g., Cas9 protein, fusion protein, and fusion protein protein). Further polypeptides encompassed by the invention are polypeptides encoded by polynucleotides which hybridize to the complement of a nucleic acid molecule encoding a protein such as a Cas9 protein under stringent hybridization conditions (e.g., hybridization to filter bound DNA in 6× Sodium chloride/Sodium citrate (SSC) at about 45 degrees Celsius, followed by one or more washes in 0.2.times.SSC, 0.1% SDS at about 50-65 degrees Celsius), under highly stringent conditions (e.g., hybridization to filter bound DNA in 6× sodium chloride/Sodium citrate (SSC) at about 45 degrees Celsius, followed by one or more washes in 0.1×SSC, 0.2% SDS at about 68 degrees Celsius), or under other stringent hybridization conditions which are known to those of skill in the art (see, for example, Ausubel, F. M. et al., eds., 1989 Current Protocol in Molecular Biology, Green publishing associates, Inc., and John Wiley & Sons Inc., New York, at pp. 6.3.1-6.3.6 and 2.10.3).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a Cas9 protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

As used herein the term “wild type” is a term of the art understood by skilled persons and means the typical form of an organism, strain, gene or characteristic as it occurs in nature as distinguished from mutant or variant forms.

### DETAILED DESCRIPTION OF CERTAIN EMBODIMENTS

The present disclosure provides for guanine-to-thymine or “GTBE” (or cytosine-to-adenine or “CABE”) transversion base editors which comprise a napDNAbp, or more specifically, a napDNAbp (e.g., a dCas9 domain), fused to a nucleobase modification domain comprising a guanine oxidase or a guanine methyltransferase. The disclosed GTBE base editors are capable of converting a G:C nucleobase pair to an T:A nucleobase pair in a target nucleotide sequence of interest, e.g., a genome of a cell. The disclosed base editors may catalyze the conversion of a target guanine to a thymine via an oxidation reaction or an alkylation reaction of the guanine nucleobase.

The disclosed base editors also comprise GTBE base editors that catalyze the conversion of a target guanine to a thymine, and whereby the base-paired cytosine of the non-edited strand is subsequently converted to an adenine by the cell's replication and mismatch repair machinery.

In the methods of the present disclosure for which the oxidation approach is utilized, a targeted G in a nucleic acid of interest is first enzymatically oxidized to an 8-oxo-G. Steric rotation of the 8-oxo-G around the glycosidic bond is induced, presenting the Hoogsteen edge for base pairing. During replication or repair of the unmutated strand (which may be induced by a dead Cas9 in some embodiments), the 8-oxo-G is paired with a cytosine by a DNA polymerase. Without wishing to be bound by any particular theory, the cell recognizes the mismatch between 8-oxo-G and the cytosine on the unmutated strand and converts the cytosine to an adenine. Upon a subsequent round of replication or mismatch repair, the 8-oxo-G is converted to a thymine. A desired G-to-T transversion is thus achieved. Guanine oxidation is achieved by the targeted use of a fusion protein comprising a napDNAbp (e.g., dCas9 or nCas9) domain, a guanine oxidase domain, and optionally linkers interconnecting these domains (see FIG. 1A).

In the methods of the present disclosure for which the alkylation approach is utilized, a targeted G in a nucleic acid of interest is first enzymatically alkylated to a N₂,N₂-dimethyl-guanine or N₁-methyl-guanine. Alkylation will proceed to the N₂,N₂-dimethyl-guanine intermediate or the N₁-methyl-guanine intermediate based on which nitrogen center (N₁ or N₂) is more sterically or thermodynamically accessible to the enzyme. Steric rotation of the methylated guanine around the glycosidic bond may be induced, presenting the Hoogsteen edge for base pairing. During replication or repair of the unmutated strand (which may be induced by a dead Cas9 in some embodiments), the methylated guanine is paired with a cytosine by a DNA polymerase. Without wishing to be bound by any particular theory, the cell recognizes the mismatch between the methylated guanine and the cytosine on the unmutated strand and converts the cytosine to an adenine. Upon a subsequent round of replication or mismatch repair, the methylated guanine is converted to a thymine. A desired G-to-T transversion is thus achieved. Guanine methylation is achieved by the targeted use of a fusion protein comprising a napDNAbp (e.g., dCas9 or nCas9) domain, a guanine methyltransferase domain, and optionally linkers interconnecting these domains (see FIG. 1B).

In addition, the disclosure provides compositions comprising the GTBE base editors as described herein, e.g., fusion proteins comprising a dCas9 domain and a guanine oxidase domain, and one or more guide RNAs, e.g., a single-guide RNA (“sgRNA”). In addition, the instant specification provides for nucleic acid molecules encoding and/or expressing the GTBE base editors as described herein, as well as expression vectors and constructs for expressing the GTBE base editors described herein and/or a gRNA, host cells comprising said nucleic acid molecules and expression vectors and optionally vectors encoding one or more gRNAs, host cells comprising said GTBE base editors and optionally one or more gRNAs, and methods for delivering and/or administering nucleic acid-based embodiments described herein.

In some aspects, the present disclosure provides for methods of creating the transversion base editors, as well as methods of using the transversion base editors or nucleic acid molecules encoding the transversion base editors in applications including editing a nucleic acid molecule, e.g., a genome. In certain embodiments, methods of engineering the GTBE base editors provided herein is involve a phage-assisted continuous evolution (PACE) system or non-continuous system (e.g., PANCE), which may be utilized to evolve one or more components of a base editor (e.g., a guanine oxidase domain or guanine methyltransferase domain). In certain embodiments, following the successful evolution of one or more components of the GTBE base editor, methods of making the base editors comprise recombinant protein expression methodologies and techniques known to those of skill in the art.

The specification also provides methods for e editing a target nucleic acid molecule, e.g., a single nucleotide within a genome, with a base editing system described herein (e.g., in the form of an evolved base editor as described herein, or a vector or construct encoding a base editor). Such methods involve transducing (e.g., via transfection) cells with a plurality of complexes each comprising a base editor (e.g., a fusion protein comprising a dead Cas9 (dCas9) domain and a guanine oxidase domain) and optionally a gRNA molecule. In some embodiments, the gRNA is bound to the napDNAbp domain (e.g., dCas9 domain) of the fusion protein. In certain embodiments, the methods involve the transfection of nucleic acid constructs (e.g., plasmids) that each (or together) encode the components of a complex of a base editor and/or gRNA.

In certain embodiments, the disclosed methods comprise contacting a double-stranded DNA sequence with a complex comprising a fusion protein disclosed herein and a guide RNA, wherein the double-stranded DNA comprises a target G:C nucleobase pair; thereby substituting the guanine (G) of the G:C pair with a thymine. The disclosed methods may alternatively result in substitution of the guanine (G) of the G:C pair with a guanine derivative; such that the cell thereby subsequently substitutes the guanine derivative with a thymine during a subsequent round of replication. Exemplary guanine derivatives include 8-oxo-guanine, N₂,N₂-dimethyl-guanine, and N₁-methyl-guanine.

In certain embodiments of the disclosed methods, a nucleic acid construct (e.g., a plasmid) that encodes the fusion protein is transfected into the cell separately from the nucleic acid construct that encodes the gRNA molecule. In certain embodiments, these components are encoded on a single construct and transfected together.

In other embodiments, the methods disclosed herein involve the introduction into cells of a complex comprising a fusion protein and gRNA molecule that has been expressed and cloned outside of these cells.

It should be appreciated that any fusion protein, e.g., any of the fusion proteins described herein, may be introduced into the cell in any suitable way, either stably or transiently. In some embodiments, a fusion protein may be transfected into the cell. In some embodiments, the cell may be transduced or transfected with a nucleic acid construct that encodes a fusion protein. For example, a cell may be transduced (e.g., with a virus encoding a fusion protein) with a nucleic acid that encodes a fusion protein, or the translated fusion protein. As an additional example, a cell may be transfected (e.g., with a plasmid encoding a fusion protein) with a nucleic acid that encodes a fusion protein, or the translated fusion protein. Such transductions or transfections may be stable or transient. In some embodiments, cells expressing a fusion protein or containing a fusion protein may be transduced or transfected with one or more gRNA molecules, for example when the fusion protein comprises a Cas9 (e.g., dCas9) domain. In some embodiments, a plasmid expressing a fusion protein may be introduced into cells through electroporation, transient (e.g., lipofection), stable genome integration (e.g., piggybac), viral transduction, or other methods known to those of skill in the art.

In certain embodiments, the methods described herein further comprise (iii) cutting (or nicking) one strand of the double-stranded DNA, for example, the strand that includes the cytosine (C) of the target G:C nucleobase pair opposite the strand containing the target guanine (G) that is being mutated. This nicking step serves to direct mismatch repair machinery to the non-edited strand, ensuring that the modified nucleotide is not interpreted as a lesion by the cell's machinery. This nick may be created by the use of an nCas9.

The target nucleotide sequence may comprise a target sequence (e.g., a point mutation) associated with a disease, disorder, or condition, such as Marfan syndrome or Usher syndrome type 2a. The target sequence may comprise a T to G point mutation associated with a disease, disorder or condition, and wherein the oxidation of the mutant G base results in mismatch repair-mediated correction to a sequence that is not associated with a disease, disorder or condition. Alternatively, the target sequence may comprise an A to C point mutation associated with a disease, disorder, or condition, and wherein the GTBE-mediated conversion of the mutant C base that is paired with the mutant G base results in mismatch repair-mediated correction to a sequence that is not associated with a disease, disorder, or condition.

The target sequence can encode a protein, and where the point mutation is in a codon and results in a change in the amino acid encoded by the mutant codon as compared to a wild-type codon. The target sequence may also be at a splice site, and the point mutation results in a change in the splicing of an mRNA transcript as compared to the wild-type transcript. In addition, the target may be at a non-coding sequence of a gene, such as a gene promoter or gene repressor, and the point mutation results in increased or decreased expression of the gene.

Exemplary target genes include FBN1, in which a T to G point mutation at residue 136 affects connective tissue; and USHA2, in which a T to G point mutation at residue 934 results in hearing and/or vision loss. Additional target genes include human KRAS, HRAS and NRAS, for which an oncogenic phenotype is frequently caused by T:A to G:C point mutations. For some of these target genes, T:A to G:C point mutations introduce premature stop codons (UAA, UAG, UGA), resulting in nonsense mutations in protein coding regions. For all of the genetic disorders associated with the point mutations in these target genes, morbidity is high, and current treatment is not curative. Exemplary GTBEs disclosed herein correct these disease alleles in somatic cells, reducing or removing morbidity. In other embodiments, exemplary GTBEs disclosed herein may install disease-suppressing alleles in somatic cells.

Thus, in some aspects, the conversion of a mutant G results in correction of the nonsense mutation and restoration of the wild-type codon, which may result in the expression of a full-length, wild-type peptide sequence. For instance, the application of the base editors to target genetic sequences may induce a change in the mRNA transcript, such as restoring the mRNA transcript to a wild-type state.

The methods described herein may involve contacting a base editor with a target nucleotide sequence in vitro, ex vivo, or in vivo. In certain embodiments, this step of contacting occurs in a subject. In certain embodiments, the subject has been diagnosed with a disease, disorder, or condition, such as, but not limited to, a disease, disorder, or condition associated with a point mutation in the FBN1 gene or the USHA2 gene.

In another aspect, the specification discloses a pharmaceutical composition comprising any one of the presently disclosed base editors (or fusion proteins). In one aspect, the specification discloses a pharmaceutical composition comprising any one of the presently disclosed complexes of fusion proteins and gRNA. In one aspect, the specification discloses a pharmaceutical composition comprising polynucleotides encoding the fusion proteins disclosed herein and polynucleotides encoding a gRNA, or polynucleotides encoding both. In another aspect, the specification discloses a pharmaceutical composition comprising any one of the presently disclosed vectors.

### I. G-to-T Transversion Base-Editors

The present disclosure provides G-to-T (or C-to-A) transversion base editors comprising (i) a napDNAbp domain and (ii) a nucleobase modification domain that is capable of facilitating the conversion of a G to a T in a target nucleotide sequence, e.g., a genome. The nucleobase modification domain may be a guanine oxidase that enzymatically oxidizes a guanine nucleobase of a G:C nucleobase pair. In other embodiments, the nucleobase modification domain is a guanine methyltransferase that enzymatically alkylates the guanine nucleobase. In both of these embodiments, the G:C nucleobase pair is ultimately converted to a T:A nucleobase pair.

The various domains of the GTBE base editors (or fusion proteins) described herein may be obtained as a result of mutagenizing a reference base editor (or a component or domain thereof) by a directed evolution process, e.g., a continuous evolution method (e.g., PACE) or a non-continuous evolution method (e.g., PANCE or other discrete plate-based selections). In various embodiments, the disclosure provides a base editor that has one or more amino acid variations introduced into its amino acid sequence relative to the amino acid sequence of the reference base editor. The base editor may include variants in one or more components or domains of the base editor (e.g., variants introduced into a Cas9 domain, variants introduced into the nucleobase modification domain, or a variant introduced into both of these domains).

The nucleobase modification domain may be engineered in any way known to those of skill in the art. For example, the nucleobase modification domain may be evolved from a reference protein that is an RNA modifying enzyme (e.g., a guanine oxidase may be evolved from a xanthine dehydrogenase) and evolved using PACE, PANCE, or other plate-based evolution methods to obtain a DNA modifying version of the nucleobase modification domain, which can then be used in the fusion proteins described herein. For example, the disclosed guanine oxidase and/or guanine methyltransferase variants may be at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the reference enzyme. In some embodiments, the guanine oxidase and/or guanine methyltransferase variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference enzyme. In other embodiments, the guanine oxidase and/or guanine methyltransferase variant comprises multiple amino acid stretches having about 99.9% identity, followed by one or more stretches having at least about 90% or at least about 95% identity, followed by stretches of having about 99.9% identity, to the corresponding amino acid sequence of the reference enzyme.

(A) Cas9 Domains

The GTBE base editors provided by the instant specification include any suitable napDNAbp domains. Exemplary napDNAbp domains comprise a Cas9 domain or variant thereof, including naturally-occurring or engineered variant of Cas9. The base editors described herein may comprise fusion proteins in which the Cas9 domain has not been evolved, but wherein one or more other base editor domains (e.g., a guanine oxidase domain) have been evolved.

The napDNAbp domain may comprise any CRISPR associated protein, including, but not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, and homologs and modified versions thereof. These enzymes are known; for example, the amino acid sequence of S. pyogenes Cas9 protein may be found in the SwissProt database under accession number Q99ZW2. In some embodiments, the napDNAbp has DNA cleavage activity, such as Cas9.

In some embodiments, the napDNAbp is a single effector of a microbial CRISPR-Cas system. Single effectors of microbial CRISPR-Cas systems include, without limitation, Cas9, Cpf1, C2c1, C2c2, C2c3, CjCas9, Cas12a, Cas12b, Cas12c, Cas12d, Cas12g, Cas12h, Cas12i, Cas13b, Cas13c, Cas13d, Cas14, Csn2, and GeoCas9. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell Biol., 2015 Nov. 5; 60(3): 385-397, which is incorporated herein by reference. Effectors of two of the systems, C2c1 and C2c3, contain RuvC-like endonuclease domains related to Cpf1. A third system, C2c2, contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, incorporated herein by reference. In vitro biochemical analysis of C2c2 in Leptotrichia shahii has shown that C2c2 is guided by a single CRISPR RNA and can be programmed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), incorporated herein by reference.

In various embodiments, the napDNAbp domain is derived from Staphylococcus pyogenes Cas9 (SpCas9) or derived from Staphylococcus aureus (SaCas9), both of which have been widely used as a tool for genome engineering. In some embodiments, the napDNAbp domain is a Cas9 is from S. pneumoniae. These Cas9 proteins are large, multi-domain proteins containing two distinct nuclease domains. Point mutations can be introduced into Cas9 to abolish completely or partially its nuclease activity, resulting in a dead Cas9 (dCas9) or nickase Cas9 (nCas9) that still retains its ability to bind a nucleic acid in a sgRNA-programmed manner. In principle, when fused to a modification domain, the Cas9 domain can target the modification domain to virtually any DNA sequence simply by binding an appropriate sgRNA.

In other embodiments, the napDNAbp domain is a Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP 820832.1); Listeria innocua (NCBI Ref: NP 472073.1); Campylobacter jejuni (NCBI Ref: YP 002344900.1); or Neisseria. meningitidis (NCBI Ref: YP 002342100.1).

In some embodiments, the Cas9 directs cleavage of one or both strands at the location of a target sequence, such as within the target sequence and/or within the complement of the target sequence. In some embodiments, the Cas9 directs cleavage of one or both strands within about 1, 2, 3, 4, 5, 6, 7, 8, 9, 10, 15, 20, or more base pairs from the 3′ terminus or the 5′ terminus of a target sequence.

In some embodiments, the napDNAbp is mutated with respect to a corresponding wild-type enzyme such that the mutated napDNAbp lacks the ability to cleave one or both strands of a target polynucleotide containing a target sequence. In particular embodiments, an aspartate-to-alanine substitution (D10A) in the RuvC1 catalytic domain of S. pyogenes Cas9 converts Cas9 from a nuclease that cleaves both strands to a nickase that nicks the targeted strand, or the strand that is complementary to the gRNA. A histidine-to-alanine substitution (H840A) in the HNH catalytic domain of S. pyogenes Cas9 generates a nick on the strand that is displaced by the gRNA during strand invasion, also referred to herein as the non-edited strand. The single catalytically active nuclease site of the nCas9 leaves a nick in the non-edited strand, which will direct mismatch repair machinery to read (rather than remove) the modified base during repair (i.e., a substituted guanine or guanine derivative at the target site). Other examples of mutations that render Cas9 a nickase include, without limitation, N854A and N863A in SpCas9, and corresponding mutations in other wild-type Cas9 proteins or variants thereof. Reference is made to U.S. Pat. No. 8,945,839, incorporated herein by reference.

In some embodiments, the napDNAbp domains disclose herein are referred to as “Cas9 variants.” A Cas9 variant shares homology to Cas9. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type Cas9.

In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length. In some embodiments, the fragment binds crRNA and tracrRNA or sgRNA, but does not comprise a functional nuclease domain, e.g., in that it comprises only a truncated version of a nuclease domain or no nuclease domain at all.

In some embodiments, wild type Cas9 corresponds to Cas9 from Streptococcus pyogenes MGAS1882 (NCBI Reference Sequence: NC_017053.1). In other embodiments, wild type Cas9 corresponds to Cas9 from S. pyogenes M1 GAS (NCBI Reference Sequence: NC_002737.2). In some embodiments, variants or homologues of dCas9 (e.g., variants of Cas9 from S. pyogenes (NCBI Reference Sequence: NC_017053.1)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1) are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

It should be appreciated that additional Cas9 proteins (e.g., a nuclease dead Cas9 (dCas9), a Cas9 nickase (nCas9), or a nuclease active Cas9), including variants and homologs thereof, are within the scope of this disclosure. Exemplary Cas9 proteins include, without limitation, those provided below. In some embodiments, the Cas9 protein is a nuclease dead Cas9 (dCas9). In some embodiments, the dCas9 is derived from S. pyogenes and comprises the amino acid sequence set forth as SEQ ID NO: 32. In other embodiments, the Cas9 protein is a Cas9 nickase (nCas9). In some embodiments, the nCas9 is derived from S. pyogenes and comprises the amino acid sequence set forth as SEQ ID NO: 9.

In certain embodiments, the base editors of the invention can include a catalytically inactive Cas9 (dCas9) that comprises an amino acid sequence that is at least 80%, 85%, 90%, 95%, 98%, or 99% identical to the amino acid sequence of:

(SEQ ID NO: 32)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD, or a variant thereof.

In other embodiments, the base editors may comprise a Cas9 nickase (nCas9) that comprises an amino acid sequence that is at least 80%, 85%, 90%, 95%, 98%, or 99% identical to the amino acid sequence of (D10A mutation is bolded and underlined):

(SEQ ID NO: 9)
DKKYSIGL [symbol] IGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD, and may be a variant thereof.

In still other embodiments, the base editors may comprise a catalytically active Cas9 that comprises an amino acid sequence that is at least 80%, 85%, 90%, 95%, 98%, or 99% identical to the amino acid sequence of:

(SEQ ID NO: 33)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD (wild-type SpCas9).

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises the amino acid sequence SEQ ID NO: 45. In some embodiments, the SaCas9 comprises a N579X mutation of SEQ ID NO: 45, wherein X is any amino acid except for N. In some embodiments, the SaCas9 comprises a N579A mutation of SEQ ID NO: 45. In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT PAM sequence.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to the amino acid set forth as SEQ ID NO: 45, below. In some embodiments, the Cas9 domain of any of the fusion proteins provided herein comprises the amino acid sequence of SEQ ID NO: 45. In some embodiments, the Cas9 domain of any of the fusion proteins provided herein consists of the amino acid sequence of SEQ ID NO: 45.

An exemplary SaCas9 amino acid sequence is:

(SEQ ID NO: 45)
KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG

An additional napDNAbp domain with altered PAM specificity, such as a domain having at least 80%, at least 85%, at least 90%, at least 95%, or at least 99% sequence identity with wild type Geobacillus thermodenitrificans Cas9 (SEQ ID NO: 49, GeoCas9) may be used.

(SEQ ID NO: 100)
MKYKIGLDIGITSIGWAVINLDIPRIEDLGVRIFDRAENPKTGESLALPR
RLARSARRRLRRRKHRLERIRRLFVREGILTKEELNKLFEKKHEIDVWQL
RVEALDRKLNNDELARILLHLAKRRGFRSNRKSERTNKENSTMLKHIEEN
QSILSSYRTVAEMVVKDPKFSLHKRNKEDNYTNTVARDDLEREIKLIFAK
QREYGNIVCTEAFEHEYISIWASQRPFASKDDIEKKVGFCTFEPKEKRAP
KATYTFQSFTVWEHINKLRLVSPGGIRALTDDERRLIYKQAFHKNKITFH
DVRTLLNLPDDTRFKGLLYDRNTTLKENEKVRFLELGAYHKIRKAIDSVY
GKGAAKSFRPIDFDTFGYALTMFKDDTDIRSYLRNEYEQNGKRMENLADK
VYDEELIEELLNLSFSKFGHLSLKALRNILPYMEQGEVYSTACERAGYTF
TGPKKKQKTVLLPNIPPIANPVVMRALTQARKVVNAIIKKYGSPVSIHIE
LARELSQSFDERRKMQKEQEGNRKKNETAIRQLVEYGLTLNPTGLDIVKF
KLWSEQNGKCAYSLQPIEIERLLEPGYTEVDHVIPYSRSLDDSYTNKVLV
LTKENREKGNRTPAEYLGLGSERWQQFETFVLTNKQFSKKKRDRLLRLHY
DENEENEFKNRNLNDTRYISRFLANFIREHLKFADSDDKQKVYTVNGRIT
AHLRSRWNFNKNREESNLHHAVDAAIVACTTPSDIARVTAFYQRREQNKE
LSKKTDPQFPQPWPHFADELQARLSKNPKESIKALNLGNYDNEKLESLQP
VFVSRMPKRSITGAAHQETLRRYIGIDERSGKIQTVVKKKLSEIQLDKTG
HFPMYGKESDPRTYEAIRQRLLEHNNDPKKAFQEPLYKPKKNGELGPIIR
TIKIIDTTNQVIPLNDGKTVAYNSNIVRVDVFEKDGKYYCVPIYTIDMMK
GILPNKAIEPNKPYSEWKEMTEDYTFRFSLYPNDLIRIEFPREKTIKTAV
GEEIKIKDLFAYYQTIDSSNGGLSLVSHDNNFSLRSIGSRTLKRFEKYQV
DVLGNIYKVRGEKRVGVASSSHSKAGETIRPL

In some embodiments, a napDNAbp domain refers to a Cas9 or Cas9 homolog from archaea (e.g., nanoarchaea), which constitute a domain and kingdom of single-celled prokaryotic microbes. In some embodiments, a napDNAbp domain may comprise a CasX (also referred to as Cas12e) or CasY (now referred to as Cas12d) omain, which have been described in, for example, Burstein et al., “New CRISPR-Cas systems from uncultivated microbes.” Cell Res. 2017 Feb. 21. doi: 10.1038/cr.2017.21, and Liu et al., “CasX enzymes comprise a distinct family of RNA-guided genome editors,” Nature. 2019; 566(7743):218-223, each of which is incorporated herein by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, napDNAbp domain refers to CasX, or a variant of CasX. In some embodiments, napDNAbp domain refers to a CasY, or a variant of CasY. It should be appreciated that other RNA-guided DNA binding proteins may be used as a napDNAbp and are within the scope of this disclosure. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein.

In other embodiments, the napDNAbp domain may comprise, without limitation, Cpf1, C2c1, C2c2 (Cas13a), C2c3 (Cas12c), GeoCas9, CjCas9, Cas12a, Cas12b, Cas12g, Cas12h, Cas12i, Cas13b, Cas13c, Cas13d, Cas14, Csn2, Argonaute, evolved Cas9 domains (xCas9) and circularly permuted Cas9 proteins such as CP1012, CP1028, CP1041, CP1249, and CP1300.

An example of a napDNAbp that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae have been shown to have efficient genome-editing activity in human cells. See Zetsche et al., Cell 2015; 163(3):759-771. Cpf1 proteins are known in the art and have been described previously, for example Yamano et al., “Crystal structure of Cpf1 in complex with guide RNA and target DNA.” Cell (165) 2016: 949-962, which is incorporated herein by reference.

Also useful in the presently disclosed base editors. compositions and methods are nuclease-inactive Cpf1 (dCpf1) variants that may be used as a guide nucleotide sequence-programmable DNA-binding protein domain. The Cpf1 protein has a RuvC-like endonuclease domain that is similar to the RuvC domain of Cas9 but does not have an HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 inactivates Cpf1 nuclease activity. In some embodiments, the dCpf1 useful in the present disclosure comprises mutations corresponding to D917A, E1006A, D1255A, D917A/E1006A, D917A/D1255A, E1006A/D1255A, or D917A/E1006A/D1255A of Francisella novicida Cpf1 in SEQ ID NO: 34. It is to be understood that any mutations, e.g., substitution mutations, deletions, or insertions that inactivate the RuvC domain of Cpf1, may be used in accordance with the present disclosure.

In some embodiments, the nucleic acid programmable DNA binding protein (napDNAbp) of any of the fusion proteins provided herein may be a Cpf1 protein. In some embodiments, the Cpf1 protein is a Cpf1 nickase (nCpf1). In some embodiments, the Cpf1 protein is a nuclease inactive Cpf1 (dCpf1).

For example, a napDNAbp domain with altered PAM specificity, such as a domain with at least 80%, at least 85%, at least 90%, at least 95%, or at least 99% sequence identity with wild type Francisella novicida Cpf1 (SEQ ID NO: 34) (the D917, E1006, and D1255 residues are bolded and underlined), may be used:

(SEQ ID NO: 34)
MSIYQEFVNKYSLSKTLRFELIPQGKTLENIKARGLILDDEKRAKDYKK
AKQIIDKYHQFFIEEILSSVCISEDLLQNYSDVYFKLKKSDDDNLQKDF
KSAKDTIKKQISEYIKDSEKFKNLFNQNLIDAKKGQESDLILWLKQSKD
NGIELFKANSDITDIDEALEIIKSFKGWTTYFKGFHENRKNVYSSNDIP
TSIIYRIVDDNLPKFLENKAKYESLKDKAPEAINYEQIKKDLAEELTFD
IDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLE
DDSDVVTTMQSFYEQIAAFKTVEEKSIKETLSLLFDDLKAQKLDLSKIY
FKNDKSLTDLSQQVFDDYSVIGTAVLEYITQQIAPKNLDNPSKKEQELI
AKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHK
LKIFHISQSEDKANILDKDEHFYLVFEECYFELANIVPLYNKIRNYITQ
KPYSDEKFKLNFENSTLANGWDKNKEPDNTAILFIKDDKYYLGVMNKKN
NKIFDDKAIKENKGEGYKKIVYKLLPGANKMLPKVFFSAKSIKFYNPSE
DILRIRNHSTHTKNGSPQKGYEKFEFNIEDCRKFIDFYKQSISKHPEWK
DFGFRFSDTQRYNSIDEFYREVENQGYKLTFENISESYIDSVVNQGKLY
LFQIYNKDFSAYSKGRPNLHTLYWKALFDERNLQDVVYKLNGEAELFYR
KQSIPKKITHPAKEAIANKNKDNPKKESVFEYDLIKDKRFTEDKFFFHC
PITINFKSSGANKFNDEINLLLKEKANDVHILSI [symbol] RGERHLAYYTLVDG
KGNIIKQDTFNIIGNDRMKTNYHDKLAAIEKDRDSARKDWKKINNIKEM
KEGYLSQVVHEIAKLVIEYNAIVVF [symbol] DLNFGFKRGRFKVEKQVYQKLEK
MLIEKLNYLVFKDNEFDKTGGVLRAYQLTAPFETFKKMGKQTGIIYYVP
AGFTSKICPVTGFVNQLYPKYESVSKSQEFFSKFDKICYNLDKGYFEFS
FDYKNFGDKAAKGKWTIASFGSRLINFRNSDKNHNWDTREVYPTKELEK
LLKDYSIEYGHGECIKAAICGESDKKFFAKLTSVLNTILQMRNSKTGTE
LDYLISPVADVNGNFFDSRQAPKNMPQDA [symbol] ANGAYHIGLKGLMLLGRIK
NNQEGKKLNLVIKNEEYFEFVQNRNN

In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to SEQ ID NO: 34 and comprises mutations corresponding to D917A, E1006A, D1255A, D917A/E1006A, D917A/D1255A, E1006A/D1255A, or D917A/E1006A/D1255A. It should be appreciated that Cpf1 from other bacterial species may also be used in accordance with the present disclosure.

In some embodiments, the nucleic acid programmable DNA binding protein (napDNAbp) is a nucleic acid programmable DNA binding protein that does not require a canonical (NGG) PAM sequence. In some embodiments, the napDNAbp is an Argonaute protein, e.g., an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA target site. In contrast to Cas9, the NgAgo-gDNA system does not require a protospacer-adjacent motif (PAM). Using a NgAgo (dNgAgo) can greatly expand the bases that may be targeted. The characterization and use of NgAgo have been described in Gao et al., Nat Biotechnol., 34(7): 768-73 (2016), PubMed PMID: 27136078; Swarts et al., Nature, 507(7491): 258-61 (2014); and Swarts et al., Nucleic Acids Res. 43(10) (2015): 5120-9, each of which is incorporated herein by reference. The sequence of Natronobacterium gregoryi Argonaute is provided in SEQ ID NO: 42.

The disclosed fusion proteins may comprise a napDNAbp domain having at least 80%, at least 85%, at least 90%, at least 95%, or at least 99% sequence identity with wild type Natronobacterium gregoryi Argonaute (SEQ ID NO: 42).

(SEQ ID NO: 42)
MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

In some embodiments, the napDNAbp is a prokaryotic homolog or variant of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. (2009), 4:29, doi: 10.1186/1745-6150-4-29, which is incorporated herein by reference. In some embodiments, the napDNAbp is a Marinitoga piezophila Argonaute (MpAgo) protein. The CRISPR-associated Marinitoga piezophila Argonaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, which are incorporated herein by reference). It should be appreciated that other Argonaute proteins may be used, and are within the scope of this disclosure.

The crystal structure of Alicyclobaccillus acidoterrastris C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell Biol., 2017 Jan. 19; 65(2):310-322, which are incorporated herein by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, which are incorporated herein by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins provided herein may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the napDNAbp is a naturally-occurring C2c1, C2c2, or C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 3 or 4. In some embodiments, the napDNAbp comprises an amino acid sequence of any one SEQ ID NOs: 3 or 4. It should be appreciated that C2c1, C2c2, or C2c3 from other bacterial species may also be used in accordance with the present disclosure.

In some embodiments, the nucleic acid programmable DNA binding protein (napDNAbp) of any of the fusion proteins provided herein may be a CjCas9, Cas12a, Cas12b, Cas12g, Cas12h, Cas12i, Cas13b, Cas13c, Cas13d, Cas14, Csn2, and GeoCas9. CjCas9 is described and characterized in Kim et al., Nat Commun. 2017; 8:14500 and Dugar et al., Molecular Cell 2018; 69:893-905, incorporated herein by reference. GeoCas9 is described and characterized in Harrington et al. Nat Commun. 2017; 8(1):1424, incorporated herein by reference. The Cas12a, Cas12b, Cas12g, Cas12h and Cas12i proteins are described and characterized in, e.g., Yan et al., Science, 2019; 363(6422): 88-91, Murugan et al. The Revolution Continues: Newly Discovered Systems Expand the CRISPR-Cas Toolkit, Molecular Cell 2017; 68(1):15-25, each of which are incorporated herein by reference. Cas14 is characterized and described in Harrington et al. Science 2018; 362(6416):839-842, incorporated herein by reference. Cas13b, Cas13c and Cas13d are described and characterized in Smargon et al., Molecular Cell 2017, Cox et al., Science 2017, and Yan et al. Molecular Cell 70, 327-339.e5 (2018), each of which are incorporated herein by reference. Csn2 is described and characterized in Koo Y., Jung D. K., and Bae E. PLoS One. 2012; 7:e33401, incorporated herein by reference.

C2c1 (uniprot.org/uniprot/T0D7A2#)

sp|T0D7A2|C2C1_ALIAG CRISPR-associated endonuclease C2c1 OS=Alicyclobacillus acidoterrestris (strain ATCC 49025/DSM 3922/CIP 106132/NCIMB 13137/GD3B) GN=c2c1 PE=1 SV=1

(SEQ ID NO: 3)
MAVKSIKVKLRLDDMPEIRAGLWKLHKEVNAGVRYYTEWLSLLRQENLYR
RSPNGDGEQECDKTAEECKAELLERLRARQVENGHRGPAGSDDELLQLAR
QLYELLVPQAIGAKGDAQQIARKFLSPLADKDAVGGLGIAKAGNKPRWVR
MREAGEPGWEEEKEKAETRKSADRTADVLRALADFGLKPLMRVYTDSEMS
SVEWKPLRKGQAVRTWDRDMFQQAIERMMSWESWNQRVGQEYAKLVEQKN
RFEQKNFVGQEHLVHLVNQLQQDMKEASPGLESKEQTAHYVTGRALRGSD
KVFEKWGKLAPDAPFDLYDAEIKNVQRRNTRRFGSHDLFAKLAEPEYQAL
WREDASFLTRYAVYNSILRKLNHAKMFATFTLPDATAHPIWTRFDKLGGN
LHQYTFLFNEFGERRHAIRFHKLLKVENGVAREVDDVTVPISMSEQLDNL
LPRDPNEPIALYFRDYGAEQHFTGEFGGAKIQCRRDQLAHMHRRRGARDV
YLNVSVRVQSQSEARGERRPPYAAVFRLVGDNHRAFVHFDKLSDYLAEHP
DDGKLGSEGLLSGLRVMSVDLGLRTSASISVFRVARKDELKPNSKGRVPF
FFPIKGNDNLVAVHERSQLLKLPGETESKDLRAIREERQRTLRQLRTQLA
YLRLLVRCGSEDVGRRERSWAKLIEQPVDAANHMTPDWREAFENELQKLK
SLHGICSDKEWMDAVYESVRRVWRHMGKQVRDWRKDVRSGERPKIRGYAK
DVVGGNSIEQIEYLERQYKFLKSWSFFGKVSGQVIRAEKGSRFAITLREH
IDHAKEDRLKKLADRIIMEALGYVYALDERGKGKWVAKYPPCQLILLEEL
SEYQFNNDRPPSENNQLMQWSHRGVFQELINQAQVHDLLVGTMYAAFSSR
FDARTGAPGIRCRRVPARCTQEHNPEPFPWWLNKFVVEHTLDACPLRADD
LIPTGEGEIFVSPFSAEEGDFHQIHADLNAAQNLQQRLWSDFDISQIRLR
CDWGEVDGELVLIPRLTGKRTADSYSNKVFYTNTGVTYYERERGKKRRKV
FAQEKLSEEEAELLVEADEAREKSVVLMRDPSGIINRGNWTRQKEFWSMV
NQRIEGYLVKQIRSRVPLQDSACENTGDI

C2c2 (uniprot.org/uniprot/P0DOC6)

>sp|P0DOC6|C2C2_LEPSD CRISPR-associated endoribonuclease C2c2 OS=Leptotrichia shahii (strain DSM 19757/CCUG 47503/CIP 107916/JCM 16776/LB37) GN=c2c2 PE=1 SV=1

(SEQ ID NO: 4)
MGNLFGHKRWYEVRDKKDFKIKRKVKVKRNYDGNKYILNINENNNKEKID
NNKFIRKYINYKKNDNILKEFTRKFHAGNILFKLKGKEGIIRIENNDDFL
ETEEVVLYIEAYGKSEKLKALGITKKKIIDEAIRQGITKDDKKIEIKRQE
NEEEIEIDIRDEYTNKTLNDCSIILRIIENDELETKKSIYEIFKNINMSL
YKIIEKIIENETEKVFENRYYEEHLREKLLKDDKIDVILTNFMEIREKIK
SNLEILGFVKFYLNVGGDKKKSKNKKMLVEKILNINVDLTVEDIADFVIK
ELEFWNITKRIEKVKKVNNEFLEKRRNRTYIKSYVLLDKHEKFKIERENK
KDKIVKFFVENIKNNSIKEKIEKILAEFKIDELIKKLEKELKKGNCDTEI
FGIFKKHYKVNFDSKKFSKKSDEEKELYKIIYRYLKGRIEKILVNEQKVR
LKKMEKIEIEKILNESILSEKILKRVKQYTLEHIMYLGKLRHNDIDMTTV
NTDDFSRLHAKEELDLELITFFASTNMELNKIFSRENINNDENIDFFGGD
REKNYVLDKKILNSKIKIIRDLDFIDNKNNITNNFIRKFTKIGTNERNRI
LHAISKERDLQGTQDDYNKVINIIQNLKISDEEVSKALNLDVVFKDKKNI
ITKINDIKISEENNNDIKYLPSFSKVLPEILNLYRNNPKNEPFDTIETEK
IVLNALIYVNKELYKKLILEDDLEENESKNIFLQELKKTLGNIDEIDENI
IENYYKNAQISASKGNNKAIKKYQKKVIECYIGYLRKNYEELFDFSDFKM
NIQEIKKQIKDINDNKTYERITVKTSDKTIVINDDFEYIISIFALLNSNA
VINKIRNRFFATSVWLNTSEYQNIIDILDEIMQLNTLRNECITENWNLNL
EEFIQKMKEIEKDFDDFKIQTKKEIFNNYYEDIKNNILTEFKDDINGCDV
LEKKLEKIVIFDDETKFEIDKKSNILQDEQRKLSNINKKDLKKKVDQYIK
DKDQEIKSKILCRIIFNSDFLKKYKKEIDNLIEDMESENENKFQEIYYPK
ERKNELYIYKKNLFLNIGNPNFDKIYGLISNDIKMADAKFLFNIDGKNIR
KNKISEIDAILKNLNDKLNGYSKEYKEKYIKKLKENDDFFAKNIQNKNYK
SFEKDYNRVSEYKKIRDLVEFNYLNKIESYLIDINWKLAIQMARFERDMH
YIVNGLRELGIIKLSGYNTGISRAYPKRNGSDGFYTTTAYYKFFDEESYK
KFEKICYGFGIDLSENSEINKPENESIRNYISHFYIVRNPFADYSIAEQI
DRVSNLLSYSTRYNNSTYASVFEVFKKDVNLDYDELKKKFKLIGNNDILE
RLMKPKKVSVLELESYNSDYIKNLIIELLTKIENTNDTL

The Cas9 domains of the fusion proteins provided herein may comprise a circularly permuted Cas9 domain such as CP1012, CP1028, CP1041, CP1249, and CP1300. In particular embodiments, the Cas9 domain may comprise CP1028. Circularly permuted Cas9 domains refer to any Cas9 protein or variant thereof that occurs as a circular permutant, whereby its N- and C-termini have been topically rearranged. Such circularly permuted Cas9 domains retain the ability to bind DNA when complexed with a guide RNA (gRNA) and may recognize non-NGG protospacer adjacent motifs. Circularly permuted Cas9 proteins are described in Huang et al., Nat Biotechnol. 2019; 37(6):626-631 and U.S. Provisional Application No. 62/884,459, filed Aug. 8, 2019, each of which is incorporated herein by reference.

Cas9 domains evolved by continuous and non-continuous evolution (xCas9) are described in International Patent Publication No. PCT/US2019/47996, filed Aug. 23, 2019, incorporated herein by reference.

(B) Guanine Oxidases

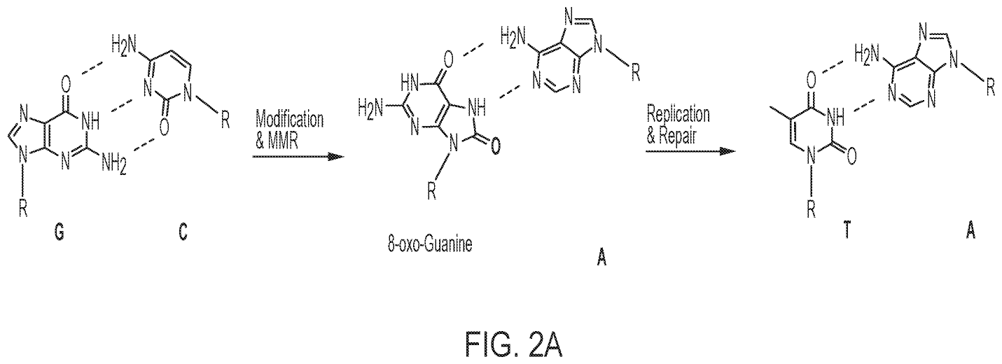

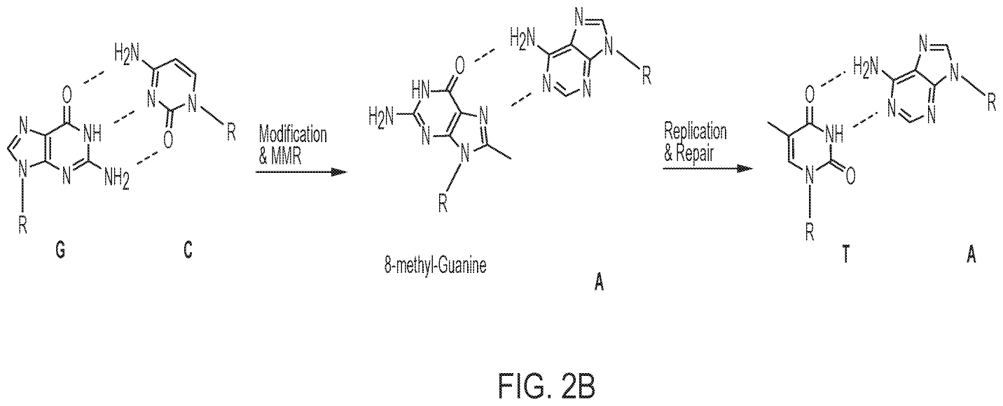

In various embodiments, the GTBE (and CABE) base editors provided herein comprise a guanine oxidase nucleobase modification domain (FIG. 1A). Any oxidase that is adapted to accept guanine nucleotide substrates are useful in the base editors and methods of editing disclosed herein. A guanine oxidase is an enzyme that catalyzes the oxidation of a guanine nucleobase to form 8-oxo-guanine (see FIG. 2A).

The guanine oxidase may comprise a naturally-occurring or modified oxidase, such as an oxidase engineered from a reference enzyme such as molybdenum-containing dioxygenase xanthine dehydrogenase, which accepts xanthine as a substrate. Modified oxidases may be obtained by, e.g., evolving a reference oxidase or dioxygenase (e.g., an RNA modification enzyme) evolved using a continuous evolution process (e.g., PACE) or non-continuous evolution process (e.g., PANCE or plate-based selections) described herein so that the oxidase/dioxygenase is effective on a nucleic acid target. See Falnes, P. Ø. & Rognes, T. DNA repair by bacterial AlkB proteins, Res. Microbiol. (2003) 154(8): 531-538; Ito, S. et al., Tet proteins can convert 5-methylcytosine to 5-formylcytosine and 5-carboxylcytosine, Science (2011) 333(6047): 1300-1303; Fortini, P. et al., 8-Oxoguanine DNA damage: at the crossroad of alternative repair pathways, Mutat. Res. (2003) 531(1-2): 127-39; Leonard, G. A. et al., Conformation of guanine-8-oxoadenine base pairs in the crystal structure of d(CGCGAATT(O8A)GCG) (SEQ ID NO: 30), Biochem. (1992) 31(36): 8415-8420; Ohe, T. & Watanabe, Y. Purification and Properties of Xanthine Dehydrogenase from Streptomyces cyanogenus, J. Biochem. 86:45-53, (1979 each of which is herein incorporated by reference.

In one embodiment, the guanine oxidase is a wild-type guanine oxidase, or a variant thereof, that oxidizes a guanine in DNA. In certain embodiments, the guanine oxidase is a xanthine dehydrogenase, or a variant thereof. In certain embodiments, the xanthine dehydrogenase is a Streptomyces cyanogenus xanthine dehydrogenase (ScXDH) or variant thereof. In other embodiments, the xanthine dehydrogenase or variant thereof is derived from C. capitata, N. crassa, M. hansupus, E. cloacae, S. snoursei, S. albulus, S. himastatinicus, or S. lividans.

In other embodiments, the guanine oxidase is a cytochrome P450 enzyme, or a variant thereof. In certain embodiments, the guanine oxidase is a human CYP1A2, CYP2A6 or CYP3A6, or a variant thereof.

In other embodiments, the guanine oxidase is a TET-oxidase, or a variant thereof. In certain embodiments, the guanine oxidase is a TET1, TET1-CD, TET2 or TET3, or a variant thereof.

In other embodiments, the guanine oxidase is an AlkB, or a variant thereof. In certain embodiments, the guanine oxidase is a bacterial AlkB, or a variant thereof. In other embodiments, the guanine oxidase is a human ABH3, or a variant thereof.

In various embodiments, the guanine oxidase comprises any one of the amino acid sequences of SEQ ID NOs: 5-8, SEQ ID NO: 10, SEQ ID NOs: 15-20, SEQ ID NOs: 35-41, or SEQ ID NO: 43. In various embodiments, the guanine oxidase comprises an amino acid sequence that is at least 80%, 85%, 90%, 95%, 98%, or 99% identical to the amino acid sequence of any one of SEQ ID NOs: 5-8, SEQ ID NO: 10, SEQ ID NOs: 15-20, SEQ ID NOs: 35-41, or SEQ ID NO: 43. In particular embodiments, the guanine oxidase comprises any one of the amino acid sequences of SEQ ID NO: 5, SEQ ID NO: 19, SEQ ID NO: 20, SEQ ID NO: 35, SEQ ID NO: 36, SEQ ID NO: 38, SEQ ID NO: 39, SEQ ID NO: 40, or SEQ ID NO: 41. In certain embodiments, a variant of the wild-type guanine oxidase is produced by evolving an oxidase enzyme using a directed evolution methodology. In certain embodiments, the directed evolution methodology comprises phage assisted continuous evolution (PACE).

In some embodiments, any of the base editors comprising a guanine oxidase provided herein may further comprise one or more inhibitors of 8-oxoguanine glycosylase (OGG) domain. Without wishing to be bound by any particular theory, the OGG inhibitor domain may inhibit or prevent base excision repair of a oxidized guanine residue, which may improve the activity or efficiency of the base editor.

In various embodiments, the fusion protein further comprises an 8-oxoguanine glycosylase (OGG) inhibitor. In certain embodiments, the OGG inhibitor binds to 8-oxoguanine (8-oxo-G) and may comprise a catalytically inactive OGG enzyme. In various embodiments, the base editors described herein may comprise any of the following structures: NH₂-[napDNAbp]-[guanine oxidase]-COOH; NH₂-[guanine oxidase]-[napDNAbp]-COOH; NH₂-[OGG inhibitor]-[napDNAbp]-[guanine oxidase]-COOH; NH₂-[napDNAbp]-[OGG inhibitor]-[guanine oxidase]-COOH; NH₂-[napDNAbp]-[guanine oxidase]-[OGG inhibitor]-COOH; NH₂-[OGG inhibitor]-[guanine oxidase]-[napDNAbp]-COOH; NH₂-[guanine oxidase]-[OGG inhibitor]-[napDNAbp]-COOH; or NH₂-[guanine oxidase]-[napDNAbp]-[OGG inhibitor]-COOH; wherein each instance of “]-[” comprises an optional linker.

Exemplary guanine oxidase domains include variants with at least 80%, at least 85%, at least 90%, at least 95% or at least 99% sequence identity to the following wild-type enzymes:

S. cyanogenus XDH (“scXDH”):
(SEQ ID NO: 5)
MSHLSERPEKPVVGVSMPHESAVQHVTGAALYTDDLVQRTKDVLHAYPVQVMKARGRVTALRTGAALAVPGVVRVLTGAD
VPGVNDAGMKHDEPLFPDEVMFHGHAVAWVLGETLEAARIGAAAVEVDLEELPSVITLQDAIAADSYHGARPVMTHGDVD
AGFADSAHVFTGEFQFSGQEHFYLETHAALAQVDENGQVFIQSSTQHPSETQEIVSHVLGVPAHEVTVQCLRMGGGFGGK
EMQPHGFAAIAALGAKLTGRPVRFRLNRTQDLTMSGKRHGFHATWKIGFDTEGRIQALDATLTADGGWSLDLSEPVLARA
LCHIDNTYWIPNARVAGRIARTNTVSNTAFRGFGGPQGMLVIEDILGRCAPRLGVDAKELRERNFYRPGQGQTTPYGQPV
TQPERIAAVWQQVQDNGHIADREREIAAFNAAHPHTKRALAVTGVKFGISFNLTAFNQGGALVLIYKDGSVLINHGGTEM
GQGLHTKMLQVAATTLGIPLHKVRLAPTRTDKVPNTSATAASSGADLNGGAVKNACEQLRERLLRVAASQLGTNASDVRI
VEGVARSLGSDQELAWDDLVRTAYFQRVQLSAAGYYRTEGLHWDAKSFRGSPFKYFAIGAAATEVEVDGFTGAYRIRRVD
IVHDVGDSLSPLIDIGQVEGGFVQGAGWLTLEDLRWDTGDGPNRGRLLTQAASTYKLPSFSEMPEEFNVTLLENATEEGA
VFGSKAVGEPPLMLAFSVREALRQAAAAFGPRGTAVELASPATPEAVYWAIESARQGGTAGDGRTHGAAASDAVAVRTGV
EALSGA
C. capitata XDH:
(SEQ ID NO: 6)
MTTNGNSFIVPVEKESPLIFFVNGKKVIDPTPDPECTLLTYLREKLRLCGTKLGCGEGGCGACTVMLSRVDRATNSVKHL
AVNACLMPVCAMHGCAVTTIEGIGSTRTRLHPVQERLAKAHGSQCGFCTPGIVMSMYALLRSMPLPSMKDLEVAFQGNLC
RCTGYRPILEGYKTFTKEFSCGMGEKCCKLQSNGNDVEKNGDDKLFERSAFLPFDPSQEPIFPPELHLNSQFDAENLLFK
GPRSTWYRPVELSDLLKLKSENPHGKIIVGNTEVGVEMKFKQFLYTVHINPIKVPELNEMQELEDSILFGSAVTLMDIEE
YLRERIAKLPEHETRFFRCAVKMLHYFAGKQIRNVASLGGNIMTGSPISDMNPILTAACAKLKVCSLVEGRIETREVCMG
PGFFTGYRKNTIQPHEVLVAIHFPKSKKDQHFVAFKQARRRDDDIAIVNAAVNVTFESNTNIVRQIYMAFGGMAPTTVMV
PKTSQIMAKQKWNRVLVERVSESLCAELPLAPTAPGGMIAYRRSLVVSLFFKAYLAISQELVKSNVIEEDAIPEREQSGA
ATHTPILKSAQLFERVCVEQSTCDPIGRPKVHASAFKQATGEAIYCDDIPRHENELYLALVLSTKAHAKIVSVDESDALK
QAGVHAFFSSKDITEYENKVGSVFHDEEVFASERVYCQGQVIGAIVADSQVLAQRAARLVHIKYEELTPVIITIEQAIKH
KSYFPNYPQYIVQGDVATAFEEADHVYENSCRMGGQEHFYLETNACVATPRDSDEIELFCSTQNPTEVQKLVAHVLSVPC
HRVVCRSKRLGGGFGGKESRSIILALPVALASYRLRRPVRCMLDRDEDMMTTGTRHPFLFKYKVGFTKEGLITACDIECY
NNAGCSMDLSFSVLDRAMNHFENCYRIPNVKVAGWVCRTNLPSNTAFRGFGGPQGMFAAEHIVRDVARIVGKDYLDIMQM
NFYKTGDYTHYNQKLENFPIEKCFTDCLNQSEFHKKRLAIEEFNKKNRWRKRGIALVPTKYGIAFGAMHLNQAGALINIY
GDGSVLLSHGGVEIGQGLHTKMIQCCARALGIPTELIHIAETATDKVPNTSPTAASVGSDINGMAVLDACEKLNQRLKPI
REANPKATWQECISKAYFDRISLSASGFYKMPDVGDDPKTNPNARTYNYFTNGVGVSVVEIDCLTGDHQVLSTDIVMDIG
SSLNPAIDIGQIEGAFMQGYGLFVLEELIYSPQGALYSRGPGMYKLPGFADIPGEFNVSLLTGAPNPRAVYSSKAVGEPP
LFIGSTVFFAIKQAIAAARAERGLSITFELDAPATAARIRMACQDEFTDLIEQPSPGTYTPWNVVP
N. crassa XDH:
(SEQ ID NO: 7)
MTTNGNSFIVPVEKESPLIFFVNGKKVIDPTPDPECTLLTYLREKLRLCGTKLGCGEGGCGACTVMLSRVDRATNSVKHL
AVNACLMPVCAMHGCAVTTIEGIGSTRTRLHPVQERLAKAHGSQCGFCTPGIVMSMYALLRSMPLPSMKDLEVAFQGNLC
RCTGYRPILEGYKTFTKEFSCGMGEKCCKLQSNGNDVEKNGDDKLFERSAFLPFDPSQEPIFPPELHLNSQFDAENLLFK
GPRSTWYRPVELSDLLKLKSENPHGKIIVGNTEVGVEMKFKQFLYTVHINPIKVPELNEMQELEDSILFGSAVTLMDIEE
YLRERIAKLPEHETRFFRCAVKMLHYFAGKQIRNVASLGGNIMTGSPISDMNPILTAACAKLKVCSLVEGRIETREVCMG
PGFFTGYRKNTIQPHEVLVAIHFPKSKKDQHFVAFKQARRRDDDIAIVNAAVNVTFESNTNIVRQIYMAFGGMAPTTVMV
PKTSQIMAKQKWNRVLVERVSESLCAELPLAPTAPGGMIAYRRSLVVSLFFKAYLAISQELVKSNVIEEDAIPEREQSGA
ATHTPILKSAQLFERVCVEQSTCDPIGRPKVHASAFKQATGEAIYCDDIPRHENELYLALVLSTKAHAKIVSVDESDALK
QAGVHAFFSSKDITEYENKVGSVFHDEEVFASERVYCQGQVIGAIVADSQVLAQRAARLVHIKYEELTPVIITIEQAIKH
KSYFPNYPQYIVQGDVATAFEEADHVYENSCRMGGQEHFYLETNACVATPRDSDEIELFCSTQNPTEVQKLVAHVLSVPC
HRVVCRSKRLGGGFGGKESRSIILALPVALASYRLRRPVRCMLDRDEDMMTTGTRHPFLFKYKVGFTKEGLITACDIECY
NNAGCSMDLSFSVLDRAMNHFENCYRIPNVKVAGWVCRTNLPSNTAFRGFGGPQGMFAAEHIVRDVARIVGKDYLDIMQM
NFYKTGDYTHYNQKLENFPIEKCFTDCLNQSEFHKKRLAIEEFNKKNRWRKRGIALVPTKYGIAFGAMHLNQAGALINIY
GDGSVLLSHGGVEIGQGLHTKMIQCCARALGIPTELIHIAETATDKVPNTSPTAASVGSDINGMAVLDACEKLNQRLKPI
REANPKATWQECISKAYFDRISLSASGFYKMPDVGDDPKTNPNARTYNYFTNGVGVSVVEIDCLTGDHQVLSTDIVMDIG
SSLNPAIDIGQIEGAFMQGYGLFVLEELIYSPQGALYSRGPGMYKLPGFADIPGEFNVSLLTGAPNPRAVYSSKAVGEPP
LFIGSTVFFAIKQAIAAARAERGLSITFELDAPATAARIRMACQDEFTDLIEQPSPGTYTPWNVVP
M. Hansupus XDH:
(SEQ ID NO: 8)
MSNMFEFRLNGATVRVDGVSPNTTLLDFLRNRGLTGTKQGCAEGDCGACTVALVDRDAQGNRCLRAFNACIALVPMVAGR
ELVTVEGVGSSEKPHPVQQAMVKHYGSQCGFCTPGFIVSMAEGYSRKDVCTPSSVADQLCGNLCRCTGYRPIRDAMMEAL
AERDADASPATAIPSAPLGGPAEPLSALHYEATGQTFLRPTSWKELLDLRARHPEAHLVAGATELGVDITKKARRFPFLI
STEGVESLREVRREKDCWYVGGAASLVALEEALGDALPEVTKMLNVFASRQIRQRATLAGNLVTASPIGDMAPVLLALDA
RLVLGSVRGERTVALSEFFLAYRKTALQADEVVRHIVIPHPAVPERGQRLSDSFKVSKRRELDISIVAAGFRVELDAHGV
VSLARLGYGGVAATPVRAVRAEAALTGQPWTRETVDQVLPVLAEEITPISDQRGSAEYRRGLVAGLFEKFFAGTYSPVLD
AAPGFEKGDAQVPADAGRALRHESAMGHVTGSARYVDDLAQRQPMLEVWPVCAPHAHARILKRDPTAARKVPGVVRVLMA
EDIPGTNDTGPIRHDEPLLADREVLFHGQIVALVVGESVEACRAGARAVEVEYEPLPAILTVEDAMAQGSYHTEPHVIRR
GDVDAALASSPHRLSGTMAIGGQEHFYLETQAAFAERGDDGDITVVSSTQHPSEVQAIISHVLHLPRSRVVVKSPRMGGG
FGGKETQGNSPAALVALASWHTGRPTRWMMDRDVDMVVTGKRHPFHAAYEVGFDDEGKLLALRVQLVSNGGWSLDLSESI
TDRALFHLDNAYYVPALTYTGRVAKTHLVSNTAFRGFGGPQGMLVTEEVLAHVARSVGVPADVVRERNLYRGTGETNTTH
YGQELEDERIHRVWEELKRTSDFEQRRAEVDAFNARSPFIKRGLAITPMKFGISFTATFLNQAGALVHLYRDGSVMVSHG
GTEMGQGLHTKVQGVAMRELGVEASAVRIAKTATDKVPNTSATAASSGSDLNGAAVRLACITLRERLAPVAVRLLADRHG
RTVAPEALLFSEGKVGLRGEPEVSLPFANVVEAAYLARVGLSATGYYQTPGIGYDKAKGRGRPFLYFAYGASVCEVEVDG
HTGVKRVLRVDLLEDVGDSLNPGVDRGQIEGGFVQGLGWLTGEELRWDANGRLLTHSASTYAVPAFSDAPIDFRVRLLER
AHQHNTIHGSKAVGEPPLMLAMSAREALRDAVGAFGQAGGGVALASPATHEALFLAIQKRLSRGAREDGREAA
E. cloacae XDH:
(SEQ ID NO: 10)
MKFDKPATTNPIDTLRVVGQPHTRIDGPRKTTGSAHYAYEWHDIAPNAAYGHVVGAPIAKGRITAIDTKAAEAAPGVLAV
ITADNAGPLGKGEKNTATLLGGPEIEHYHQAVALVVAETFEQARAAAALVKVTCKRAQGAYDLAAEKASVTEPPEDTPDK
NVGDVATAFASAAVKLDAIYTTPDQSHMAMEPHASMAVWEGDNVTVWTSNQMIDWCRTDLALTLKIPPENVRIVSPYIGG
GFGGKLFLRSDALLAALGARAVKRPVKVMLPRPTIPNNTTHRPATLQHIRIGTDTEGKIVAIAHDSWSGNLPGGTPETAV
QQTELLYAGANRHTGLRLATLDLPEGNAMRAPGEAPGLMALEIAIDEIADKAGVDPVAFRILNDTQVDPANPERRFSRRQ
LVECLQTGAERFGWQKRHAQPGQVRDGRWLVGMGMAAGFRNNLVATSGARVHLNADGSVAVETDMTDIGTGSYTIIAQTA
AEMLGLPLEKVDVRLGDSRFPVSAGSGGQWGANTSTAGVYAACVKLREAIARQLGFDPATAEFADETISAQGRSAPLAEA
AKSGVLTAEDSIEFGDLDKEYQQSTFAGHFVEVGVDSATGEVRVRRMLAVCAAGRILNPITARSQVIGAMTMGLGAALME
ELAVDTRLGYFVNHDMAAYEVPVHAD1PEQEVIFLEDTDPISSPMKAKGVGELGLCGVSAAIANAIYNATGVRVRDYPIT
LDKLIDALPDAV
S. snoursei XDH:
(SEQ ID NO: 15)
MSHDPVPHLPPAAPLPHPLGAPSVRREGREKVTGAARYAAEHTPPGCAYAWPVPATVARGRITELDTAAALALPGVIAVL
THENAPRLASTGDPTLAVLQEDRVPHRGWYVALAVADTLEAARDAAEAVHVGYATEPHDVRITADHPRLYVPEEVFGGPG
ARERGDFDAAFAAAPATVDVAYTVPPLHNHPMEPHAATAQWTDGHLTVHDSSQGATRVCEDLAALFKLGTDEITVVSEHV
GGGFGAKGTPRPQVVLAAMAARHTGRPVKLALPRRQLPGVVGHRAPTLHRVRIGAGHDGVITALAHEIVTHTSTVTEFVE
QAAIPARMMYTSPHSRTVHRLAALDVPTPSWMRAPGEAPGMYALESALDELAVVLDIDPVELRIRNDPATEPDTGRPFSS
RHLVECLRAGAERFGWLPRDPRPAVRRRGDLLLGTGVAAATYPVQISETEAEAHAAADGGYRIRVNATDIGTGARTVLTQ
IAAAVLGAPEDRVRVDIGSSDLPPAVLAGGSTGTASWGWAVHKACTSLLARLRAHHGPLPAEGIMAELSEWAPMALRAWR
IISGLGLPTKYGSTPVALVMRAATEPVAGSGPSVEGPVSSGLVAMKRAPFSMSRMALVSASKL
S. albulus XDH:
(SEQ ID NO: 16)
MTPPPTTRTRAMSHPPEEAPFPPGPPPHPLGDPLVRREGREKVTGTARYAAEHTPDGCAYAWPVPATVVRGRITELDTGA
ALALPGVIAVLTHENAPRLAPTGDPTLALLQEDRVPHRGWYVALAVADTLEAARDAAEAVHVSYATEPHDVTLTADHPRL
YVPAEVFGGPGARERGDFDTAFAAAPATVDVTYTVPPLHNHPMEPHAATALWTHGHLTVHDSSQGATRVREDLAALFKLG
QDQITVHSEHVGGGFGSKGTPRPQVVLAAMAARHTGRPVKLALPRRHLPAVVGHRAPTLHRVRLGAGPDGVITALAHEIV
THTSTVAEFVEQAAMPARIMYTSPHSRTVHRLAALDVPTPSWMRAPGEAPGMYALESAVDELAVVLDLDPIDLRIRNEPG
TEPDTGRPFSSRHLVDCLRAGAARFGWSSRDPRPAVRRQGDLLLGTGVAAATYPVQISATDAEAHAAADGTFRVRVNATD
IGTGARTVLAQIAAAALGAPADRVRVEIGSSDLPPAVLAGGSTGTASWGWAVHKACTVLLARLREHRGPLPAEGVTVTED
TRRETEQPSPYSRHAFGAVFAEVQVDTRTGEVRARRLLGQYAAGHILNPRTARSQFVGGMVMGLGMALTEDSALDPVYGD
FTARDLAAYHVPACADVPAIEAHWLDEEDPHLNPMGSKGIGEIGIVGTPAAIGNAVWHATGVRLRDLPLTPDRILTARTV
PLT
S. himastatinicus XDH:
(SEQ ID NO: 17)
MTRVDGLDKVTGAATYAYEFPTPDVGYVWPVQATIARGRVTEVDGAPALARPGVLAVLDSGNAPRLNTEAQAGPDLFVLQ
SPEVAYHGQIVAAVVATSLEAAREGAAAVRVSYEQEPHDVVLRFDDERAQVAETVTDGSPGFVEHGDAEGALAAAPVRTE
AMYTTPVEHTSPMEPHATIAAWDEDRLTLYNADQGPFMSSQLLAAVFGLDQGAVEVVAEYIGGGFGSKGIPRSPAVLAAL
AAKHLGRPVKIALTRQQMFQLIPYRAPTIQRIRLGAERDGRLTAIDHEVVQQRSAMAEFADQTGSSTRVMYAAPNIRTTV
KTAPLDVLTPAWFRAPGHTPGMFALESAMDELATELEIDPVELRIRNDTGVDPDSGKPFSSRGLVACLREGAARFDWALR
DPKPGIRREGRWLVGTGVASAHHPDYVFPSSATARAEADGTFTVRVGAVDIGTGGRTALTQLAADALGIPVERLRLEIGR
ASLGPAPFAGGSLGTASWGWAVDKACRALLAELDTYGGAVPDGGLEVRADTTEDVELRASFSRHSFGAHFAQVRVDTDTG
EIRVDRMLGVFAAGRIVNPKTARSQFVGAMTMGLSMALLEIGEVDPVFGDFANHDFAGYHVAANADVPKLEALWLDEQDD
NPNPVRGKGIGELGIVGAAAAVTNAFHHATGQRVRDLPIRVERSREALRAARAEAQKRGPGAAEQGKPVG
S. lividans XDH:
(SEQ ID NO: 18)
MSHLSERPEKPVVGVSMPHESAVQHVTGAALYTDDLVQRTKDVLHAYPVQVMKARGRVTALRTGAALAVPGVVRVLTGAD
VPGVNDAGMKHDEPLFPDEVMFHGHAVAWVLGETLEAARIGAAAVEVDLEELPSVITLQDAIAADSYHGARPVMTHGDVD
AGFADSAHVFTGEFQFSGQEHFYLETHAALAQVDENGQVFIQSSTQHPSETQEIVSHVLGVPAHEVTVQCLRMGGGFGGK
EMQPHGFAAIAALGAKLTGRPVRFRLNRTQDLTMSGKRHGFHATWKIGFDTEGRIQALDATLTADGGWSLDLSEPVLARA
LCHIDNTYWIPNARVAGRIARTNTVSNTAFRGFGGPQGMLVIEDILGRCAPRLGVDAKELRERNFYRPGQGQTTPYGQPV
TQPERIAAVWQQVQDNGHIADREREIAAFNAAHPHTKRALAVTGVKFGISFNLTAFNQGGALVLIYKDGSVLINHGGTEM
GQGLHTKMLQVAATTLGIPLHKVRLAPTRTDKVPNTSATAASSGADLNGGAVKNACEQLRERLLRVAASQLGTNASDVRI
VEGVARSLGSDQELAWDDLVRTAYFQRVQLSAAGYYRTEGLHWDAKSFRGSPFKYFAIGAAATEVEVDGFTGAYRIRRVD
IVHDVGDSLSPLIDIGQVEGGFVQGAGWLTLEDLRWDTGDGPNRGRLLTQAASTYKLPSFSEMPEEFNVTLLENATEEGA
VFGSKAVGEPPLMLAFSVREALRQAAAAFGPRGTAVELASPATPEAVYWAIESARQGGTAGDGRTHGAAASDAVAVRTGV
EALSGA
Cytochrome P1A2 (“CYP1A2”):
(SEQ ID NO: 19)
MLASGMLLVALLVCLTVMVLMSVWQQRKSKGKLPPGPTPLPFIGNYLQLNTEQMYNSLMKISERYGPVFTIHLGPRRVVV
LCGHDAVREALVDQAEEFSGRGEQATFDWVFKGYGVVFSNGERAKQLRRFSIATLRDFGVGKRGIEERIQEEAGFLIDAL
RGTGGANIDPTFFLSRTVSNVISSIVFGDRFDYKDKEFLSLLRMMLGIFQFTSTSTGQLYEMFSSVMKHLPGPQQQAFQL
LQGLEDFIAKKVEHNQRTLDPNSPRDFIDSFLIRMQEEEKNPNTEFYLKNLVMTTLNLFIGGTETVSTTLRYGFLLLMKH
PEVEAKVHEEIDRVIGKNRQPKFEDRAKMPYMEAVIHEIQRFGDVIPMSLARRVKKDTKFRDFFLPKGTEVFPMLGSVLR
DPSFFSNPQDFNPQHFLNEKGQFKKSDAFVPFSIGKRNCFGEGLARMELFLFFTTVMQNFRLKSSQSPKDIDVSPKHVGF
ATIPRNYTMSFLPR
CYP2A6:
(SEQ ID NO: 20)
MLASGMLLVALLVCLTVMVLMSVWQQRKSKGKLPPGPTPLPFIGNYLQLNTEQMYNSLMKISERYGPVFTIHLGPRRVVV
LCGHDAVREALVDQAEEFSGRGEQATFDWVFKGYGVVFSNGERAKQLRRFSIATLRDFGVGKRGIEERIQEEAGFLIDAL
RGTGGANIDPTFFLSRTVSNVISSIVFGDRFDYKDKEFLSLLRMMLGIFQFTSTSTGQLYEMFSSVMKHLPGPQQQAFQL
LQGLEDFIAKKVEHNQRTLDPNSPRDFIDSFLIRMQEEEKNPNTEFYLKNLVMTTLNLFIGGTETVSTTLRYGFLLLMKH
PEVEAKVHEEIDRVIGKNRQPKFEDRAKMPYMEAVIHEIQRFGDVIPMSLARRVKKDTKFRDFFLPKGTEVFPMLGSVLR
DPSFFSNPQDFNPQHFLNEKGQFKKSDAFVPFSIGKRNCFGEGLARMELFLFFTTVMQNFRLKSSQSPKDIDVSPKHVGF
ATIPRNYTMSFLPR
CYP3A4:
(SEQ ID NO: 35)
MALIPDLAMETWLLLAVSLVLLYLYGTHSHGLFKKLGIPGPTPLPFLGNILSYHKGFCMFDMECHKKYGKVWGFYDGQQP
VLAITDPDMIKTVLVKECYSVFTNRRPFGPVGFMKSAISIAEDEEWKRLRSLLSPTFTSGKLKEMVPIIAQYGDVLVRNL
RREAETGKPVTLKDVFGAYSMDVITSTSFGVNIDSLNNPQDPFVENTKKLLRFDFLDPFFLSITVFPFLIPILEVLNICV
FPREVTNFLRKSVKRMKESRLEDTQKHRVDFLQLMIDSQNSKETESHKALSDLELVAQSIIFIFAGYETTSSVLSFIMYE
LATHPDVQQKLQEEIDAVLPNKAPPTYDTVLQMEYLDMVVNETLRLFPIAMRLERVCKKDVEINGMFIPKGVVVMIPSYA
LHRDPKYWTEPEKFLPERFSKKNKDNIDPYIYTPFGSGPRNCIGMRFALMNMKLALIRVLQNFSFKPCKETQIPLKLSLG
GLLQPEKPVVLKVESRDGTVSGA
TET1:
(SEQ ID NO: 36)
MSRSRHARPSRLVRKEDVNKKKKNSQLRKTTKGANKNVASVKTLSPGKLKQLIQERDVKKKTEPKPPVPVRSLLTRAGAA
RMNLDRTEVLFQNPESLTCNGFTMALRSTSLSRRLSQPPLVVAKSKKVPLSKGLEKQHDCDYKILPALGVKHSENDSVPM
QDTQVLPDIETLIGVQNPSLLKGKSQETTQFWSQRVEDSKINIPTHSGPAAEILPGPLEGTRCGEGLFSEETLNDTSGSP
KMFAQDTVCAPFPQRATPKVTSQGNPSIQLEELGSRVESLKLSDSYLDPIKSEHDCYPTSSLNKVIPDLNLRNCLALGGS
TSPTSVIKFLLAGSKQATLGAKPDHQEAFEATANQQEVSDTTSFLGQAFGAIPHQWELPGADPVHGEALGETPDLPEIPG
AIPVQGEVFGTILDQQETLGMSGSVVPDLPVFLPVPPNPIATFNAPSKWPEPQSTVSYGLAVQGAIQILPLGSGHTPQSS
SNSEKNSLPPVMAISNVENEKQVHISFLPANTQGFPLAPERGLFHASLGIAQLSQAGPSKSDRGSSQVSVTSTVHVVNTT
VVTMPVPMVSTSSSSYTTLLPTLEKKKRKRCGVCEPCQQKTNCGECTYCKNRKNSHQICKKRKCEELKKKPSVVVPLEVI
KENKRPQREKKPKVLKADFDNKPVNGPKSESMDYSRCGHGEEQKLELNPHTVENVTKNEDSMTGIEVEKWTQNKKSQLTD
HVKGDFSANVPEAEKSKNSEVDKKRTKSPKLFVQTVRNGIKHVHCLPAETNVSFKKFNIEEFGKTLENNSYKFLKDTANH
KNAMSSVATDMSCDHLKGRSNVLVFQQPGFNCSSlPHSSHSIINHHASIHNEGDQPKTPENIPSKEPKDGSPVQPSLLSL
MKDRRLTLEQVVAIEALTQLSEAPSENSSPSKSEKDEESEQRTASLLNSCKAILYTVRKDLQDPNLQGEPPKLNHCPSLE
KQSSCNTVVFNGQTTTLSNSHINSATNQASTKSHEYSKVTNSLSLFlPKSNSSKIDTNKSIAQGIITLDNCSNDLHQLPP
RNNEVEYCNQLLDSSKKLDSDDLSCQDATHTQIEEDVATQLTQLASIIKINYIKPEDKKVESTPTSLVTCNVQQKYNQEK
GTIQQKPPSSVHNNHGSSLTKQKNPTQKKTKSTPSRDRRKKKPTVVSYQENDRQKWEKLSYMYGTICDIWIASKFQNFGQ
FCPHDFPTVFGKISSSTKIWKPLAQTRSIMQPKTVFPPLTQIKLQRYPESAEEKVKVEPLDSLSLFHLKTESNGKAFTDK
AYNSQVQLTVNANQKAHPLTQPSSPPNQCANVMAGDDQIRFQQVVKEQLMHQRLPTLPGISHETPLPESALTLRNVNVVC
SGGITVVSTKSEEEVCSSSFGTSEFSTVDSAQKNFNDYAMNFFTNPTKNLVSITKDSELPTCSCLDRVIQKDKGPYYTHL
GAGPSVAAVREIMENRYGQKGNAlRIEIVVYTGKEGKSSHGCPIAKWVLRRSSDEEKVLCLVRQRTGHHCPTAVMVVLIM
VWDGlPLPMADRLYTELTENLKSYNGHPTDRRCTLNENRTCTCQGIDPETCGASFSFGCSWSMYFNGCKFGRSPSPRRFR
IDPSSPLHEKNLEDNLQSLATRLAPIYKQYAPVAYQNQVEYENVARECRLGSKEGRPFSGVTACLDFCAHPHRDIHNMNN
GSTVVCTLTREDNRSLGVIPQDEQLHVLPLYKLSDTDEFGSKEGMEAKIKSGAIEVLAPRRKKRTCFTQPVPRSGKKRAA
MMTEVLAHKIRAVEKKPIPRIKRKNNSTTTNNSKPSSLPTLGSNTETVQPEVKSETEPHFILKSSDNTKTYSLMPSAPHP
VKEASPGFSWSPKTASATPAPLKNDATASCGFSERSSTPHCTMPSGRLSGANAAAADGPGISQLGEVAPLPTLSAPVMEP
LINSEPSTGVTEPLTPHQPNHQPSFLTSPQDLASSPMEEDEQHSEADEPPSDEPLSDDPLSPAEEKLPHIDEYWSDSEHI
FLDANIGGVAIAPAHGSVLIECARRELHATTPVEHPNRNHPTRLSLVFYQHKNLNKPQHGFELNKIKFEAKEAKNKKMKA
SEQKDQAANEGPEQSSEVNELNQIPSHKALTLTHDNVVTVSPYALTHVAGPYNHWV
TET1-CD (“Catalytic domain”):
(SEQ ID NO: 37)
MGSLPTCSCLDRVIQKDKGPYYTHLGAGPSVAAVREIMENRYGQKGNAIRIEIVVYTGKEGKSSHGCPIAKWVLRRSSDE
EKVLCLVRQRTGHHCPTAVMVVLIMVWDGIPLPMADRLYTELTENLKSYNGHPTDRRCTLNENRTCTCQGIDPETCGASF
SFGCSWSMYFNGCKFGRSPSPRRFRlDPSSPLHEKNLEDNLQSLATRLAPIYKQYAPVAYQNQVEYENVARECRLGSKEG
RPFSGVTACLDFCAHPHRDIHNMNNGSTVVCTLTREDNRSLGVIPQDEQLHVLPLYKLSDTDEFGSKEGMEAKIKSGAIE
VLAPRRKKRTCFTQPVPRSGKKRAAMMTEVLAHKIRAVEKKPIPRIKRKNNSTTTNNSKPSSLPTLGSNTETVQPEVKSE
TEPHFILKSSDNTKTYSLMPSAPHPVKEASPGFSWSPKTASATPAPLKNDATASCGFSERSSTPHCTMPSGRLSGANAAA
ADGPGISQLGEVAPLPTLSAPVMEPLINSEPSTGVTEPLTPHQPNHQPSFLTSPQDLASSPMEEDEQHSEADEPPSDEPL
SDDPLSPAEEKLPHIDEYWSDSEHIFLDANIGGVAIAPAHGSVLIECARRELHATTPVEHPNRNHPTRLSLVFYQHKNLN
KPQHGFELNKIKFEAKEAKNKKMKASEQKDQAANEGPEQSSEVNELNQIPSHKALTLTHDNVVTVSPYALTHVAGPYNHW
V
TET2:
(SEQ ID NO: 38)
MEQDRTNHVEGNRLSPFLIPSPPICQTEPLATKLQNGSPLPERAHPEVNGDTKWHSFKSYYGIPCMKGSQNSRVSPDFTQ
ESRGYSKCLQNGGIKRTVSEPSLSGLLQIKKLKQDQKANGERRNFGVSQERNPGESSQPNVSDLSDKKESVSSVAQENAV
KDFTSFSTHNCSGPENPELQILNEQEGKSANYHDKNIVLLKNKAVLMPNGATVSASSVEHTHGELLEKTLSQYYPDCVSI
AVQKTTSHINAINSQATNELSCEITHPSHTSGQINSAQTSNSELPPKPAAVVSEACDADDADNASKLAAMLNTCSFQKPE
QLQQQKSVFEICPSPAENNIQGTTKLASGEEFCSGSSSNLQAPGGSSERYLKQNEMNGAYFKQSSVFTKDSFSATTTPPP
PSQLLLSPPPPLPQVPQLPSEGKSTLNGGVLEEHHHYPNQSNTTLLREVKIEGKPEAPPSQSPNPSTHVCSPSPMLSERP
QNNCVNRNDIQTAGTMTVPLCSEKTRPMSEHLKHNPPIFGSSGELQDNCQQLMRNKEQEILKGRDKEQTRDLVPPTQHYL
KPGWIELKAPRFHQAESHLKRNEASLPSILQYQPNLSNQMTSKQYTGNSNMPGGLPRQAYTQKTTQLEHKSQMYQVEMNQ
GQSQGTVDQHLQFQKPSHQVHFSKTDHLPKAHVQSLCGTRFHFQQRADSQTEKLMSPVLKQHLNQQASETEPFSNSHLLQ
HKPHKQAAQTQPSQSSHLPQNQQQQQKLQIKNKEEILQTFPHPQSNNDQQREGSFFGQTKVEECFHGENQYSKSSEFETH
NVQMGLEEVQNINRRNSPYSQTMKSSACKIQVSCSNNTHLVSENKEQTTHPELFAGNKTQNLHHMQYFPNNVIPKQDLLH
RCFQEQEQKSQQASVLQGYKNRNQDMSGQQAAQLAQQRYLIHNHANVFPVPDQGGSHTQTPPQKDTQKHAALRWHLLQKQ
EQQQTQQPQTESCHSQMHRPIKVEPGCKPHACMHTAPPENKTWKKVTKQENPPASCDNVQQKSIIETMEQHLKQFHAKSL
FDHKALTLKSQKQVKVEMSGPVTVLTRQTTAAELDSHTPALEQQTTSSEKTPTKRTAASVLNNFIESPSKLLDTPIKNLL
DTPVKTQYDFPSCRCVEQIIEKDEGPFYTHLGAGPNVAAIREIMEERFGQKGKAIRIERVIYTGKEGKSSQGCPIAKWVV
RRSSSEEKLLCLVRERAGHTCEAAVIVILILVWEGIPLSLADKLYSELTETLRKYGTLTNRRCALNEERTCACQGLDPET
CGASFSFGCSWSMYYNGCKFARSKIPRKFKLLGDDPKEEEKLESHLQNLSTLMAPTYKKLAPDAYNNQIEYEHRAPECRL
GLKEGRPFSGVTACLDFCAHAHRDLHNMQNGSTLVCTLTREDNREFGGKPEDEQLHVLPLYKVSDVDEFGSVEAQEEKKR
SGAIQVLSSFRRKVRMLAEPVKTCRQRKLEAKKAAAEKLSSLENSSNKNEKEKSAPSRTKQTENASQAKQLAELLRLSGP
VMQQSQQPQPLQKQPPQPQQQQRPQQQQPHHPQTESVNSYSASGSTNPYMRRPNPVSPYPNSSHTSDIYGSTSPMNFYST
SSQAAGSYLNSSNPMNPYPGLLNQNTQYPSYQCNGNLSVDNCSPYLGSYSPQSQPMDLYRYPSQDPLSKLSLPPIHTLYQ
PRFGNSQSFTSKYLGYGNQNMQGDGFSSCTIRPNVHHVGKLPPYPTHEMDGHFMGATSRLPPNLSNPNMDYKNGEHHSPS
HIIHNYSAAPGMFNSSLHALHLQNKENDMLSHTANGLSKMLPALNHDRTACVQGGLHKLSDANGQEKQPLALVQGVASGA
EDNDEVWSDSEQSFLDPDIGGVAVAPTHGSILIECAKRELHATTPLKNPNRNHPTRISLVFYQHKSMNEPKHGLALWEAK
MAEKAREKEEECEKYGPDYVPQKSHGKKVKREPAEPHETSEPTYLRFIKSLAERTMSVTTDSTVTTSPYAFTRVTGPYNR
YI
TET3:
(SEQ ID NO: 39)
MDSGPVYHGDSRQLSASGVPVNGAREPAGPSLLGTGGPWRVDQKPDWEAAPGPAHTARLEDAHDLVAFSAVAEAVSSYGA
LSTRLYETFNREMSREAGNNSRGPRPGPEGCSAGSEDLDTLQTALALARHGMKPPNCNCDGPECPDYLEWLEGKIKSVVM
EGGEERPRLPGPLPPGEAGLPAPSTRPLLSSEVPQISPQEGLPLSQSALSIAKEKNISLQTAIAIEALTQLSSALPQPSH
STPQASCPLPEALSPPAPFRSPQSYLRAPSWPVVPPEEHSSFAPDSSAFPPATPRTEFPEAWGTDTPPATPRSSWPMPRP
SPDPMAELEQLLGSASDYIQSVFKRPEALPTKPKVKVEAPSSSPAPAPSPVLQREAPTPSSEPDTHQKAQTALQQHLHHK
RSLFLEQVHDTSFPAPSEPSAPGWWPPPSSPVPRLPDRPPKEKKKKLPTPAGGPVGTEKAAPGIKPSVRKPIQIKKSRPR
EAQPLFPPVRQIVLEGLRSPASQEVQAHPPAPLPASQGSAVPLPPEPSLALFAPSPSRDSLLPPTQEMRSPSPMTALQPG
STGPLPPADDKLEELIRQFEAEFGDSFGLPGPPSVPIQDPENQQTCLPAPESPFATRSPKQIKIESSGAVTVLSTTCFHS
EEGGQEATPTKAENPLTPTLSGFLESPLKYLDTPTKSLLDTPAKRAQAEFPTCDCVEQIVEKDEGPYYTHLGSGPTVASI
RELMEERYGEKGKAIRIEKVIYTGKEGKSSRGCPIAKWVIRRHTLEEKLLCLVRHRAGHHCQNAVIVILILAWEGIPRSL
GDTLYQELTDTLRKYGNPTSRRCGLNDDRTCACQGKDPNTCGASFSFGCSWSMYFNGCKYARSKTPRKFRLAGDNPKEEE
VLRKSFQDLATEVAPLYKRLAPQAYQNQVTNEEIAIDCRLGLKEGRPFAGVTACMDFCAHAHKDQHNLYNGCTVVCTLTK
EDNRCVGKIPEDEQLHVLPLYKMANTDEFGSEENQNAKVGSGAIQVLTAFPREVRRLPEPAKSCRQRQLEARKAAAEKKK
IQKEKLSTPEKIKQEALELAGITSDPGLSLKGGLSQQGLKPSLKVEPQNHFSSFKYSGNAVVESYSVLGNCRPSDPYSMN
SVYSYHSYYAQPSLTSVNGFHSKYALPSFSYYGFPSSNPVFPSQFLGPGAWGHSGSSGSFEKKPDLHALHNSLSPAYGGA
EFAELPSQAVPTDAHHPTPHHQQPAYPGPKEYLLPKAPLLHSVSRDPSPFAQSSNCYNRSIKQEPVDPLTQAEPVPRDAG
KMGKTPLSEVSQNGGPSHLWGQYSGGPSMSPKRTNGVGGSWGVFSSGESPAIVPDKLSSFGASCLAPSHFTDGQWGLFPG
EGQQAASHSGGRLRGKPWSPCKFGNSTSALAGPSLTEKPWALGAGDFNSALKGSPGFQDKLWNPMKGEEGRIPAAGASQL
DRAWQSFGLPLGSSEKLFGALKSEEKLWDPFSLEEGPAEEPPSKGAVKEEKGGGGAEEEEEELWSDSEHNFLDENIGGVA
VAPAHGSILIECARRELHATTPLKKPNRCHPTRISLVFYQHKNLNQPNHGLALWEAKMKQLAERARARQEEAARLGLGQQ
EAKLYGKKRKWGGTVVAEPQQKEKKGVVPTRQALAVPTDSAVTVSSYAYTKVTGPYSRWI
E. coli AlkB:
(SEQ ID NO: 40)
MLDLFADAEPWQEPLAAGAVILRRFAFNAAEQLIRDINDVASQSPFRQMVTPGGYTMSVAMTNCGHLGWTTHRQGYLYSP
IDPQTNKPWPAMPQSFHNLCQRAATAAGYPDFQPDACLINRYAPGAKLSLHQDKDEPDLRAPIVSVSLGLPAIFQFGGLK
RNDPLKRLLLEHGDVVVWGGESRLFYHGIQPLKAGFHPLTIDCRYNLTFRQAGKKE
ABH3 (human):
(SEQ ID NO: 41)
MEEKRRRARVQGAWAAPVKSQAIAQPATTAKSHLHQKPGQTWKNKEHHLSDREFVFKEPQQVVRRAPEPRVIEEGVYEIS
LSPTGVSRVCLYPGFVDVKEADWILEQLCQDVPWKQRTGIREDSILQLTFKKSAPVSGTATAPQSCWYERPSPPHIPGPA
ILTRTRLWAP
E. coli GMP Synthase:
(SEQ ID NO: 43)
MTENIHKHRILILDFGSQYTQLVARRVRELGVYCELWAWDVTEAQIRDFNPSGIILSGGPESTTEENSPRAPQYVFEAGV
PVFGVCYGMQTMAMQLGGHVEASNEREFGYAQVEVVNDSALVRGIEDALTADGKPLLDVWMSHGDKVTAIPSDFITVAST
ESCPFAIMANEEKRFYGVQFHPEVTHTRQGMRMLERFVRDICQCEALWTPAKIIDDAVARIREQVGDDKVILGLSGGVDS
SVTAMLLHRAIGKNLTCVFVDNGLLRLNEAEQVLDMFGDHFGLNIVHVPAEDRFLSALAGENDPEAKRKIIGRVFVEVFD
EEALKLEDVKWLAQGTIYPDVIESAASATGKAHVIKSHHNVGGLPKEMKMGLVEPLKELFKDEVRKIGLELGLPYDMLYR
HPFPGPGLGVRVLGEVKKEYCDLLRRADAIFIEELRKADLYDKVSQAFTVFLPVRSVGVMGDGRKYDWVVSLRAVETIDF
MTAHWAHLPYDFLGRVSNRIINEVNGISRVVYDISGKPPATIEWE

(C) Guanine Methyltransferases

In various embodiments, the GTBE (and CABE) base editors provided herein comprise a guanine methyltransferase nucleobase modification domain (FIG. 1B). Any methyltransferase that is adapted to accept guanine nucleotide substrates are useful in the base editors and methods of editing disclosed herein. A guanine methyltransferase is an enzyme that catalyzes the alkylation (with a methyl group) of a guanine nucleobase to form a N₂,N₂-dimethyl-guanine and/or N₁-methyl-guanine (see FIG. 2B). The guanine methyltransferase may comprise a naturally-occurring or modified alkyl transferase, such as an alkyltransferase engineered from a reference enzyme such as ribosomal RNA alkyltransferase RlmA. Modified oxidases may be obtained by, e.g., evolving a reference alkyltransferase (e.g., an rRNA modification enzyme) evolved using a continuous evolution process (e.g., PACE) or non-continuous evolution process (e.g., PANCE or plate-based selections) described herein so that the alkyltransferase is effective on a nucleic acid target.

In certain embodiments, the guanine methyltransferase is a wild-type RlmA, or a variant thereof, that methylates a guanine in DNA. In certain embodiments, the RlmA is a Escherichia coli RlmA, or a variant thereof.

In one embodiment, the guanine methyltransferase is a dimethyltransferase that methylates a guanine to N₂,N₂-dimethylguanine. In various embodiments, the dimethyltransferase is a Trm1, or a variant thereof, that methylates a guanine in DNA. In other embodiments, the dimethyltransferase is a Aquifex aeolicus Trm1 or variant thereof. In certain embodiments, the dimethyltransferase is a human Trm1 or variant thereof. In certain embodiments, the dimethyltransferase is a Saccharomyces cerevisiae Trm1 or variant thereof.

In one embodiment, the guanine methyltransferase methylates a guanine to N₁-methyl-guanine. In various embodiments, the methyltransferase is a RlmA, a TrmT10A, a TrmD, or variants thereof, that methylates a guanine in DNA. In various embodiments, the methyltransferase is an Escherichia coli RlmA, human TrmT10A, Escherichia coli TrmD, M. jannaschii Trm5b, P. abyssi Trm5a or the Trm5c of a suitable archaeon. In certain embodiments, the methyltransfease is an Escherichia coli TrmD having one or more of the following mutations: M149V, G189V, and E194K.

In other embodiments, the guanine methyltransferase methylates a guanine to 8-methyl-guanine. In certain embodiments, the guanine methyltransferase is a wild-type Cfr, or a variant thereof, that methylates a guanine in DNA. The cell recognizes the mismatch between 8-methyl-G and the cytosine on the unmutated strand and repairs the cytosine to an adenine. Upon a subsequent round of replication, the 8-methyl-G is converted to a thymine. In certain embodiments, the Cfr is a Staphylococcus scirui Cfr, or a variant thereof.

In various embodiments, the guanine methyltransferase comprises any one of the amino acid sequences of SEQ ID NO: 44 or SEQ ID NOs: 46-53. In various embodiments, the guanine methyltransferase comprises an amino acid sequence that is at least 80%, 85%, 90%, 95%, 98%, or 99% identical to the amino acid sequence of any one of SEQ ID NO: 44 or SEQ ID NOs: 46-53. In particular embodiments, the guanine methyltransferase comprises any one of the amino acid sequences of SEQ ID NO: 44, SEQ ID NO: 49, SEQ ID NO: 50, or SEQ ID NO: 51. In certain embodiments, a variant of the wild-type guanine oxidase is produced by evolving a methyltransferase enzyme by a methodology for directed evolution. In certain embodiments, the evolving includes phage assisted continuous evolution (PACE). In other embodiments, the evolving includes phage assisted non-continuous evolution (PANCE).

In certain embodiments, any of the base editors comprising a guanine methyltransferase described herein may further comprise an alkylation lesion repair enzyme inhibitor (“ALRE inhibitor”). In certain embodiments, the ALRE inhibitor binds to N₂,N₂-dimethyl-guanine and/or N₁-methyl-guanine and may comprise a catalytically inactive ALRE that binds N₂,N₂-dimethyl-guanine and/or N₁-methyl-guanine to prevent its excision during subsequent mismatch repair.

In various embodiments, the base editor fusion proteins described herein may comprise any of the following structures: NH₂-[napDNAbp]-[guanine methyltransferase]-COOH; or NH₂-[guanine methyltransferase]-[napDNAbp]-COOH; wherein each instance of “H” comprises an optional linker.

In various embodiments, the base editors described herein may comprise any of the following structures: NH₂-[napDNAbp]-[guanine methyltransferase]-COOH; NH₂-[guanine methyltransferase]-[napDNAbp]-COOH; NH₂-[ALRE inhibitor]-[napDNAbp]-[guanine oxidase]-COOH; NH₂-[napDNAbp]-[ALRE inhibitor]-[guanine oxidase]-COOH; NH₂-[napDNAbp]-[guanine oxidase]-[ALRE inhibitor]-COOH; NH₂-[ALRE inhibitor]-[guanine oxidase]-[napDNAbp]-COOH; NH₂-[guanine oxidase]-[ALRE inhibitor]-[napDNAbp]-COOH; or NH₂-[guanine oxidase]-[napDNAbp]-[ALRE inhibitor]-COOH; wherein each instance of “]-[” comprises an optional linker.

In still another embodiment, the guanine methyltransferase methylates a guanine to 8-methyl-guanine. In certain embodiments, the guanine methyltransferase is a wild-type Cfr, or a variant thereof, that methylates a guanine in DNA. In certain embodiments, the Cfr is a Staphylococcus scirui Cfr, or a variant thereof.

Some exemplary suitable nucleobase modification domains, e.g., guanine methyltransferase domains, that can be fused to Cas9 domains according to embodiments of this disclosure are provided below. Exemplary guanine methyltransferase domains include:

S. scirui Cfr:
(SEQ ID NO: 44)
MNFNNKTKYGKIQEFLRSNNEPDYRIKQITNAIFKQRISRFEDMKVLPKL
LREDLINNFGETVLNIKLLAEQNSEQVTKVLFEVSKNERVETVNMKYKAG
WESFCISSQCGCNFGCKFCATGDIGLKKNLTVDEITDQVLYFHLLGHQID
SISFMGMGEALANRQVFDALDSFTDPNLFALSPRRLSISTIGIIPSIKKI
TQEYPQVNLTFSLHSPYSEERSKLMPINDRYPIDEVMNILDEHIRLTSRK
VYIAYIMLPGVNDSLEHANEVVSLLKSRYKSGKLYHVNLIRYNPTISAPE
MYGEANEGQVEAFYKVLKSAGIHVTIRSQFGIDIDAACGQLYGNYQNSQ
A. aeolicus Trm1:
(SEQ ID NO: 46)
MEIVQEGIAKIIVPEIPKTVSSDMPVFYNPRMRVNRDLAVLGLEYLCKKL
GRPVKVADPLSASGIRAIRFLLETSCVEKAYANDISSKAIEIMKENFKLN
NIPEDRYEIHGMEANFFLRKEWGFGFDYVDLDPFGTPVPFIESVALSMKR
GGILSLTATDTAPLSGTYPKTCMRRYMARPLRNEFKHEVGIRILIKKVIE
LAAQYDIAMIPIFAYSHLHYFKLFFVKERGVEKVDKLIEQFGYIQYCFNC
MNREVVTDLYKFKEKCPHCGSKFHIGGPLWIGKLWDEEFTNFLYEEAQKR
EEIEKETKRILKLIKEESQLQTVGFYVLSKLAEKVKLPAQPPIRIAVKFF
NGVRTHFVGDGFRTNLSFEEVMKKMEELKEKQKEFLEKKKQG
S. cerevisiae Trm1:
(SEQ ID NO: 47)
MEGFFRIPLKRANLHGMLKAAISKIKANFTAYGAPRINIEDFNIVKEGKA
EILFPKKETVFYNPIQQFNRDLSVTCIKAWDNLYGEECGQKRNNKKSKKK
RCAETNDDSSKRQKMGNGSPKEAVGNSNRNEPYINILEALSATGLRAIRY
AHEIPHVREVIANDLLPEAVESIKRNVEYNSVENIVKPNLDDANVLMYRN
KATNNKFHVIDLDPYGTVTPFVDAAIQSIEEGGLMLVTCTDLSVLAGNGY
PEKCFALYGGANMVSHESTHESALRLVLNLLKQTAAKYKKTVEPLLSLSI
DFYVRVFVKVKTSPIEVKNVMSSTMTTYHCSRCGSYHNQPLGRISQREGR
NNKTFTKYSVAQGPPVDTKCKFCEGTYHLAGPMYAGPLHNKEFIEEVLRI
NKEEHRDQDDTYGTRKRIEGMLSLAKNELSDSPFYFSPNHIASVIKLQVP
PLKKVVAGLGSLGFECSLTHAQPSSLKTNAPWDAIWYVMQKCDDEKKDLS
KMNPNTTGYKILSAMPGWLSGTVKSEYDSKLSFAPNEQSGNIEKLRKLKI
VRYQENPTKNWGPKARPNTS
TRM1 (human):
(SEQ ID NO: 48)
MQGSSLWLSLTFRSARVLSRARFFEWQSPGLPNTAAMENGTGPYGEERPR
EVQETTVTEGAAKIAFPSANEVFYNPVQEFNRDLTCAVITEFARIQLGAK
GIQIKVPGEKDTQKVVVDLSEQEEEKVELKESENLASGDQPRTAAVGEIC
EEGLHVLEGLAASGLRSIRFALEVPGLRSVVANDASTRAVDLIRRNVQLN
DVAHLVQPSQADARMLMYQHQRVSERFDVIDLDPYGSPATFLDAAVQAVS
EGGLLCVTCTDMAVLAGNSGETCYSKYGAMALKSRACHEMALRIVLHSLD
LRANCYQRFVVPLLSISADFYVRVFVRVFTGQAKVKASASKQALVFQCVG
CGAFHLQRLGKASGVPSGRAKFSAACGPPVTPECEHCGQRHQLGGPMWAE
PIHDLDFVGRVLEAVSANPGRFHTSERIRGVLSVITEELPDVPLYYTLDQ
LSSTIHCNTPSLLQLRSALLHADFRVSLSHACKNAVKTDAPASALWDIMR
CWEKECPVKRERLSETSPAFRILSVEPRLQANFTIREDANPSSRQRGLKR
FQANPEANWGPRPRARPGGKAADEAMEERRRLLQNKRKEPPEDVAQRAAR
LKTFPCKRFKEGTCQRGDQCCYSHSPPTPRVSADAAPDCPETSNQTPPGP
GAAAGPGID
E. coli R1mA:
(SEQ ID NO: 49)
MSFSCPLCHQPLSREKNSYICPQRHQFDMAKEGYVNLLPVQHKRSRDPGD
SAEMMQARRAFLDAGHYQPLRDAIVAQLRERLDDKATAVLDIGCGEGYYT
HAFADALPEITTFGLDVSKVAIKAAAKRYPQVTFCVASSHRLPFSDTSMD
AIIRIYAPCKAEELARVVKPGGWVITATPGPRHLMELKGLIYNEVHLHAP
HAEQLEGFTLQQSAELCYPMRLRGDEAVALLQMTPFAWRAKPEVWQTLAA
KEVFDCQTDFNIHLWQRSY
E. coli TrmD:
(SEQ ID NO: 50)
MWIGIISLFPEMFRAITDYGVTGRAVKNGLLSIQSWSPRDFTHDRHRTVD
DRPYGGGPGMLMMVQPLRDAIHAAKAAAGEGAKVIYLSPQGRKLDQAGVS
ELATNQKLILVCGRYEGIDERVIQTEIDEEWSIGDYVLSGGELPAMTLID
SVSRFIPGVLGHEASATEDSFAEGLLDCPHYTRPEVLEGMEVPPVLLSGN
HAEIRRWRLKQSLGRTWLRRPELLENLALTEEQARLLAEFKTEHAQQQHK
HDGMA
TRMT10A (human):
(SEQ ID NO: 51)
MSSEMLPAFIETSNVDKKQGINEDQEESQKPRLGEGCEPISKRQMKKLIK
QKQWEEQRELRKQKRKEKRKRKKLERQCQMEPNSDGHDRKRVRRDVVHST
LRLIIDCSFDHLMVLKDIKKLHKQIQRCYAENRRALHPVQFYLTSHGGQL
KKNMDENDKGWVNWKDIHIKPEHYSELIKKEDLIYLTSDSPNILKELDES
KAYVIGGLVDHNHHKGLTYKQASDYGINHAQLPLGNFVKMNSRKVLAVNH
VFEIILEYLETRDWQEAFFTILPQRKGAVPTDKACESASHDNQSVRMEEG
GSDSDSSEEEYSRNELDSPHEEKQDKENHTESTVNSLPH
M. Jannaschii Trm5b:
(SEQ ID NO: 52)
MPLCLKINKKHGEQTRRILIENNLLNKDYKITSEGNYLYLPIKDVDEDIL
KSILNIEFELVDKELEEKKIIKKPSFREIISKKYRKEIDEGLISLSYDVV
GDLVILQISDEVDEKIRKEIGELAYKLIPCKGVFRRKSEVKGEFRVRELE
HLAGENRTLTIHKENGYRLWVDIAKVYFSPRLGGERARIMKKVSLNDVVV
DMFAGVGPFSIACKNAKKIYAIDINPHAIELLKKNIKLNKLEHKIIPILS
DVREVDVKGNRVIMNLPKFAHKFIDKALDIVEEGGVIHYYTIGKDFDKAI
KLFEKKCDCEVLEKRIVKSYAPREYILALDFKINKK.
P. Abyssi Trm5a:
(SEQ ID NO: 53)
MTLAVKVPLKEGEIVRRRLIELGALDNTYKIKREGNFLLIPVKFPVKGFE
VVEAELEQVSRRPNSYREIVNVPQELRRFLPTSFDIIGNIAIIEIPEELK
GYAKEIGRAIVEVHKNVKAVYMKGSKIEGEYRTRELIHIAGENITETIHR
ENGIRLKLDVAKVYFSPRLATERMRVFKMAQEGEVVFDMFAGVGPFSILL
AKKAELVFACDINPWAIKYLEENIKLNKVNNVVPILGDSREIEVKADRII
MNLPKYAHEFLEHAISCINDGGVIHYYGFGPEGDPYGWHLERIRELANKF
GVKVEVLGKRVIRNYAPRQYNIAIDFRVSF.

(D) Additional Base Editor Elements

In certain embodiments, the base editors disclosed herein further comprise a nuclear localization sequence. In various embodiments, the base editors disclosed herein further comprise one or more, preferably, at least two nuclear localization signals. In certain embodiments, the base editors comprise at least two NLSs. In embodiments with at least two NLSs, the NLSs can be the same NLSs or they can be different NLSs. In addition, the NLSs may be expressed as part of a fusion protein with the other domains of the base editors. The location of the NLS fusion can be at the N-terminus, the C-terminus, or within a sequence of a base editor (e.g., inserted between the napDNAbp domain (e.g., dCas9) and a DNA nucleobase modification domain (e.g., a guanine oxidase)).

A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

The NLSs may be any known NLS in the art. The NLSs may also be any NLSs for nuclear localization discovered in the future. The NLSs also may be any naturally-occurring NLS, or any non-naturally occurring NLS (e.g., an NLS with one or more desired mutations).

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines on the protein surface. Different nuclear localized proteins may share the same NLS. An NLS has the opposite function of a nuclear export signal (NES), which targets proteins out of the nucleus. A nuclear localization signal can also target the exterior surface of a cell. Thus, a single nuclear localization signal can direct the entity with which it is associated to the exterior of a cell and to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, the NLS comprises any one of the amino acid sequences PKKKRKV (SEQ ID NO: 81), MDSLLMNRRKFLYQFKNVRWAKGRRETYLC (SEQ ID NO: 82), KRTADGSEFESPKKKRKV (SEQ ID NO: 84), or KRTADGSEFEPKKKRKV (SEQ ID NO: 13). In other embodiments, the NLS comprises any one of the amino acid sequences NLSKRPAAIKKAGQAKKKK (SEQ ID NO: 54), PAAKRVKLD (SEQ ID NO: 55), RQRRNELKRSF (SEQ ID NO: 56), or NQSSNFGPMKGGNFGGRSSGPYGGGGQYFAKPRNQGGY (SEQ ID NO: 57).

Most NLSs can be classified in three general groups: (i) a monopartite NLS exemplified by the SV40 large T antigen NLS (PKKKRKV (SEQ ID NO: 81)); (ii) a bipartite motif consisting of two basic domains separated by a variable number of spacer amino acids and exemplified by the Xenopus nucleoplasmin NLS (KRXXXXXXXXXXKKKL (SEQ ID NO: 85)); and (iii) noncanonical sequences such as M9 of the hnRNP A1 protein, the influenza virus nucleoprotein NLS, and the yeast Gal4 protein NLS (Dingwall and Laskey, Trends Biochem Sci. 1991 December; 16(12):478-81).

Nuclear localization signals appear at various points in the amino acid sequences of proteins. NLSs have been identified at the N-terminus, the C-terminus and in the central region of proteins. Thus, the specification provides base editors that may be modified with one or more NLSs at the C-terminus, the N-terminus, as well as at in internal region of the base editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a base editor to include one or more NLSs. In one aspect, the base editors can be engineered to express a base editor protein that is translationally fused at its N-terminus or its C-terminus (or both) to one or more NLSs, i.e., to form a base editor-NLS fusion construct. In other embodiments, the base editor-encoding nucleotide sequence can be genetically modified to incorporate a reading frame that encodes one or more NLSs in an internal region of the encoded base editor. In addition, the NLSs may include various amino acid linkers or spacer regions encoded between the base editor and the N-terminally, C-terminally, or internally-attached NLS amino acid sequence. Thus, the present disclosure also provides for nucleotide constructs, vectors, and host cells for expressing fusion proteins that comprise a base editor and one or more NLSs.

The base editors described herein may also comprise nuclear localization signals which are linked to a base editor through one or more linkers, e.g., and polymeric, amino acid, nucleic acid, polysaccharide, chemical, or nucleic acid linker element. In certain embodiments, the NLS is linked to a base editor using an XTEN linker, as set forth in SEQ ID NO: 11. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the base editor by any suitable strategy that effectuates forming a bond (e.g., covalent linkage, hydrogen bonding) between the base editor and the one or more NLSs.

The base editors described herein also may include one or more additional elements. In certain embodiments, an additional element may include an effector of base repair, such as an inhibitor of base repair.

In some embodiments, the base editor described herein may comprise one or more protein domains (e.g., about or more than about 1, 2, 3, 4, 5, 6, 7, 8, 9, 10, or more domains in addition to the base editor components). A base editor may comprise any additional protein sequence, and optionally a linker sequence between any two domains. Examples of protein domains that may be fused to a base editor or component thereof (e.g., the napDNAbp domain, the nucleobase modification domain, or the NLS domain) include, without limitation, epitope tags, and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a base editor are described in US Publication No. 2011/0059502, published Mar. 10, 2011 and incorporated herein by reference.

In an aspect of the invention, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In certain embodiments of the invention the gene product is luciferase. In a further embodiment of the invention the expression of the gene product is decreased.

Other exemplary features that may be present are tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, bgh-PolyA tags, polyhistidine tags, and also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

(E) Linkers

In certain embodiments, linkers may be used to link any of the peptides or peptide domains or domains of the base editor (e.g., a napDNAbp domain covalently linked to a nucleobase modification domain which is covalently linked to an NLS domain).

As defined above, the term “linker,” as used herein, refers to a chemical group or a molecule linking two molecules or domains, e.g., a napDNAbp domain and a cleavage domain of a nuclease. In some embodiments, a linker joins an dCas9 and base editor domain (e.g., a guanine oxidase). Typically, the linker is positioned between, or flanked by, two groups, molecules, or other domains and connected to each one via a covalent bond, thus connecting the two. In some embodiments, the linker is an amino acid or a plurality of amino acids (e.g., a peptide or protein). In some embodiments, the linker is an organic molecule, group, polymer, or chemical domain. Chemical domains include, but are not limited to, disulfide, hydrazone, thiol, amide, ester, carbon-carbon bond, carbon-heteroatom bond, urea, carbamate, and azo domains.

The linker may comprise a peptide or a non-peptide moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is a single atom in length. Longer or shorter linkers are also contemplated.

The linker may be as simple as a covalent bond, or it may be a multi-atom linker or polymeric linker many atoms in length. In certain embodiments, the linker is a polpeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, polyether, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic domain (e.g., cyclopentane, cyclohexane). In other embodiments, the linker comprises a polyethylene glycol domain (PEG). In other embodiments, the linker comprises amino acids. In certain embodiments, the linker comprises a peptide. In certain embodiments, the linker comprises an aryl or heteroaryl domain. In certain embodiments, the linker is based on a phenyl ring. The linker may included functionalized domains to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)n (SEQ ID NO: 93), (G)n (SEQ ID NO: 94), (EAAAK)n (SEQ ID NO: 95), (GGS)n (SEQ ID NO: 96), (SGGS)n (SEQ ID NO: 97), (XP)n (SEQ ID NO: 98), or any combination thereof, wherein n is independently an integer between 1 and 30, and wherein X is any amino acid. In some embodiments, the linker comprises the amino acid sequence (GGS)n (SEQ ID NO: 83), wherein n is 1, 3, or 7. In some embodiments, the linker comprises the amino acid sequence SGSETPGTSESATPES (SEQ ID NO: 48). In some embodiments, the linker comprises the amino acid sequence SGGSSGGSSGSETPGTSESATPESSGGSSGGS (SEQ ID NO: 11), also known as an XTEN linker. In some embodiments, the linker comprises the amino acid sequence SGGSGGSGGS (SEQ ID NO: 12). In some embodiments, the linker comprises the amino acid sequence SGGS (SEQ ID NO: 14).

In some embodiments, the fusion protein comprises the structure [guanine oxidase]-[optional linker sequence]-[dCas9 or Cas9 nickase]-[optional linker sequence], or [dCas9 or Cas9 nickase]-[optional linker sequence]-[guanine oxidase].

In some embodiments, the fusion protein comprises the structure [guanine methyltransferase]-[optional linker sequence]-[dCas9 or Cas9 nickase]-[optional linker sequence], or [dCas9 or Cas9 nickase]-[optional linker sequence]-[guanine methyltransferase].

(F) Guide Sequences (e.g., Guide RNAs)

In various embodiments, the GTBE base editors may be complexed, bound, or otherwise associated with (e.g., via any type of covalent or non-covalent bond) one or more guide sequences. The guide sequence becomes associated or bound to the base editor and directs its localization to a specific target sequence having complementarity to the guide sequence or a portion thereof. The particular design embodiments of a guide sequence will depend upon the nucleotide sequence of a genomic target site of interest (i.e., the desired site to be edited) and the type of napDNAbp (e.g., a Cas9 protein) present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a target sequence.

In some embodiments, a guide sequence is less than about 200, 175, 150, 125, 100, 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence. Similarly, cleavage of a target polynucleotide sequence may be evaluated in situ by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 58) where NNNNNNNNNNNNXGG (N is A, G, T, or C; and X can be anything) (SEQ ID NO: 59) has a single occurrence in the genome. A unique target sequence in a genome may include an S. pyogenes Cas9 target site of the form MMMMMMMMMNNNNNNNNNNNXGG (SEQ ID NO: 60) where NNNNNNNNNNNXGG (N is A, G, T, or C; and X can be anything) (SEQ ID NO: 61) has a single occurrence in the genome. For the S. thermophilus CRISPR/Cas9, a unique target sequence in a genome may include a Cas9 target site of the form MMMMMMMMNNNNNNNNNNNNXXAGAAW (SEQ ID NO: 62) where NNNNNNNNNNNNXXAGAAW (N is A, G, T, or C; X can be anything; and W is A or T) (SEQ ID NO: 63) has a single occurrence in the genome. A unique target sequence in a genome may include an S. thermophilus CRISPR 1 Cas9 target site of the form MMMMMMMMMNNNNNNNNNNNXXAGAAW (SEQ ID NO: 64) where NNNNNNNNNNNXXAGAAW (N is A, G, T, or C; X can be anything; and W is A or T) (SEQ ID NO: 65) has a single occurrence in the genome. For the S. pyogenes Cas9, a unique target sequence in a genome may include a Cas9 target site of the form MMMMMMMMNNNNNNNNNNNNXGGXG (SEQ ID NO: 66) where NNNNNNNNNNNNXGGXG (N is A, G, T, or C; and X can be anything) (SEQ ID NO: 67) has a single occurrence in the genome. A unique target sequence in a genome may include an S. pyogenes Cas9 target site of the form MMMMMMMMMNNNNNNNNNNNXGGXG (SEQ ID NO: 68) where NNNNNNNNNNNXGGXG (N is A, G, T, or C; and X can be anything) (SEQ ID NO: 69) has a single occurrence in the genome. In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and P A Carr & G M Church, 2009, Nature Biotechnology 27(12): 1151-62). Additional algorithms may be found in Chuai, G. et al., DeepCRISPR: optimized CRISPR guide RNA design by deep learning, Genome Biol. 19:80 (2018), and U.S. Application Ser. No. 61/836,080 and U.S. Pat. No. 8,871,445, issued Oct. 28, 2014, each of which are incorporated herein by reference.

The guide sequence is linked to a tracr mate sequence which in turn hybridizes to a tracr sequence. A tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In certain embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator: (1) NNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 86); (2) NNNNNNNNNNNNNNNNNNgtttttgtactctcaGAAAtgcagaagctacaaagataaggcttcatgccgaaatca acaccctgtcattttatggcagggtgttttcgttatttaaTTTTTT (SEQ ID NO: 87); (3) NNNNNNNNNNNNNNNNNNNNgtttttgtactctcaGAAAtgcagaagctacaaagataaggcttcatgccgaa atca acaccctgtcattttatggcagggtgtTTTTT (SEQ ID NO: 88); (4) NNNNNNNNNNNNNNNNNNNNgttttagagctaGAAAtagcaagttaaaataaggctagtccgttatcaacttga aaa agtggcaccgagtcggtgcTTTTTT (SEQ ID NO: 89); (5) NNNNNNNNNNNNNNNNNNNNgttttagagctaGAAATAGcaagttaaaataaggctagtccgttatcaactt gaa aaagtgTTTTTTT (SEQ ID NO: 90); and (6) NNNNNNNNNNNNNNNNNNNNgttttagagctagAAATAGcaagttaaaataaggctagtccgttatcaTTT TT TTT (SEQ ID NO: 91). In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and a guanine oxidase, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, the guide RNA comprises a structure 5′-[guide sequence]-guuuuagagcuagaaauagcaaguuaaaauaaggcuaguccguuaucaacuugaaaaaguggcaccgagucggugcuuu uu-3′ (SEQ ID NO: 92), wherein the guide sequence comprises a sequence that is complementary to the target sequence. See U.S. Patent Publication No. 2015/0166981, published Jun. 18, 2015, the disclosure of which is incorporated by reference herein. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and may be used with the base editors described herein. Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acids Res. (2013); Briner A E et al., (2014) Guide RNA functional modules direct Cas9 activity and orthogonality, Mol Cell, 56, 333-339, each of which are herein incorporated herein by reference.

(G) Preparation of Base Editors for Increased Expression in Cells

The invention relates in various aspects to methods of making the disclosed base editors by various modes of manipulation that include, but are not limited to, codon optimization of one or more domains of the base editors (e.g., of a guanine oxidase) to achieve greater expression levels in a cell. The base editors contemplated herein can include modifications that result in increased expression through codon optimization and ancestral reconstruction analysis.

In some embodiments, the base editors (or a component thereof) is codon optimized for expression in particular cells, such as eukaryotic cells (e.g., mammalian cells or human cells). The eukaryotic cells may be those of or derived from a particular organism, such as a mammal, including, but not limited to, human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y. et al., “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid. In some embodiments, nucleic acid constructs are codon-optimized for expression in HEK293T cells. In some embodiments, nucleic acid constructs are codon-optimized for expression in mammalian cells. In some embodiments, nucleic acid constructs are codon-optimized for expression in human cells.

In other embodiments, the base editors of the invention have improved expression (as compared to non-modified or state of the art counterpart editors) as a result of ancestral sequence reconstruction analysis. Ancestral sequence reconstruction (ASR) is the process of analyzing modern sequences within an evolutionary/phylogenetic context to infer the ancestral sequences at particular nodes of a tree. Reference is made to Koblan et al., Nat Biotechnol. 2018; 36(9):843-846. These ancient sequences are most often then synthesized, recombinantly expressed in laboratory microorganisms or cell lines, and then characterized to reveal the ancient properties of the extinct biomolecules. This process has produced tremendous insights into the mechanisms of molecular adaptation and functional divergence. Despite such insights, a major criticism of ASR is the general inability to benchmark accuracy of the implemented algorithms. It is difficult to benchmark ASR for many reasons. Notably, genetic material is not preserved in fossils on a long enough time scale to satisfy most ASR studies (many millions to billions of years ago), and it is not yet physically possible to travel back in time to collect samples. Reference can be made to Cal et al., “Reconstruction of ancestral protein sequences and its applications,” BMC Evolutionary Biology 2004, 4:33 and Zakas et al., “Enhancing the pharmaceutical properties of protein drugs by ancestral sequence reconstruction,” Nature Biotechnology, 35-37 (2017), each of which are incorporated herein by reference.

There are many software packages available which can perform ancestral state reconstruction. Generally, these software packages have been developed and maintained through the efforts of scientists in related fields and released under free software licenses. The following list is not meant to be a comprehensive itemization of all available packages, but provides a representative sample of the extensive variety of packages that implement methods of ancestral reconstruction with different strengths and features: PAML (Phylogenetic Analysis by Maximum Likelihood, available at //abacus.gene.ucl.ac.uk/software/paml.html), BEAST (Bayesian evolutionary analysis by sampling trees, available at //www.beast2.org/wiki/index.php/Main_Page), and Diversitree (FitzJohn RG, 2012. Diversitree: comparative phylogenetic analyses of diversification in R. Methods in Ecology and Evolution), and HyPHy (Hypothesis testing using phylogenies, available at //hyphy.org/w/index.php/Main_Page).

The above description is meant to be non-limiting with regard to making base editors having increased expression, and thereby increase editing efficiencies.

(H) Increasing Base Editor Targeting Efficiencies

Some embodiments of the disclosure are based on the recognition that any of the base editors provided herein are capable of modifying a specific nucleobase without generating a significant proportion of indels. An “indel”, as used herein, refers to the insertion or deletion of a nucleobase within a nucleic acid. Such insertions or deletions can lead to frame shift mutations within a coding region of a gene. In some embodiments, it is desirable to generate base editors that efficiently modify (e.g., oxidize or methylate) a specific nucleotide within a nucleic acid, without generating a large number of insertions or deletions (i.e., indels) in the nucleic acid. In certain embodiments, any of the base editors provided herein are capable of generating a greater proportion of intended modifications (e.g., point mutations) versus indels. In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method. In some embodiments, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the base editors provided herein are capable of limiting formation of indels in a region of a nucleic acid. In some embodiments, the region is at a nucleotide targeted by a base editor or a region within 2, 3, 4, 5, 6, 7, 8, 9, 10, 12, 14, 16, 18, 20, or 25 nucleotides of a nucleotide targeted by a base editor. In some embodiments, any of the base editors provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some embodiments of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations. In some embodiments, a intended mutation is a mutation that is generated by a specific base editor bound to a gRNA, specifically designed to generate the intended mutation. In some embodiments, the intended mutation is a mutation associated with a disease, disorder or condition. In some embodiments, the intended mutation is a guanine (G) to thymine (T) point mutation associated with a disease, disorder or condition. In some embodiments, the intended mutation is an adenine (A) to cytosine (C) point mutation associated with a disease, disorder or condition. In some embodiments, the intended mutation is a guanine (G) to thymine (T) point mutation within the coding region of a gene. In some embodiments, the intended mutation is a an adenine (A) to cytosine (C) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that changes a codon to encode a different amino acid. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more.

Some embodiments of the disclosure are based on the recognition that the formation of indels in a region of a nucleic acid may be limited by nicking the non-edited strand opposite to the strand in which edits are introduced. This nick serves to direct mismatch repair machinery to the non-edited strand, ensuring that the chemically modified nucleobase is not interpreted as a lesion by the machinery. This nick may be created by the use of an nCas9. The methods provided in this disclosure comprise cutting (or nicking) the non-edited strand of the double-stranded DNA, for example, wherein the one strand comprises the C of the target G:C nucleobase pair. It should be appreciated that the characteristics of the base editors described in the “Editing DNA or RNA” section, herein, may be applied to any of the fusion proteins, or methods of using the fusion proteins provided herein.

### II. Nucleic Acids, Vectors, Cells, and Methods of Engineering and Producing G-to-T Base-Editors

Some embodiments of this disclosure provide methods of engineering and producing the base editors disclosed herein, or base editor complexes comprising one or more napDNAbp-programming nucleic acid molecules (e.g., Cas9 guide RNAs) and a base editor as provided herein. In addition, some embodiments of the disclosure provide methods of using the base editors for editing a target nucleic acid molecule (e.g., a genomic sequence, an RNA sequence, a cDNA sequence, or a viral DNA sequence).

Vectors and Reagents

Several embodiments of the making and using of the base editors of the invention relate to vector systems comprising one or more vectors, or vectors as such. Vectors may be designed to clone and/or express the base editors as disclosed herein. Vectors may also be designed to clone and/or express one ore more gRNAs having complementarity to the target sequence, as disclosed herein. Vectors may also be designed to transfect the base editors and gRNAs of the disclosure into one or more cells, e.g., a target diseased eukaryotic cell for treatment with the base editor systems and methods disclosed herein.

Vectors can be designed for expression of base editor transcripts (e.g., nucleic acid transcripts, proteins, or enzymes) in prokaryotic or eukaryotic cells. For example, base editor transcripts can be expressed in bacterial cells such as Escherichia coli, insect cells (using baculovirus expression vectors), yeast cells, or mammalian cells. Suitable host cells are discussed further in Goeddel, Gene Expression Technology: Methods In Enzymology 185, Academic Press. San Diego, Calif. (1990). Alternatively, expression vectors encoding one or more base editors described herein can be transcribed and translated in vitro, for example using T7 promoter regulatory sequences and T7 polymerase.

Vectors may be introduced and propagated in a prokaryotic cells. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins.

Fusion expression vectors also may be used to express the base editors of the disclosure. Such vectors generally add a number of amino acids to a protein encoded therein, such as to the amino terminus of the recombinant protein. Such fusion vectors may serve one or more purposes, such as: (i) to increase expression of a recombinant protein; (ii) to increase the solubility of a recombinant protein; and (iii) to aid in the purification of a recombinant protein by acting as a ligand in affinity purification. Often, in fusion expression vectors, a proteolytic cleavage site is introduced at the junction of the fusion domain and the recombinant protein to enable separation of the recombinant protein from the fusion domain subsequent to purification of the fusion protein. Such enzymes, and their cognate recognition sequences, include Factor Xa, thrombin and enterokinase. Exemplary fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods In Enzymology 185, Academic Press, San Diego, Calif. (1990) 60-89).

In some embodiments, a vector is a yeast expression vector for expressing the base editors described herein. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

Directed Evolution Methods (e.g., PACE or PANCE)

Various embodiments of the disclosure relate to providing directed evolution methods and systems (e.g., appropriate vectors, cells, phage, flow vessels, etc.) for engineering of the base editors or base editor domains of the present disclosure.

The directed evolution methods provided herein allow for a gene of interest (e.g., a base editor gene) in a viral vector to be evolved over multiple generations of viral life cycles in a flow of host cells to acquire a desired function or activity.

Some embodiments of this disclosure provide a method of continuous evolution of a gene of interest, comprising (a) contacting a population of host cells with a population of viral vectors comprising the gene of interest, wherein (1) the host cell is amenable to infection by the viral vector; (2) the host cell expresses viral genes required for the generation of viral particles; (3) the expression of at least one viral gene required for the production of an infectious viral particle is dependent on a function of the gene of interest; and (4) the viral vector allows for expression of the protein in the host cell, and can be replicated and packaged into a viral particle by the host cell. In some embodiments, the method comprises (b) contacting the host cells with a mutagen. In some embodiments, the method further comprises (c) incubating the population of host cells under conditions allowing for viral replication and the production of viral particles, wherein host cells are removed from the host cell population, and fresh, uninfected host cells are introduced into the population of host cells, thus replenishing the population of host cells and creating a flow of host cells. The cells are incubated in all embodiments under conditions allowing for the gene of interest to acquire a mutation. In some embodiments, the method further comprises (d) isolating a mutated version of the viral vector, encoding an evolved gene product (e.g., protein), from the population of host cells.

In some embodiments, a method of phage-assisted continuous evolution is provided comprising (a) contacting a population of bacterial host cells with a population of phages that comprise a gene of interest to be evolved and that are deficient in a gene required for the generation of infectious phage, wherein (1) the phage allows for expression of the gene of interest in the host cells; (2) the host cells are suitable host cells for phage infection, replication, and packaging; and (3) the host cells comprise an expression construct encoding the gene required for the generation of infectious phage, wherein expression of the gene is dependent on a function of a gene product of the gene of interest. In some embodiments, the method further comprises (b) incubating the population of host cells under conditions allowing for the mutation of the gene of interest, the production of infectious phage, and the infection of host cells with phage, wherein infected cells are removed from the population of host cells, and wherein the population of host cells is replenished with fresh host cells that have not been infected by the phage. In some embodiments, the method further comprises (c) isolating a mutated phage replication product encoding an evolved protein from the population of host cells.

In some embodiments, the viral vector or the phage is a filamentous phage, for example, an M13 phage, such as an M13 selection phage as described in more detail elsewhere herein. In some such embodiments, the gene required for the production of infectious viral particles is the M13 gene III (gIII).

In some embodiments, the viral vector infects mammalian cells. In some embodiments, the viral vector is a retroviral vector. In some embodiments, the viral vector is a vesicular stomatitis virus (VSV) vector. As a dsRNA virus, VSV has a high mutation rate, and can carry cargo, including a gene of interest, of up to 4.5 kb in length. The generation of infectious VSV particles requires the envelope protein VSV-G, a viral glycoprotein that mediates phosphatidylserine attachment and cell entry. VSV can infect a broad spectrum of host cells, including mammalian and insect cells. VSV is therefore a highly suitable vector for continuous evolution in human, mouse, or insect host cells. Similarly, other retroviral vectors that can be pseudotyped with VSV-G envelope protein are equally suitable for continuous evolution processes as described herein.

It is known to those of skill in the art that many retroviral vectors, for example, Murine Leukemia Virus vectors, or Lentiviral vectors can efficiently be packaged with VSV-G envelope protein as a substitute for the virus's native envelope protein. In some embodiments, such VSV-G packagable vectors are adapted for use in a continuous evolution system in that the native envelope (env) protein (e.g., VSV-G in VSVS vectors, or env in MLV vectors) is deleted from the viral genome, and a gene of interest is inserted into the viral genome under the control of a promoter that is active in the desired host cells. The host cells, in turn, express the VSV-G protein, another env protein suitable for vector pseudotyping, or the viral vector's native env protein, under the control of a promoter the activity of which is dependent on an activity of a product encoded by the gene of interest, so that a viral vector with a mutation leading to T increased activity of the gene of interest will be packaged with higher efficiency than a vector with baseline or a loss-of-function mutation.

In some embodiments, mammalian host cells are subjected to infection by a continuously evolving population of viral vectors, for example, VSV vectors comprising a gene of interest and lacking the VSV-G encoding gene, wherein the host cells comprise a gene encoding the VSV-G protein under the control of a conditional promoter. Such retrovirus-bases system could be a two-vector system (the viral vector and an expression construct comprising a gene encoding the envelope protein), or, alternatively, a helper virus can be employed, for example, a VSV helper virus. A helper virus typically comprises a truncated viral genome deficient of structural elements required to package the genome into viral particles, but including viral genes encoding proteins required for viral genome processing in the host cell, and for the generation of viral particles. In such embodiments, the viral vector-based system could be a three-vector system (the viral vector, the expression construct comprising the envelope protein driven by a conditional promoter, and the helper virus comprising viral functions required for viral genome propagation but not the envelope protein). In some embodiments, expression of the five genes of the VSV genome from a helper virus or expression construct in the host cells, allows for production of infectious viral particles carrying a gene of interest, indicating that unbalanced gene expression permits viral replication at a reduced rate, suggesting that reduced expression of VSV-G would indeed serve as a limiting step in efficient viral production.

One advantage of using a helper virus is that the viral vector can be deficient in genes encoding proteins or other functions provided by the helper virus, and can, accordingly, carry a longer gene of interest. In some embodiments, the helper virus does not express an envelope protein, because expression of a viral envelope protein is known to reduce the infectability of host cells by some viral vectors via receptor interference. Viral vectors, for example retroviral vectors, suitable for continuous evolution processes, their respective envelope proteins, and helper viruses for such vectors, are well known to those of skill in the art. For an overview of some exemplary viral genomes, helper viruses, host cells, and envelope proteins suitable for continuous evolution procedures as described herein, see Coffin et al., Retroviruses, CSHL Press 1997, ISBN0-87969-571-4, incorporated herein.

In some embodiments, the incubating of the host cells is for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles. In certain embodiments, the viral vector is an M13 phage, and the length of a single viral life cycle is about 10-20 minutes.

In some embodiments, a viral vector/host cell combination is chosen in which the life cycle of the viral vector is significantly shorter than the average time between cell divisions of the host cell. Average cell division times and viral vector life cycle times are well known in the art for many cell types and vectors, allowing those of skill in the art to ascertain such host cell/vector combinations. In certain embodiments, host cells are being removed from the population of host cells contacted with the viral vector at a rate that results in the average time of a host cell remaining in the host cell population before being removed to be shorter than the average time between cell divisions of the host cells, but to be longer than the average life cycle of the viral vector employed. The result of this is that the host cells, on average, do not have sufficient time to proliferate during their time in the host cell population while the viral vectors do have sufficient time to infect a host cell, replicate in the host cell, and generate new viral particles during the time a host cell remains in the cell population. This assures that the only replicating nucleic acid in the host cell population is the viral vector, and that the host cell genome, the accessory plasmid, or any other nucleic acid constructs cannot acquire mutations allowing for escape from the selective pressure imposed.

For example, in some embodiments, the average time a host cell remains in the host cell population is about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes.

In some embodiments, the average time a host cell remains in the host cell population depends on how fast the host cells divide and how long infection (or conjugation) requires. In general, the flow rate should be faster than the average time required for cell division, but slow enough to allow viral (or conjugative) propagation. The former will vary, for example, with the media type, and can be delayed by adding cell division inhibitor antibiotics (FtsZ inhibitors in E. coli, etc.). Since the limiting step in continuous evolution is production of the protein required for gene transfer from cell to cell, the flow rate at which the vector washes out will depend on the current activity of the gene(s) of interest. In some embodiments, titratable production of the protein required for the generation of infectious particles, as described herein, can mitigate this problem. In some embodiments, an indicator of phage infection allows computer-controlled optimization of the flow rate for the current activity level in real-time.

In some embodiments, the fresh host cells comprise the accessory plasmid required for selection of viral vectors, for example, the accessory plasmid comprising the gene required for the generation of infectious phage particles that is lacking from the phages being evolved. In some embodiments, the host cells are generated by contacting an uninfected host cell with the relevant vectors, for example, the accessory plasmid and, optionally, a mutagenesis plasmid, and growing an amount of host cells sufficient for the replenishment of the host cell population in a continuous evolution experiment. Methods for the introduction of plasmids and other gene constructs into host cells are well known to those of skill in the art and the invention is not limited in this respect. For bacterial host cells, such methods include, but are not limited to, electroporation and heat-shock of competent cells.

In some embodiments, the accessory plasmid comprises a selection marker, for example, an antibiotic resistance marker, and the fresh host cells are grown in the presence of the respective antibiotic to ensure the presence of the plasmid in the host cells. Where multiple plasmids are present, different markers are typically used. Such selection markers and their use in cell culture are known to those of skill in the art, and the invention is not limited in this respect.

In particular embodiments, a first accessory plasmid comprises gene III, and a second accessory plasmid comprises a T7 RNAP gene deactivated by a G to T mutation, which results in an early stop codon. A third accessory plasmid may comprise a nucleotide encoding a dCas9 fused at the N terminus to the C-terminal half of a fast-splicing intein. An exemplary phage plasmid may comprise a nucleotide encoding a guanine oxidase fused at the C terminus to the N-terminal half of the fast-splicing intein. The full-length base editor is reconstituted from the two intein components.

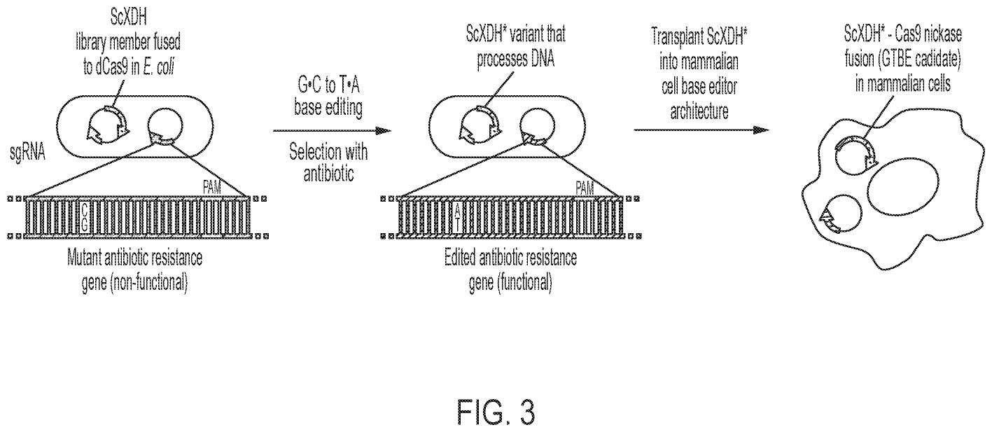

In some embodiments, the selection marker is a spectinomycin antibiotic resistance marker. Cells are transformed with a selection plasmid containing an inactivated spectinomycin resistance gene with a mutation at an active site (K205T) that requires G:C-to-T:A editing to correct. Cells that fail to install the correct transversion mutation in the spectinomycin resistance gene will die, while cells that make the correction will survive. E. coli cells expressing an sgRNA targeting the active site mutation in the spectinomycin resistance gene and a nucleobase modification domain-dCas9 fusion protein are plated onto 2xYT agar with 256 μg/mL of spectinomycin. Surviving colonies (measured through CFUs) were sequenced to find consensus mutations in the fusion proteins expressed in the evolved survivors (FIG. 3). A similar selection assay was used to evolve adenine deaminase activity in DNA during adenine base editor development, as described in Gaudelli, N. M. et al., Programmable base editing of AT to GC in genomic DNA without DNA cleavage. Nature 551, 464-471 (2017), incorporated herein in its entirety by reference.

In some embodiments, the selection marker is a chloramphenicol antibiotic resistance marker. Cells are transformed with a selection plasmid containing an inactivated chloramphenicol resistance gene with a mutation at an active site that requires G:C-to-T:A editing to correct. Cells that fail to install the correct transversion mutation in the chloramphenicol resistance gene will die, while cells that make the correction will survive. E. coli cells expressing an sgRNA targeting the active site mutation in the chloramphenicol resistance gene and a nucleobase modification domain-dCas9 fusion protein are plated onto 2xYT agar with 256 μg/mL of chloramphenicol. Surviving colonies (measured through CFUs) are sequenced to find consensus mutations in the fusion proteins expressed in the evolved survivors.

In other embodiments, the selection marker is a carbenicillin antibiotic resistance marker. Cells are transformed with a selection plasmid containing an inactivated carbenicillin resistance gene with a premature stop codon (Y95X) or a mutation at an active site (S233A or E166A) that requires G:C-to-T:A editing to correct. Cells that fail to install the correct transversion mutation in the carbenicillin resistance gene will die, while cells that make the correction will survive. E. coli cells expressing an sgRNA targeting the active site mutation in the carbenecillin resistance gene and a nucleobase modification domain-dCas9 fusion protein were plated onto 2xYT agar with 256 μg/mL of carbenicillin. Surviving colonies (measured through CFUs) were sequenced to find consensus mutations in the fusion proteins expressed in the evolved survivors.

In some embodiments, the host cell population in a continuous evolution experiment is replenished with fresh host cells growing in a parallel, continuous culture. In some embodiments, the cell density of the host cells in the host cell population contacted with the viral vector and the density of the fresh host cell population is substantially the same.

Typically, the cells being removed from the cell population contacted with the viral vector comprise cells that are infected with the viral vector and uninfected cells. In some embodiments, cells are being removed from the cell populations continuously, for example, by effecting a continuous outflow of the cells from the population. In other embodiments, cells are removed semi-continuously or intermittently from the population. In some embodiments, the replenishment of fresh cells will match the mode of removal of cells from the cell population, for example, if cells are continuously removed, fresh cells will be continuously introduced. However, in some embodiments, the modes of replenishment and removal may be mismatched, for example, a cell population may be continuously replenished with fresh cells, and cells may be removed semi-continuously or in batches.

In some embodiments, the rate of fresh host cell replenishment and/or the rate of host cell removal is adjusted based on quantifying the host cells in the cell population. For example, in some embodiments, the turbidity of culture media comprising the host cell population is monitored and, if the turbidity falls below a threshold level, the ratio of host cell inflow to host cell outflow is adjusted to effect an increase in the number of host cells in the population, as manifested by increased cell culture turbidity. In other embodiments, if the turbidity rises above a threshold level, the ratio of host cell inflow to host cell outflow is adjusted to effect a decrease in the number of host cells in the population, as manifested by decreased cell culture turbidity. Maintaining the density of host cells in the host cell population within a specific density range ensures that enough host cells are available as hosts for the evolving viral vector population, and avoids the depletion of nutrients at the cost of viral packaging and the accumulation of cell-originated toxins from overcrowding the culture.

In some embodiments, the cell density in the host cell population and/or the fresh host cell density in the inflow is about 102 cells/ml to about 1012 cells/ml. In some embodiments, the host cell density is about 102 cells/ml, about 103 cells/ml, about 104 cells/ml, about 105 cells/ml, about 5·105 cells/ml, about 106 cells/ml, about 5·106 cells/ml, about 107 cells/ml, about 5·107 cells/ml, about 108 cells/ml, about 5·108 cells/ml, about 109 cells/ml, about 5·109 cells/ml, about 1010 cells/ml, or about 5·1010 cells/ml. In some embodiments, the host cell density is more than about 1010 cells/ml.

In some embodiments, the host cell population is contacted with a mutagen. In some embodiments, the cell population contacted with the viral vector (e.g., the phage), is continuously exposed to the mutagen at a concentration that allows for an increased mutation rate of the gene of interest, but is not significantly toxic for the host cells during their exposure to the mutagen while in the host cell population. In other embodiments, the host cell population is contacted with the mutagen intermittently, creating phases of increased mutagenesis, and accordingly, of increased viral vector diversification. For example, in some embodiments, the host cells are exposed to a concentration of mutagen sufficient to generate an increased rate of mutagenesis in the gene of interest for about 10%, about 20%, about 50%, or about 75% of the time.

In some embodiments, the host cells comprise a mutagenesis expression construct, for example, in the case of bacterial host cells, a mutagenesis plasmid. In some embodiments, the mutagenesis plasmid comprises a gene expression cassette encoding a mutagenesis-promoting gene product, for example, a proofreading-impaired DNA polymerase. In other embodiments, the mutagenesis plasmid, including a gene involved in the SOS stress response, (e.g., UmuC, UmuD′, and/or RecA). In some embodiments, the mutagenesis-promoting gene is under the control of an inducible promoter. Suitable inducible promoters are well known to those of skill in the art and include, for example, arabinose-inducible promoters, tetracycline or doxycyclin-inducible promoters, and tamoxifen-inducible promoters. In some embodiments, the host cell population is contacted with an inducer of the inducible promoter in an amount sufficient to effect an increased rate of mutagenesis. For example, in some embodiments, a bacterial host cell population is provided in which the host cells comprise a mutagenesis plasmid in which a dnaQ926, UmuC, UmuD′, and RecA expression cassette is controlled by an arabinose-inducible promoter. In some such embodiments, the population of host cells is contacted with the inducer, for example, arabinose in an amount sufficient to induce an increased rate of mutation.

In some embodiments, diversifying the viral vector population is achieved by providing a flow of host cells that does not select for gain-of-function mutations in the gene of interest for replication, mutagenesis, and propagation of the population of viral vectors. In some embodiments, the host cells are host cells that express all genes required for the generation of infectious viral particles, for example, bacterial cells that express a complete helper phage, and, thus, do not impose selective pressure on the gene of interest. In other embodiments, the host cells comprise an accessory plasmid comprising a conditional promoter with a baseline activity sufficient to support viral vector propagation even in the absence of significant gain-of-function mutations of the gene of interest. This can be achieved by using a “leaky” conditional promoter, by using a high-copy number accessory plasmid, thus amplifying baseline leakiness, and/or by using a conditional promoter on which the initial version of the gene of interest effects a low level of activity while a desired gain-of-function mutation effects a significantly higher activity.

Detailed methods of procedures for directing continuous evolution of base editors in a population of host cells using phage particles are disclosed in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, filed Jun. 14, 2019, International Patent Publication WO 2019/023680, published Jan. 31, 2019, International PCT Application, PCT/US2016/027795, filed Apr. 15, 2016, published as WO 2016/168631 on Oct. 20, 2016, and International Patent Publication No. PCT/US2019/47996, filed Aug. 23, 2019, each of which are incorporated herein by reference.

Methods and strategies to design conditional promoters suitable for carrying out the selection strategies described herein are well known to those of skill in the art. For an overview over exemplary suitable selection strategies and methods for designing conditional promoters driving the expression of a gene required for cell-cell gene transfer, e.g., gene III (gIII), see Vidal and Legrain, Yeast n-hybrid review, Nucleic Acids Res. 27, 919 (1999), incorporated herein by reference.

The disclosure provides viral vectors for the continuous evolution processes. In some embodiments, phage vectors for phage-assisted continuous evolution are provided. In some embodiments, a selection phage is provided that comprises a phage genome deficient in at least one gene required for the generation of infectious phage particles and a gene of interest to be evolved.

For example, in some embodiments, the selection phage comprises an M13 phage genome deficient in a gene required for the generation of infectious M13 phage particles, for example, a full-length gIII. In some embodiments, the selection phage comprises a phage genome providing all other phage functions required for the phage life cycle except the gene required for generation of infectious phage particles. In some such embodiments, an M13 selection phage is provided that comprises a gI, gII, gIV, gV, gVI, gVII, gVIII, gIX, and a gX gene, but not a full-length gIII. In some embodiments, the selection phage comprises a 3′-fragment of gIII, but no full-length gIII. The 3′-end of gIII comprises a promoter (see FIG. 16) and retaining this promoter activity is beneficial, in some embodiments, for an increased expression of gVI, which is immediately downstream of the gIII 3′-promoter, or a more balanced (wild-type phage-like) ratio of expression levels of the phage genes in the host cell, which, in turn, can lead to more efficient phage production. In some embodiments, the 3′-fragment of gIII gene comprises the 3′-gIII promoter sequence. In some embodiments, the 3′-fragment of gIII comprises the last 180 bp, the last 150 bp, the last 125 bp, the last 100 bp, the last 50 bp, or the last 25 bp of gIII. In some embodiments, the 3′-fragment of gIII comprises the last 180 bp of gIII.

M13 selection phage is provided that comprises a gene of interest in the phage genome, for example, inserted downstream of the gVIII 3′-terminator and upstream of the gIII-3′-promoter. In some embodiments, an M13 selection phage is provided that comprises a multiple cloning site for cloning a gene of interest into the phage genome, for example, a multiple cloning site (MCS) inserted downstream of the gVIII 3′-terminator and upstream of the gIII-3′-promoter.

Some embodiments of this disclosure provide a vector system for continuous evolution procedures, comprising of a viral vector, for example, a selection phage, and a matching accessory plasmid. In some embodiments, a vector system for phage-based continuous directed evolution is provided that comprises (a) a selection phage comprising a gene of interest to be evolved, wherein the phage genome is deficient in a gene required to generate infectious phage; and (b) an accessory plasmid comprising the gene required to generate infectious phage particle under the control of a conditional promoter, wherein the conditional promoter is activated by a function of a gene product encoded by the gene of interest.

In some embodiments, the selection phage is an M13 phage as described herein. For example, in some embodiments, the selection phage comprises an M13 genome including all genes required for the generation of phage particles, for example, gI, gII, gIV, gV, gVI, gVII, gVIII, gIX, and gX gene, but not a full-length gIII gene. In some embodiments, the selection phage genome comprises an F1 or an M13 origin of replication. In some embodiments, the selection phage genome comprises a 3′-fragment of gIII gene. In some embodiments, the selection phage comprises a multiple cloning site upstream of the gIII 3′-promoter and downstream of the gVIII 3′-terminator.

Some embodiments of this disclosure provide a method of non-continuous evolution of a gene of interest. In certain embodiments, the method of non-continuous evolution is PANCE. In other embodiments, the method of non-continuous evolution is an antibiotic or plate-based selection method.

The PANCE methododology comprises first growing the host strain containing a mutagenesis plasmid of E. coli until optical density reaches A₆₀₀=0.3-0.5 in a large volume. The cells are re-transformed with the mutagenesis plasmid regularly to ensure the plasmid has not been inactivated. An aliquot of a desired concentration, often 2 mL, is then transferred to a smaller flask, supplemented with inducing agent arabinose (Ara) for the mutagenesis plasmid, and infected with the selection phage (SP). To increase the titer level, a drift plasmid can also be provided that enables phage to propagate without passing the selection. Expression is under the control of an inducible promoter and can be turned on with 50 ng/mL of anhydrotetracycline. This culture is incubated at 37° C. for 8-12 h to facilitate phage growth, which is confirmed by determination of the phage titer. Following phage growth, an aliquot of infected cells is used to transfect a subsequent flask containing host E. coli. This process is continued until the desired phenotype is evolved for as many transfers as required, while increasing the stringency in stepwise fashion by decreasing the incubation time or titer of phage with which the bacteria is infected. Reference is made to Suzuki T. et al., Crystal structures reveal an elusive functional domain of pyrrolysyl-tRNA synthetase, Nat Chem Biol. 13(12): 1261-1266 (2017), incorporated herein in its entirety.

In some embodiments, negative selection is applied during a non-continuous evolution method as described herein, by penalizing undesired activities. In some embodiments, this is achieved by causing the undesired activity to interfere with pIII production. For example, expression of an antisense RNA complementary to the gIII RBS and/or start codon is one way of applying negative selection, while expressing a protease (e.g., TEV) and engineering the protease recognition sites into pIII is another.

Other non-continuous selection schemes for gene products having a desired activity are well known to those of skill in the art or will be apparent from the instant disclosure. In certain embodiments, following the successful directed evolution of one or more components of the GTBE base editor (e.g., a Cas9 domain, a guanine oxidase domain, or a guanine methyltransferase domain), methods of making the base editors comprise recombinant protein expression methodologies known to one of ordinary skill in the art.

Editing DNA or RNA

Some embodiments of the disclosure provide methods for editing a nucleic acid using the base editors described herein to effectuate a transversion nucleobase change, e.g., a G:C base pair to a T:A base pair. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to an guanine oxidase) and a guide nucleic acid (e.g., a gRNA), wherein the target region comprises a targeted nucleobase pair, thereby converting a first nucleobase of said target nucleobase pair in a single strand of the target region to a second nucleobase, and optionally cutting (or nicking) no more than one strand of said target region, whereby a third nucleobase complementary to the first nucleobase base is replaced by a fourth nucleobase complementary to the second nucleobase. In certain embodiments, the first nucleobase is a guanine (of the target G:C base pair). In some embodiments, the second nucleobase is a thymine (i.e., the G is converted to T through the intermediate 8-oxo-guanine). In some embodiments, the third nucleobase is also a thymine (of a T:A base pair), and the fourth nucleobase is an adenine. In some embodiments, the second nucleobase is replaced with a fifth nucleobase that is complementary to the fourth nucleobase, thereby generating an intended edited base pair (e.g., G:C pair to a T:A pair).

In some embodiments, the method results in less than 5%, or less than 10%, indel formation in the nucleic acid. In some embodiments, the method results in less than 20% indel formation in the nucleic acid. In other embodiments, the method results in less than 35% indel formation in the nucleic acid. In some embodiments, the first nucleobase is a guanine (of the target G:C base pair). In some embodiments, the second nucleobase is a thymine (e.g., the G is converted to T). In some embodiments, the third nucleobase is also a thymine (of a T:A base pair), and the fourth nucleobase is an adenine. In some embodiments, the method results in less than 19%, 18%, 16%, 14%, 12%, 10%, 8%, 6%, 4%, 2%, 1%, 0.5%, 0.2%, or less than 0.1% indel formation. In some embodiments, at least 5% of the intended base pairs in a population of cells or in tissues in vivo are edited. In some embodiments, at least 10%, 15%, 20%, 25%, 30%, 35%, 40%, 45%, or 50% of the intended base pairs in a population of cells or in tissues in vivo are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand (nicked strand) is hybridized to the guide nucleic acid. In some embodiments, the cut single strand is opposite to the strand comprising the first nucleobase. In some embodiments, the base editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors provided herein. In some embodiments, a target window is a editing window. In some embodiments, the target window is an editing window of 2-20 nucleotides, preferably 2-10 or 2-8 nucleotides.

In another embodiment, the disclosure provides editing methods comprising contacting a DNA, or RNA molecule with any of the base editors provided herein, and with at least one guide nucleic acid (e.g., guide RNA), wherein the guide nucleic acid, (e.g., guide RNA) is about 15-100 nucleotides long and comprises a sequence of at least 10 contiguous nucleotides that is complementary to a target sequence. In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence.

In some embodiments, the target DNA sequence comprises a sequence associated with a disease, disorder or condition. In some embodiments, the complex target nucleic acid sequence comprises a point mutation associated with a disease, disorder, or condition. In some embodiments, the activity of the fusion protein (e.g., comprising a guanine oxidase domain and a napDNAbp domain), or the complex with a gRNA, results in a correction of the point mutation. In some embodiments, the target DNA sequence comprises a T to G point mutation associated with a disease, disorder or condition, and wherein the conversion of the mutant G to a T results in a sequence that is not associated with a disease, disorder, or condition. The target sequence may comprise an A to C point mutation associated with a disease, disorder, or condition, and wherein the conversion of the mutant C to an A results in a sequence that is not associated with a disease, disorder, or condition. In some embodiments, the target nucleic acid sequence encodes a protein, and the point mutation is in a codon and results in a change in the amino acid encoded by the mutant codon as compared to the wild-type codon. In some embodiments, the transversion of the mutant G (or mutant C) results in a change of the amino acid encoded by the mutant codon. In some embodiments, the transversion of the mutant G (or mutant C) results in the codon encoding the wild-type amino acid. In some embodiments, the contacting is in vivo in a subject. In some embodiments, the subject has or has been diagnosed with a disease, disorder or condition. In some embodiments, the disease, disorder or condition is Marfan syndrome or Usher syndrome type 2a.

In some embodiments, the purpose of the methods provided herein is to restore the function of a dysfunctional gene via genome editing. The base editors provided herein can be validated for gene editing-based human therapeutics in vitro, e.g., by correcting a disease-associated mutation in human cell culture. It will be understood by the skilled artisan that the base editors provided herein, e.g., the fusion proteins comprising a nucleic acid programmable DNA binding protein (e.g., Cas9) and a guanine oxidase domain can be used to correct any single point G to T or C to A mutation. Oxidation of the mutant G that is base-paired with the mutant C, followed by a round of replication, corrects the mutation.

The successful correction of point mutations in disease-associated genes and alleles opens up new strategies for gene correction with applications in therapeutics and basic research. Site-specific single-base modification systems like the disclosed fusions of a nucleic acid programmable DNA binding protein and an guanine oxidase domain also have applications in “reverse” gene therapy, where certain gene functions are purposely suppressed or abolished. In these cases, site-specifically mutating residues that lead to inactivating mutations in a protein, or mutations that inhibit function of the protein can be used to abolish or inhibit protein function.

Methods of Treatment

The instant disclosure provides methods for the treatment of a subject diagnosed with a disease associated with or caused by a point mutation that can be corrected by a DNA editing fusion protein provided herein. For example, in some embodiments, a method is provided that comprises administering to a subject having such a disease, e.g., a cancer associated with a point mutation as described above, an effective amount of an guanine oxidase fusion protein and a gRNA that forms a complex with the fusion protein, that corrects the point mutation or introduces a deactivating mutation into a disease-associated gene. In some embodiments, a method is provided that comprises administering to a subject having such a disease, e.g., a cancer associated with a point mutation as described above, an effective amount of an guanine methyltransferase fusion protein-gRNA complex that corrects the point mutation or introduces a deactivating mutation into a disease-associated gene. Further provided herein are methods comprising administering to a subject one or more vectors that contains a nucleotide sequence that expresses the fusion protein and gRNA that forms a complex with the fusion protein.

In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

The instant disclosure provides methods for the treatment of additional diseases or disorders, e.g., diseases or disorders that are associated or caused by a point mutation that can be corrected by guanine oxidase-mediated gene editing. Some such diseases are described herein, and additional suitable diseases that can be treated with the strategies and fusion proteins provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

Exemplary suitable diseases and disorders include, without limitation: 2-methyl-3-hydroxybutyric aciduria; 3 beta-Hydroxysteroid dehydrogenase deficiency; 3-Methylglutaconic aciduria; 3-Oxo-5 alpha-steroid delta 4-dehydrogenase deficiency; 46, XY sex reversal, type 1, 3, and 5; 5-Oxoprolinase deficiency; 6-pyruvoyl-tetrahydropterin synthase deficiency; Aarskog syndrome; Aase syndrome; Achondrogenesis type 2; Achromatopsia 2 and 7; Acquired long QT syndrome; Acrocallosal syndrome, Schinzel type; Acrocapitofemoral dysplasia; Acrodysostosis 2, with or without hormone resistance; Acroerythrokeratoderma; Acromicric dysplasia; Acth-independent macronodular adrenal hyperplasia 2; Activated PI3K-delta syndrome; Acute intermittent porphyria; deficiency of Acyl-CoA dehydrogenase family, member 9; Adams-Oliver syndrome 5 and 6; Adenine phosphoribosyltransferase deficiency; Adenylate kinase deficiency; hemolytic anemia due to Adenylosuccinate lyase deficiency; Adolescent nephronophthisis; Renal-hepatic-pancreatic dysplasia; Meckel syndrome type 7; Adrenoleukodystrophy; Adult junctional epidermolysis bullosa; Epidermolysis bullosa, junctional, localisata variant; Adult neuronal ceroid lipofuscinosis; Adult neuronal ceroid lipofuscinosis; Adult onset ataxia with oculomotor apraxia; ADULT syndrome; Afibrinogenemia and congenital Afibrinogenemia; autosomal recessive Agammaglobulinemia 2; Age-related macular degeneration 3, 6, 11, and 12; Aicardi Goutieres syndromes 1, 4, and 5; Chilbain lupus 1; Alagille syndromes 1 and 2; Alexander disease; Alkaptonuria; Allan-Herndon-Dudley syndrome; Alopecia universalis congenital; Alpers encephalopathy; Alpha-1-antitrypsin deficiency; autosomal dominant, autosomal recessive, and X-linked recessive Alport syndromes; Alzheimer disease, familial, 3, with spastic paraparesis and apraxia; Alzheimer disease, types, 1, 3, and 4; hypocalcification type and hypomaturation type, IIA1 Amelogenesis imperfecta; Aminoacylase 1 deficiency; Amish infantile epilepsy syndrome; Amyloidogenic transthyretin amyloidosis; Amyloid Cardiomyopathy, Transthyretin-related; Cardiomyopathy; Amyotrophic lateral sclerosis types 1, 6, 15 (with or without frontotemporal dementia), 22 (with or without frontotemporal dementia), and 10; Frontotemporal dementia with TDP43 inclusions, TARDBP-related; Andermann syndrome; Andersen Tawil syndrome; Congenital long QT syndrome; Anemia, nonspherocytic hemolytic, due to G6PD deficiency; Angelman syndrome; Severe neonatal-onset encephalopathy with microcephaly; susceptibility to Autism, X-linked 3; Angiopathy, hereditary, with nephropathy, aneurysms, and muscle cramps; Angiotensin i-converting enzyme, benign serum increase; Aniridia, cerebellar ataxia, and mental retardation; Anonychia; Antithrombin III deficiency; Antley-Bixler syndrome with genital anomalies and disordered steroidogenesis; Aortic aneurysm, familial thoracic 4, 6, and 9; Thoracic aortic aneurysms and aortic dissections; Multisystemic smooth muscle dysfunction syndrome; Moyamoya disease 5; Aplastic anemia; Apparent mineralocorticoid excess; Arginase deficiency; Argininosuccinate lyase deficiency; Aromatase deficiency; Arrhythmogenic right ventricular cardiomyopathy types 5, 8, and 10; Primary familial hypertrophic cardiomyopathy; Arthrogryposis multiplex congenita, distal, X-linked; Arthrogryposis renal dysfunction cholestasis syndrome; Arthrogryposis, renal dysfunction, and cholestasis 2; Asparagine synthetase deficiency; Abnormality of neuronal migration; Ataxia with vitamin E deficiency; Ataxia, sensory, autosomal dominant; Ataxia-telangiectasia syndrome; Hereditary cancer-predisposing syndrome; Atransferrinemia; Atrial fibrillation, familial, 11, 12, 13, and 16; Atrial septal defects 2, 4, and 7 (with or without atrioventricular conduction defects); Atrial standstill 2; Atrioventricular septal defect 4; Atrophia bulborum hereditaria; ATR-X syndrome; Auriculocondylar syndrome 2; Autoimmune disease, multisystem, infantile-onset; Autoimmune lymphoproliferative syndrome, type 1a; Autosomal dominant hypohidrotic ectodermal dysplasia; Autosomal dominant progressive external ophthalmoplegia with mitochondrial DNA deletions 1 and 3; Autosomal dominant torsion dystonia 4; Autosomal recessive centronuclear myopathy; Autosomal recessive congenital ichthyosis 1, 2, 3, 4A, and 4B; Autosomal recessive cutis laxa type IA and 1B; Autosomal recessive hypohidrotic ectodermal dysplasia syndrome; Ectodermal dysplasia 11b; hypohidrotic/hair/tooth type, autosomal recessive; Autosomal recessive hypophosphatemic bone disease; Axenfeld-Rieger syndrome type 3; Bainbridge-Ropers syndrome; Bannayan-Riley-Ruvalcaba syndrome; PTEN hamartoma tumor syndrome; Baraitser-Winter syndromes 1 and 2; Barakat syndrome; Bardet-Biedl syndromes 1, 11, 16, and 19; Bare lymphocyte syndrome type 2, complementation group E; Bartter syndrome antenatal type 2; Bartter syndrome types 3, 3 with hypocalciuria, and 4; Basal ganglia calcification, idiopathic, 4; Beaded hair; Benign familial hematuria; Benign familial neonatal seizures 1 and 2; Seizures, benign familial neonatal, 1, and/or myokymia; Seizures, Early infantile epileptic encephalopathy 7; Benign familial neonatal-infantile seizures; Benign hereditary chorea; Benign scapuloperoneal muscular dystrophy with cardiomyopathy; Bernard-Soulier syndrome, types A1 and A2 (autosomal dominant); Bestrophinopathy, autosomal recessive; beta Thalassemia; Bethlem myopathy and Bethlem myopathy 2; Bietti crystalline corneoretinal dystrophy; Bile acid synthesis defect, congenital, 2; Biotinidase deficiency; Birk Barel mental retardation dysmorphism syndrome; Blepharophimosis, ptosis, and epicanthus inversus; Bloom syndrome; Borjeson-Forssman-Lehmann syndrome; Boucher Neuhauser syndrome; Brachydactyly types A1 and A2; Brachydactyly with hypertension; Brain small vessel disease with hemorrhage; Branched-chain ketoacid dehydrogenase kinase deficiency; Branchiootic syndromes 2 and 3; Breast cancer, early-onset; Breast-ovarian cancer, familial 1, 2, and 4; Brittle cornea syndrome 2; Brody myopathy; Bronchiectasis with or without elevated sweat chloride 3; Brown-Vialetto-Van laere syndrome and Brown-Vialetto-Van Laere syndrome 2; Brugada syndrome; Brugada syndrome 1; Ventricular fibrillation; Paroxysmal familial ventricular fibrillation; Brugada syndrome and Brugada syndrome 4; Long QT syndrome; Sudden cardiac death; Bull eye macular dystrophy; Stargardt disease 4; Cone-rod dystrophy 12; Bullous ichthyosiform erythroderma; Burn-Mckeown syndrome; Candidiasis, familial, 2, 5, 6, and 8; Carbohydrate-deficient glycoprotein syndrome type I and II; Carbonic anhydrase VA deficiency, hyperammonemia due to; Carcinoma of colon; Cardiac arrhythmia; Long QT syndrome, LQT1 subtype; Cardioencephalomyopathy, fatal infantile, due to cytochrome c oxidase deficiency; Cardiofaciocutaneous syndrome; Cardiomyopathy; Danon disease; Hypertrophic cardiomyopathy; Left ventricular noncompaction cardiomyopathy; Carnevale syndrome; Carney complex, type 1; Carnitine acylcarnitine translocase deficiency; Carnitine palmitoyltransferase I, II, II (late onset), and II (infantile) deficiency; Cataract 1, 4, autosomal dominant, autosomal dominant, multiple types, with microcornea, coppock-like, juvenile, with microcornea and glucosuria, and nuclear diffuse nonprogressive; Catecholaminergic polymorphic ventricular tachycardia; Caudal regression syndrome; Cd8 deficiency, familial; Central core disease; Centromeric instability of chromosomes 1, 9 and 16 and immunodeficiency; Cerebellar ataxia infantile with progressive external ophthalmoplegi and Cerebellar ataxia, mental retardation, and dysequilibrium syndrome 2; Cerebral amyloid angiopathy, APP-related; Cerebral autosomal dominant and recessive arteriopathy with subcortical infarcts and leukoencephalopathy; Cerebral cavernous malformations 2; Cerebrooculofacioskeletal syndrome 2; Cerebro-oculo-facio-skeletal syndrome; Cerebroretinal microangiopathy with calcifications and cysts; Ceroid lipofuscinosis neuronal 2, 6, 7, and 10; Ch\xc3\xa9diak-Higashi syndrome, Chediak-Higashi syndrome, adult type; Charcot-Marie-Tooth disease types 1B, 2B2, 2C, 2F, 2I, 2U (axonal), 1C (demyelinating), dominant intermediate C, recessive intermediate A, 2A2, 4C, 4D, 4H, IF, IVF, and X; Scapuloperoneal spinal muscular atrophy; Distal spinal muscular atrophy, congenital nonprogressive; Spinal muscular atrophy, distal, autosomal recessive, 5; CHARGE association; Childhood hypophosphatasia; Adult hypophosphatasia; Cholecystitis; Progressive familial intrahepatic cholestasis 3; Cholestasis, intrahepatic, of pregnancy 3; Cholestanol storage disease; Cholesterol monooxygenase (side-chain cleaving) deficiency; Chondrodysplasia Blomstrand type; Chondrodysplasia punctata 1, X-linked recessive and 2 X-linked dominant; CHOPS syndrome; Chronic granulomatous disease, autosomal recessive cytochrome b-positive, types 1 and 2; Chudley-McCullough syndrome; Ciliary dyskinesia, primary, 7, 11, 15, 20 and 22; Citrullinemia type I; Citrullinemia type I and II; Cleidocranial dysostosis; C-like syndrome; Cockayne syndrome type A; Coenzyme Q10 deficiency, primary 1, 4, and 7; Coffin Siris/Intellectual Disability; Coffin-Lowry syndrome; Cohen syndrome; Cold-induced sweating syndrome 1; COLE-CARPENTER SYNDROME 2; Combined cellular and humoral immune defects with granulomas; Combined d-2- and 1-2-hydroxyglutaric aciduria; Combined malonic and methylmalonic aciduria; Combined oxidative phosphorylation deficiencies 1, 3, 4, 12, 15, and 25; Combined partial and complete 17-alpha-hydroxylase/17,20-lyase deficiency; Common variable immunodeficiency 9; Complement component 4, partial deficiency of, due to dysfunctional c1 inhibitor; Complement factor B deficiency; Cone monochromatism; Cone-rod dystrophy 2 and 6; Cone-rod dystrophy amelogenesis imperfecta; Congenital adrenal hyperplasia and Congenital adrenal hypoplasia, X-linked; Congenital amegakaryocytic thrombocytopenia; Congenital aniridia; Congenital central hypoventilation; Hirschsprung disease 3; Congenital contractural arachnodactyly; Congenital contractures of the limbs and face, hypotonia, and developmental delay; Congenital disorder of glycosylation types 1B, 1D, 1G, 1H, 1J, 1K, 1N, 1P, 2C, 2J, 2K, IIm; Congenital dyserythropoietic anemia, type I and II; Congenital ectodermal dysplasia of face; Congenital erythropoietic porphyria; Congenital generalized lipodystrophy type 2; Congenital heart disease, multiple types, 2; Congenital heart disease; Interrupted aortic arch; Congenital lipomatous overgrowth, vascular malformations, and epidermal nevi; Non-small cell lung cancer; Neoplasm of ovary; Cardiac conduction defect, nonspecific; Congenital microvillous atrophy; Congenital muscular dystrophy; Congenital muscular dystrophy due to partial LAMA2 deficiency; Congenital muscular dystrophy-dystroglycanopathy with brain and eye anomalies, types A2, A7, A8, A11, and A14; Congenital muscular dystrophy-dystroglycanopathy with mental retardation, types B2, B3, B5, and B15; Congenital muscular dystrophy-dystroglycanopathy without mental retardation, type B5; Congenital muscular hypertrophy-cerebral syndrome; Congenital myasthenic syndrome, acetazolamide-responsive; Congenital myopathy with fiber type disproportion; Congenital ocular coloboma; Congenital stationary night blindness, type 1A, 1B, 1C, 1E, 1F, and 2A; Coproporphyria; Cornea plana 2; Corneal dystrophy, Fuchs endothelial, 4; Corneal endothelial dystrophy type 2; Corneal fragility keratoglobus, blue sclerae and joint hypermobility; Cornelia de Lange syndromes 1 and 5; Coronary artery disease, autosomal dominant 2; Coronary heart disease; Hyperalphalipoproteinemia 2; Cortical dysplasia, complex, with other brain malformations 5 and 6; Cortical malformations, occipital; Corticosteroid-binding globulin deficiency; Corticosterone methyloxidase type 2 deficiency; Costello syndrome; Cowden syndrome 1; Coxa plana; Craniodiaphyseal dysplasia, autosomal dominant; Craniosynostosis 1 and 4; Craniosynostosis and dental anomalies; Creatine deficiency, X-linked; Crouzon syndrome; Cryptophthalmos syndrome; Cryptorchidism, unilateral or bilateral; Cushing symphalangism; Cutaneous malignant melanoma 1; Cutis laxa with osteodystrophy and with severe pulmonary, gastrointestinal, and urinary abnormalities; Cyanosis, transient neonatal and atypical nephropathic; Cystic fibrosis; Cystinuria; Cytochrome c oxidase i deficiency; Cytochrome-c oxidase deficiency; D-2-hydroxyglutaric aciduria 2; Darier disease, segmental; Deafness with labyrinthine aplasia microtia and microdontia (LAMM); Deafness, autosomal dominant 3a, 4, 12, 13, 15, autosomal dominant nonsyndromic sensorineural 17, 20, and 65; Deafness, autosomal recessive 1A, 2, 3, 6, 8, 9, 12, 15, 16, 18b, 22, 28, 31, 44, 49, 63, 77, 86, and 89; Deafness, cochlear, with myopia and intellectual impairment, without vestibular involvement, autosomal dominant, X-linked 2; Deficiency of 2-methylbutyryl-CoA dehydrogenase; Deficiency of 3-hydroxyacyl-CoA dehydrogenase; Deficiency of alpha-mannosidase; Deficiency of aromatic-L-amino-acid decarboxylase; Deficiency of bisphosphoglycerate mutase; Deficiency of butyryl-CoA dehydrogenase; Deficiency of ferroxidase; Deficiency of galactokinase; Deficiency of guanidinoacetate methyltransferase; Deficiency of hyaluronoglucosaminidase; Deficiency of ribose-5-phosphate isomerase; Deficiency of steroid 11-beta-monooxygenase; Deficiency of UDPglucose-hexose-1-phosphate uridylyltransferase; Deficiency of xanthine oxidase; Dejerine-Sottas disease; Charcot-Marie-Tooth disease, types ID and IVF; Dejerine-Sottas syndrome, autosomal dominant; Dendritic cell, monocyte, B lymphocyte, and natural killer lymphocyte deficiency; Desbuquois dysplasia 2; Desbuquois syndrome; DFNA 2 Nonsyndromic Hearing Loss; Diabetes mellitus and insipidus with optic atrophy and deafness; Diabetes mellitus, type 2, and insulin-dependent, 20; Diamond-Blackfan anemia 1, 5, 8, and 10; Diarrhea 3 (secretory sodium, congenital, syndromic) and 5 (with tufting enteropathy, congenital); Dicarboxylic aminoaciduria; Diffuse palmoplantar keratoderma, Bothnian type; Digitorenocerebral syndrome; Dihydropteridine reductase deficiency; Dilated cardiomyopathy 1A, 1AA, 1C, 1G, 1BB, 1DD, 1FF, 1HH, 1I, 1KK, 1N, 1S, 1Y, and 3B; Left ventricular noncompaction 3; Disordered steroidogenesis due to cytochrome p450 oxidoreductase deficiency; Distal arthrogryposis type 2B; Distal hereditary motor neuronopathy type 2B; Distal myopathy Markesbery-Griggs type; Distal spinal muscular atrophy, X-linked 3; Distichiasis-lymphedema syndrome; Dominant dystrophic epidermolysis bullosa with absence of skin; Dominant hereditary optic atrophy; Donnai Barrow syndrome; Dopamine beta hydroxylase deficiency; Dopamine receptor d2, reduced brain density of; Dowling-degos disease 4; Doyne honeycomb retinal dystrophy; Malattia leventinese; Duane syndrome type 2; Dubin-Johnson syndrome; Duchenne muscular dystrophy; Becker muscular dystrophy; Dysfibrinogenemia; Dyskeratosis congenita autosomal dominant and autosomal dominant, 3; Dyskeratosis congenita, autosomal recessive, 1, 3, 4, and 5; Dyskeratosis congenita X-linked; Dyskinesia, familial, with facial myokymia; Dysplasminogenemia; Dystonia 2 (torsion, autosomal recessive), 3 (torsion, X-linked), 5 (Dopa-responsive type), 10, 12, 16, 25, 26 (Myoclonic); Seizures, benign familial infantile, 2; Early infantile epileptic encephalopathy 2, 4, 7, 9, 10, 11, 13, and 14; Atypical Rett syndrome; Early T cell progenitor acute lymphoblastic leukemia; Ectodermal dysplasia skin fragility syndrome; Ectodermal dysplasia-syndactyly syndrome 1; Ectopia lentis, isolated autosomal recessive and dominant; Ectrodactyly, ectodermal dysplasia, and cleft lip/palate syndrome 3; Ehlers-Danlos syndrome type 7 (autosomal recessive), classic type, type 2 (progeroid), hydroxylysine-deficient, type 4, type 4 variant, and due to tenascin-X deficiency; Eichsfeld type congenital muscular dystrophy; Endocrine-cerebroosteodysplasia; Enhanced s-cone syndrome; Enlarged vestibular aqueduct syndrome; Enterokinase deficiency; Epidermodysplasia verruciformis; Epidermolysa bullosa simplex and limb girdle muscular dystrophy, simplex with mottled pigmentation, simplex with pyloric atresia, simplex, autosomal recessive, and with pyloric atresia; Epidermolytic palmoplantar keratoderma; Familial febrile seizures 8; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Epileptic encephalopathy, childhood-onset, early infantile, 1, 19, 23, 25, 30, and 32; Epiphyseal dysplasia, multiple, with myopia and conductive deafness; Episodic ataxia type 2; Episodic pain syndrome, familial, 3; Epstein syndrome; Fechtner syndrome; Erythropoietic protoporphyria; Estrogen resistance; Exudative vitreoretinopathy 6; Fabry disease and Fabry disease, cardiac variant; Factor H, VII, X, v and factor viii, combined deficiency of 2, xiii, a subunit, deficiency; Familial adenomatous polyposis 1 and 3; Familial amyloid nephropathy with urticaria and deafness; Familial cold urticarial; Familial aplasia of the vermis; Familial benign pemphigus; Familial cancer of breast; Breast cancer, susceptibility to; Osteosarcoma; Pancreatic cancer 3; Familial cardiomyopathy; Familial cold autoinflammatory syndrome 2; Familial colorectal cancer; Familial exudative vitreoretinopathy, X-linked; Familial hemiplegic migraine types 1 and 2; Familial hypercholesterolemia; Familial hypertrophic cardiomyopathy 1, 2, 3, 4, 7, 10, 23 and 24; Familial hypokalemia-hypomagnesemia; Familial hypoplastic, glomerulocystic kidney; Familial infantile myasthenia; Familial juvenile gout; Familial Mediterranean fever and Familial mediterranean fever, autosomal dominant; Familial porencephaly; Familial Porphyria cutanea tarda; Familial pulmonary capillary hemangiomatosis; Familial renal glucosuria; Familial renal hypouricemia; Familial restrictive cardiomyopathy 1; Familial type 1 and 3 hyperlipoproteinemia; Fanconi anemia, complementation group E, I, N, and O; Fanconi-Bickel syndrome; Favism, susceptibility to; Febrile seizures, familial, 11; Feingold syndrome 1; Fetal hemoglobin quantitative trait locus 1; FG syndrome and FG syndrome 4; Fibrosis of extraocular muscles, congenital, 1, 2, 3a (with or without extraocular involvement), 3b; Fish-eye disease; Fleck corneal dystrophy; Floating-Harbor syndrome; Focal epilepsy with speech disorder with or without mental retardation; Focal segmental glomerulosclerosis 5; Forebrain defects; Frank Ter Haar syndrome; Borrone Di Rocco Crovato syndrome; Frasier syndrome; Wilms tumor 1; Freeman-Sheldon syndrome; Frontometaphyseal dysplasia land 3; Frontotemporal dementia; Frontotemporal dementia and/or amyotrophic lateral sclerosis 3 and 4; Frontotemporal Dementia Chromosome 3-Linked and Frontotemporal dementia ubiquitin-positive; Fructose-biphosphatase deficiency; Fuhrmann syndrome; Gamma-aminobutyric acid transaminase deficiency; Gamstorp-Wohlfart syndrome; Gaucher disease type 1 and Subacute neuronopathic; Gaze palsy, familial horizontal, with progressive scoliosis; Generalized dominant dystrophic epidermolysis bullosa; Generalized epilepsy with febrile seizures plus 3, type 1, type 2; Epileptic encephalopathy Lennox-Gastaut type; Giant axonal neuropathy; Glanzmann thrombasthenia; Glaucoma 1, open angle, e, F, and G; Glaucoma 3, primary congenital, d; Glaucoma, congenital and Glaucoma, congenital, Coloboma; Glaucoma, primary open angle, juvenile-onset; Glioma susceptibility 1; Glucose transporter type 1 deficiency syndrome; Glucose-6-phosphate transport defect; GLUT1 deficiency syndrome 2; Epilepsy, idiopathic generalized, susceptibility to, 12; Glutamate formiminotransferase deficiency; Glutaric acidemia IIA and IIB; Glutaric aciduria, type 1; Gluthathione synthetase deficiency; Glycogen storage disease 0 (muscle), II (adult form), IXa2, IXc, type 1A; type II, type IV, IV (combined hepatic and myopathic), type V, and type VI; Goldmann-Favre syndrome; Gordon syndrome; Gorlin syndrome; Holoprosencephaly sequence; Holoprosencephaly 7; Granulomatous disease, chronic, X-linked, variant; Granulosa cell tumor of the ovary; Gray platelet syndrome; Griscelli syndrome type 3; Groenouw corneal dystrophy type I; Growth and mental retardation, mandibulofacial dysostosis, microcephaly, and cleft palate; Growth hormone deficiency with pituitary anomalies; Growth hormone insensitivity with immunodeficiency; GTP cyclohydrolase I deficiency; Hajdu-Cheney syndrome; Hand foot uterus syndrome; Hearing impairment; Hemangioma, capillary infantile; Hematologic neoplasm; Hemochromatosis type 1, 2B, and 3; Microvascular complications of diabetes 7; Transferrin serum level quantitative trait locus 2; Hemoglobin H disease, nondeletional; Hemolytic anemia, nonspherocytic, due to glucose phosphate isomerase deficiency; Hemophagocytic lymphohistiocytosis, familial, 2; Hemophagocytic lymphohistiocytosis, familial, 3; Heparin cofactor II deficiency; Hereditary acrodermatitis enteropathica; Hereditary breast and ovarian cancer syndrome; Ataxia-telangiectasia-like disorder; Hereditary diffuse gastric cancer; Hereditary diffuse leukoencephalopathy with spheroids; Hereditary factors II, IX, VIII deficiency disease; Hereditary hemorrhagic telangiectasia type 2; Hereditary insensitivity to pain with anhidrosis; Hereditary lymphedema type I; Hereditary motor and sensory neuropathy with optic atrophy; Hereditary myopathy with early respiratory failure; Hereditary neuralgic amyotrophy; Hereditary Nonpolyposis Colorectal Neoplasms; Lynch syndrome I and II; Hereditary pancreatitis; Pancreatitis, chronic, susceptibility to; Hereditary sensory and autonomic neuropathy type IIB and IIA; Hereditary sideroblastic anemia; Hermansky-Pudlak syndrome 1, 3, 4, and 6; Heterotaxy, visceral, 2, 4, and 6, autosomal; Heterotaxy, visceral, X-linked; Heterotopia; Histiocytic medullary reticulosis; Histiocytosis-lymphadenopathy plus syndrome; Holocarboxylase synthetase deficiency; Holoprosencephaly 2, 3, 7, and 9; Holt-Oram syndrome; Homocysteinemia due to MTHFR deficiency, CBS deficiency, and Homocystinuria, pyridoxine-responsive; Homocystinuria-Megaloblastic anemia due to defect in cobalamin metabolism, cblE complementation type; Howel-Evans syndrome; Hurler syndrome; Hutchinson-Gilford syndrome; Hydrocephalus; Hyperammonemia, type III; Hypercholesterolaemia and Hypercholesterolemia, autosomal recessive; Hyperekplexia 2 and Hyperekplexia hereditary; Hyperferritinemia cataract syndrome; Hyperglycinuria; Hyperimmunoglobulin D with periodic fever; Mevalonic aciduria; Hyperimmunoglobulin E syndrome; Hyperinsulinemic hypoglycemia familial 3, 4, and 5; Hyperinsulinism-hyperammonemia syndrome; Hyperlysinemia; Hypermanganesemia with dystonia, polycythemia and cirrhosis; Hyperornithinemia-hyperammonemia-homocitrullinuria syndrome; Hyperparathyroidism 1 and 2; Hyperparathyroidism, neonatal severe; Hyperphenylalaninemia, bh4-deficient, a, due to partial pts deficiency, BH4-deficient, D, and non-pku; Hyperphosphatasia with mental retardation syndrome 2, 3, and 4; Hypertrichotic osteochondrodysplasia; Hypobetalipoproteinemia, familial, associated with apob32; Hypocalcemia, autosomal dominant 1; Hypocalciuric hypercalcemia, familial, types 1 and 3; Hypochondrogenesis; Hypochromic microcytic anemia with iron overload; Hypoglycemia with deficiency of glycogen synthetase in the liver; Hypogonadotropic hypogonadism 11 with or without anosmia; Hypohidrotic ectodermal dysplasia with immune deficiency; Hypohidrotic X-linked ectodermal dysplasia; Hypokalemic periodic paralysis 1 and 2; Hypomagnesemia 1, intestinal; Hypomagnesemia, seizures, and mental retardation; Hypomyelinating leukodystrophy 7; Hypoplastic left heart syndrome; Atrioventricular septal defect and common atrioventricular junction; Hypospadias 1 and 2, X-linked; Hypothyroidism, congenital, nongoitrous, 1; Hypotrichosis 8 and 12; Hypotrichosis-lymphedema-telangiectasia syndrome; I blood group system; Ichthyosis bullosa of Siemens; Ichthyosis exfoliativa; Ichthyosis prematurity syndrome; Idiopathic basal ganglia calcification 5; Idiopathic fibrosing alveolitis, chronic form; Dyskeratosis congenita, autosomal dominant, 2 and 5; Idiopathic hypercalcemia of infancy; Immune dysfunction with T-cell inactivation due to calcium entry defect 2; Immunodeficiency 15, 16, 19, 30, 31C, 38, 40, 8, due to defect in cd3-zeta, with hyper IgM type 1 and 2, and X-Linked, with magnesium defect, Epstein-Barr virus infection, and neoplasia; Immunodeficiency-centromeric instability-facial anomalies syndrome 2; Inclusion body myopathy 2 and 3; Nonaka myopathy; Infantile convulsions and paroxysmal choreoathetosis, familial; Infantile cortical hyperostosis; Infantile GM1 gangliosidosis; Infantile hypophosphatasia; Infantile nephronophthisis; Infantile nystagmus, X-linked; Infantile Parkinsonism-dystonia; Infertility associated with multi-tailed spermatozoa and excessive DNA; Insulin resistance; Insulin-resistant diabetes mellitus and acanthosis nigricans; Insulin-dependent diabetes mellitus secretory diarrhea syndrome; Interstitial nephritis, karyomegalic; Intrauterine growth retardation, metaphyseal dysplasia, adrenal hypoplasia congenita, and genital anomalies; lodotyrosyl coupling defect; IRAK4 deficiency; Iridogoniodysgenesis dominant type and type 1; Iron accumulation in brain; Ischiopatellar dysplasia; Islet cell hyperplasia; Isolated 17,20-lyase deficiency; Isolated lutropin deficiency; Isovaleryl-CoA dehydrogenase deficiency; Jankovic Rivera syndrome; Jervell and Lange-Nielsen syndrome 2; Joubert syndrome 1, 6, 7, 9/15 (digenic), 14, 16, and 17, and Orofaciodigital syndrome xiv; Junctional epidermolysis bullosa gravis of Herlitz; Juvenile GM>1<gangliosidosis; Juvenile polyposis syndrome; Juvenile polyposis/hereditary hemorrhagic telangiectasia syndrome; Juvenile retinoschisis; Kabuki make-up syndrome; Kallmann syndrome 1, 2, and 6; Delayed puberty; Kanzaki disease; Karak syndrome; Kartagener syndrome; Kenny-Caffey syndrome type 2; Keppen-Lubinsky syndrome; Keratoconus 1; Keratosis follicularis; Keratosis palmoplantaris striata 1; Kindler syndrome; L-2-hydroxyglutaric aciduria; Larsen syndrome, dominant type; Lattice corneal dystrophy Type III; Leber amaurosis; Zellweger syndrome; Peroxisome biogenesis disorders; Zellweger syndrome spectrum; Leber congenital amaurosis 11, 12, 13, 16, 4, 7, and 9; Leber optic atrophy; Aminoglycoside-induced deafness; Deafness, nonsyndromic sensorineural, mitochondrial; Left ventricular noncompaction 5; Left-right axis malformations; Leigh disease; Mitochondrial short-chain Enoyl-CoA Hydratase 1 deficiency; Leigh syndrome due to mitochondrial complex I deficiency; Leiner disease; Leri Weill dyschondrosteosis; Lethal congenital contracture syndrome 6; Leukocyte adhesion deficiency type I and III; Leukodystrophy, Hypomyelinating, 11 and 6; Leukoencephalopathy with ataxia, with Brainstem and Spinal Cord Involvement and Lactate Elevation, with vanishing white matter, and progressive, with ovarian failure; Leukonychia totalis; Lewy body dementia; Lichtenstein-Knorr Syndrome; Li-Fraumeni syndrome 1; Lig4 syndrome; Limb-girdle muscular dystrophy, type 1B, 2A, 2B, 2D, C1, C5, C9, C14; Congenital muscular dystrophy-dystroglycanopathy with brain and eye anomalies, type A14 and B14; Lipase deficiency combined; Lipid proteinosis; Lipodystrophy, familial partial, type 2 and 3; Lissencephaly 1, 2 (X-linked), 3, 6 (with microcephaly), X-linked; Subcortical laminar heterotopia, X-linked; Liver failure acute infantile; Loeys-Dietz syndrome 1, 2, 3; Long QT syndrome 1, 2, 2/9, 2/5, (digenic), 3, 5 and 5, acquired, susceptibility to; Lung cancer; Lymphedema, hereditary, id; Lymphedema, primary, with myelodysplasia; Lymphoproliferative syndrome 1, 1 (X-linked), and 2; Lysosomal acid lipase deficiency; Macrocephaly, macrosomia, facial dysmorphism syndrome; Macular dystrophy, vitelliform, adult-onset; Malignant hyperthermia susceptibility type 1; Malignant lymphoma, non-Hodgkin; Malignant melanoma; Malignant tumor of prostate; Mandibuloacral dysostosis; Mandibuloacral dysplasia with type A or B lipodystrophy, atypical; Mandibulofacial dysostosis, Treacher Collins type, autosomal recessive; Mannose-binding protein deficiency; Maple syrup urine disease type 1A and type 3; Marden Walker like syndrome; Marfan syndrome; Marinesco-Sj\xc3\xb6gren syndrome; Martsolf syndrome; Maturity-onset diabetes of the young, type 1, type 2, type 11, type 3, and type 9; May-Hegglin anomaly; MYH9 related disorders; Sebastian syndrome; McCune-Albright syndrome; Somatotroph adenoma; Sex cord-stromal tumor; Cushing syndrome; McKusick Kaufman syndrome; McLeod neuroacanthocytosis syndrome; Meckel-Gruber syndrome; Medium-chain acyl-coenzyme A dehydrogenase deficiency; Medulloblastoma; Megalencephalic leukoencephalopathy with subcortical cysts land 2a; Megalencephaly cutis marmorata telangiectatica congenital; PIK3CA Related Overgrowth Spectrum; Megalencephaly-polymicrogyria-polydactyly-hydrocephalus syndrome 2; Megaloblastic anemia, thiamine-responsive, with diabetes mellitus and sensorineural deafness; Meier-Gorlin syndromes land 4; Melnick-Needles syndrome; Meningioma; Mental retardation, X-linked, 3, 21, 30, and 72; Mental retardation and microcephaly with pontine and cerebellar hypoplasia; Mental retardation X-linked syndromic 5; Mental retardation, anterior maxillary protrusion, and strabismus; Mental retardation, autosomal dominant 12, 13, 15, 24, 3, 30, 4, 5, 6, and 9; Mental retardation, autosomal recessive 15, 44, 46, and 5; Mental retardation, stereotypic movements, epilepsy, and/or cerebral malformations; Mental retardation, syndromic, Claes-Jensen type, X-linked; Mental retardation, X-linked, nonspecific, syndromic, Hedera type, and syndromic, wu type; Merosin deficient congenital muscular dystrophy; Metachromatic leukodystrophy juvenile, late infantile, and adult types; Metachromatic leukodystrophy; Metatrophic dysplasia; Methemoglobinemia types I and 2; Methionine adenosyltransferase deficiency, autosomal dominant; Methylmalonic acidemia with homocystinuria; Methylmalonic aciduria cb1B type; Methylmalonic aciduria due to methylmalonyl-CoA mutase deficiency; METHYLMALONIC ACIDURIA, mut(0) TYPE; Microcephalic osteodysplastic primordial dwarfism type 2; Microcephaly with or without chorioretinopathy, lymphedema, or mental retardation; Microcephaly, hiatal hernia and nephrotic syndrome; Microcephaly; Hypoplasia of the corpus callosum; Spastic paraplegia 50, autosomal recessive; Global developmental delay; CNS hypomyelination; Brain atrophy; Microcephaly, normal intelligence and immunodeficiency; Microcephaly-capillary malformation syndrome; Microcytic anemia; Microphthalmia syndromic 5, 7, and 9; Microphthalmia, isolated 3, 5, 6, 8, and with coloboma 6; Microspherophakia; Migraine, familial basilar; Miller syndrome; Minicore myopathy with external ophthalmoplegia; Myopathy, congenital with cores; Mitchell-Riley syndrome; mitochondrial 3-hydroxy-3-methylglutaryl-CoA synthase deficiency; Mitochondrial complex I, II, III, III (nuclear type 2, 4, or 8) deficiency; Mitochondrial DNA depletion syndrome 11, 12 (cardiomyopathic type), 2, 4B (MNGIE type), 8B (MNGIE type); Mitochondrial DNA-depletion syndrome 3 and 7, hepatocerebral types, and 13 (encephalomyopathic type); Mitochondrial phosphate carrier and pyruvate carrier deficiency; Mitochondrial trifunctional protein deficiency; Long-chain 3-hydroxyacyl-CoA dehydrogenase deficiency; Miyoshi muscular dystrophy 1; Myopathy, distal, with anterior tibial onset; Mohr-Tranebjaerg syndrome; Molybdenum cofactor deficiency, complementation group A; Mowat-Wilson syndrome; Mucolipidosis III Gamma; Mucopolysaccharidosis type VI, type VI (severe), and type VII; Mucopolysaccharidosis, MPS-I-H/S, MPS-II, MPS-III-A, MPS-III-B, MPS-III-C, MPS-IV-A, MPS-IV-B; Retinitis Pigmentosa 73; Gangliosidosis GM1 type1 (with cardiac involvement) 3; Multicentric osteolysis nephropathy; Multicentric osteolysis, nodulosis and arthropathy; Multiple congenital anomalies; Atrial septal defect 2; Multiple congenital anomalies-hypotonia-seizures syndrome 3; Multiple Cutaneous and Mucosal Venous Malformations; Multiple endocrine neoplasia, types land 4; Multiple epiphyseal dysplasia 5 or Dominant; Multiple gastrointestinal atresias; Multiple pterygium syndrome Escobar type; Multiple sulfatase deficiency; Multiple synostoses syndrome 3; Muscle AMP guanine oxidase deficiency; Muscle eye brain disease; Muscular dystrophy, congenital, megaconial type; Myasthenia, familial infantile, 1; Myasthenic Syndrome, Congenital, 11, associated with acetylcholine receptor deficiency; Myasthenic Syndrome, Congenital, 17, 2A (slow-channel), 4B (fast-channel), and without tubular aggregates; Myeloperoxidase deficiency; MYH-associated polyposis; Endometrial carcinoma; Myocardial infarction 1; Myoclonic dystonia; Myoclonic-Atonic Epilepsy; Myoclonus with epilepsy with ragged red fibers; Myofibrillar myopathy 1 and ZASP-related; Myoglobinuria, acute recurrent, autosomal recessive; Myoneural gastrointestinal encephalopathy syndrome; Cerebellar ataxia infantile with progressive external ophthalmoplegia; Mitochondrial DNA depletion syndrome 4B, MNGIE type; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Myopia 6; Myosclerosis, autosomal recessive; Myotonia congenital; Congenital myotonia, autosomal dominant and recessive forms; Nail-patella syndrome; Nance-Horan syndrome; Nanophthalmos 2; Navajo neurohepatopathy; Nemaline myopathy 3 and 9; Neonatal hypotonia; Intellectual disability; Seizures; Delayed speech and language development; Mental retardation, autosomal dominant 31; Neonatal intrahepatic cholestasis caused by citrin deficiency; Nephrogenic diabetes insipidus, Nephrogenic diabetes insipidus, X-linked; Nephrolithiasis/osteoporosis, hypophosphatemic, 2; Nephronophthisis 13, 15 and 4; Infertility; Cerebello-oculo-renal syndrome (nephronophthisis, oculomotor apraxia and cerebellar abnormalities); Nephrotic syndrome, type 3, type 5, with or without ocular abnormalities, type 7, and type 9; Nestor-Guillermo progeria syndrome; Neu-Laxova syndrome 1; Neurodegeneration with brain iron accumulation 4 and 6; Neuroferritinopathy; Neurofibromatosis, type land type 2; Neurofibrosarcoma; Neurohypophyseal diabetes insipidus; Neuropathy, Hereditary Sensory, Type IC; Neutral 1 amino acid transport defect; Neutral lipid storage disease with myopathy; Neutrophil immunodeficiency syndrome; Nicolaides-Baraitser syndrome; Niemann-Pick disease type C1, C2, type A, and type C1, adult form; Non-ketotic hyperglycinemia; Noonan syndrome 1 and 4, LEOPARD syndrome 1; Noonan syndrome-like disorder with or without juvenile myelomonocytic leukemia; Normokalemic periodic paralysis, potassium-sensitive; Norum disease; Epilepsy, Hearing Loss, And Mental Retardation Syndrome; Mental Retardation, X-Linked 102 and syndromic 13; Obesity; Ocular albinism, type I; Oculocutaneous albinism type 1B, type 3, and type 4; Oculodentodigital dysplasia; Odontohypophosphatasia; Odontotrichomelic syndrome; Oguchi disease; Oligodontia-colorectal cancer syndrome; Opitz G/BBB syndrome; Optic atrophy 9; Oral-facial-digital syndrome; Ornithine aminotransferase deficiency; Orofacial cleft 11 and 7, Cleft lip/palate-ectodermal dysplasia syndrome; Orstavik Lindemann Solberg syndrome; Osteoarthritis with mild chondrodysplasia; Osteochondritis dissecans; Osteogenesis imperfecta type 12, type 5, type 7, type 8, type I, type III, with normal sclerae, dominant form, recessive perinatal lethal; Osteopathia striata with cranial sclerosis; Osteopetrosis autosomal dominant type 1 and 2, recessive 4, recessive 1, recessive 6; Osteoporosis with pseudoglioma; Oto-palato-digital syndrome, types I and II; Ovarian dysgenesis 1; Ovarioleukodystrophy; Pachyonychia congenita 4 and type 2; Paget disease of bone, familial; Pallister-Hall syndrome; Palmoplantar keratoderma, nonepidermolytic, focal or diffuse; Pancreatic agenesis and congenital heart disease; Papillon-Lef\xc3\xa8vre syndrome; Paragangliomas 3; Paramyotonia congenita of von Eulenburg; Parathyroid carcinoma; Parkinson disease 14, 15, 19 (juvenile-onset), 2, 20 (early-onset), 6, (autosomal recessive early-onset, and 9; Partial albinism; Partial hypoxanthine-guanine phosphoribosyltransferase deficiency; Patterned dystrophy of retinal pigment epithelium; PC-K6a; Pelizaeus-Merzbacher disease; Pendred syndrome; Peripheral demyelinating neuropathy, central dysmyelination; Hirschsprung disease; Permanent neonatal diabetes mellitus; Diabetes mellitus, permanent neonatal, with neurologic features; Neonatal insulin-dependent diabetes mellitus; Maturity-onset diabetes of the young, type 2; Peroxisome biogenesis disorder 14B, 2A, 4A, 5B, 6A, 7A, and 7B; Perrault syndrome 4; Perry syndrome; Persistent hyperinsulinemic hypoglycemia of infancy; familial hyperinsulinism; Phenotypes; Phenylketonuria; Pheochromocytoma; Hereditary Paraganglioma-Pheochromocytoma Syndromes; Paragangliomas 1; Carcinoid tumor of intestine; Cowden syndrome 3; Phosphoglycerate dehydrogenase deficiency; Phosphoglycerate kinase 1 deficiency; Photosensitive trichothiodystrophy; Phytanic acid storage disease; Pick disease; Pierson syndrome; Pigmentary retinal dystrophy; Pigmented nodular adrenocortical disease, primary, 1; Pilomatrixoma; Pitt-Hopkins syndrome; Pituitary dependent hypercortisolism; Pituitary hormone deficiency, combined 1, 2, 3, and 4; Plasminogen activator inhibitor type 1 deficiency; Plasminogen deficiency, type I; Platelet-type bleeding disorder 15 and 8; Poikiloderma, hereditary fibrosing, with tendon contractures, myopathy, and pulmonary fibrosis; Polycystic kidney disease 2, adult type, and infantile type; Polycystic lipomembranous osteodysplasia with sclerosing leukoencephalopathy; Polyglucosan body myopathy 1 with or without immunodeficiency; Polymicrogyria, asymmetric, bilateral frontoparietal; Polyneuropathy, hearing loss, ataxia, retinitis pigmentosa, and cataract; Pontocerebellar hypoplasia type 4; Popliteal pterygium syndrome; Porencephaly 2; Porokeratosis 8, disseminated superficial actinic type; Porphobilinogen synthase deficiency; Porphyria cutanea tarda; Posterior column ataxia with retinitis pigmentosa; Posterior polar cataract type 2; Prader-Willi-like syndrome; Premature ovarian failure 4, 5, 7, and 9; Primary autosomal recessive microcephaly 10, 2, 3, and 5; Primary ciliary dyskinesia 24; Primary dilated cardiomyopathy; Left ventricular noncompaction 6; 4, Left ventricular noncompaction 10; Paroxysmal atrial fibrillation; Primary hyperoxaluria, type I, type, and type III; Primary hypertrophic osteoarthropathy, autosomal recessive 2; Primary hypomagnesemia; Primary open angle glaucoma juvenile onset 1; Primary pulmonary hypertension; Primrose syndrome; Progressive familial heart block type 1B; Progressive familial intrahepatic cholestasis 2 and 3; Progressive intrahepatic cholestasis; Progressive myoclonus epilepsy with ataxia; Progressive pseudorheumatoid dysplasia; Progressive sclerosing poliodystrophy; Prolidase deficiency; Proline dehydrogenase deficiency; Schizophrenia 4; Properdin deficiency, X-linked; Propionic academia; Proprotein convertase 1/3 deficiency; Prostate cancer, hereditary, 2; Protan defect; Proteinuria; Finnish congenital nephrotic syndrome; Proteus syndrome; Breast adenocarcinoma; Pseudoachondroplastic spondyloepiphyseal dysplasia syndrome; Pseudohypoaldosteronism type 1 autosomal dominant and recessive and type 2; Pseudohypoparathyroidism type 1A, Pseudopseudohypoparathyroidism; Pseudoneonatal adrenoleukodystrophy; Pseudoprimary hyperaldosteronism; Pseudoxanthoma elasticum; Generalized arterial calcification of infancy 2; Pseudoxanthoma elasticum-like disorder with multiple coagulation factor deficiency; Psoriasis susceptibility 2; PTEN hamartoma tumor syndrome; Pulmonary arterial hypertension related to hereditary hemorrhagic telangiectasia; Pulmonary Fibrosis And/Or Bone Marrow Failure, Telomere-Related, 1 and 3; Pulmonary hypertension, primary, 1, with hereditary hemorrhagic telangiectasia; Purine-nucleoside phosphorylase deficiency; Pyruvate carboxylase deficiency; Pyruvate dehydrogenase E1-alpha deficiency; Pyruvate kinase deficiency of red cells; Raine syndrome; Rasopathy; Recessive dystrophic epidermolysis bullosa; Nail disorder, nonsyndromic congenital, 8; Reifenstein syndrome; Renal adysplasia; Renal carnitine transport defect; Renal coloboma syndrome; Renal dysplasia; Renal dysplasia, retinal pigmentary dystrophy, cerebellar ataxia and skeletal dysplasia; Renal tubular acidosis, distal, autosomal recessive, with late-onset sensorineural hearing loss, or with hemolytic anemia; Renal tubular acidosis, proximal, with ocular abnormalities and mental retardation; Retinal cone dystrophy 3B; Retinitis pigmentosa; Retinitis pigmentosa 10, 11, 12, 14, 15, 17, and 19; Retinitis pigmentosa 2, 20, 25, 35, 36, 38, 39, 4, 40, 43, 45, 48, 66, 7, 70, 72; Retinoblastoma; Rett disorder; Rhabdoid tumor predisposition syndrome 2; Rhegmatogenous retinal detachment, autosomal dominant; Rhizomelic chondrodysplasia punctata type 2 and type 3; Roberts-SC phocomelia syndrome; Robinow Sorauf syndrome; Robinow syndrome, autosomal recessive, autosomal recessive, with brachy-syn-polydactyly; Rothmund-Thomson syndrome; Rapadilino syndrome; RRM2B-related mitochondrial disease; Rubinstein-Taybi syndrome; Salla disease; Sandhoff disease, adult and infantil types; Sarcoidosis, early-onset; Blau syndrome; Schindler disease, type 1; Schizencephaly; Schizophrenia 15; Schneckenbecken dysplasia; Schwannomatosis 2; Schwartz Jampel syndrome type 1; Sclerocornea, autosomal recessive; Sclerosteosis; Secondary hypothyroidism; Segawa syndrome, autosomal recessive; Senior-Loken syndrome 4 and 5; Sensory ataxic neuropathy, dysarthria, and ophthalmoparesis; Sepiapterin reductase deficiency; SeSAME syndrome; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Severe congenital neutropenia; Severe congenital neutropenia 3, autosomal recessive or dominant; Severe congenital neutropenia and 6, autosomal recessive; Severe myoclonic epilepsy in infancy; Generalized epilepsy with febrile seizures plus, types 1 and 2; Severe X-linked myotubular myopathy; Short QT syndrome 3; Short stature with nonspecific skeletal abnormalities; Short stature, auditory canal atresia, mandibular hypoplasia, skeletal abnormalities; Short stature, onychodysplasia, facial dysmorphism, and hypotrichosis; Primordial dwarfism; Short-rib thoracic dysplasia 11 or 3 with or without polydactyly; Sialidosis type I and II; Silver spastic paraplegia syndrome; Slowed nerve conduction velocity, autosomal dominant; Smith-Lemli-Opitz syndrome; Snyder Robinson syndrome; Somatotroph adenoma; Prolactinoma; familial, Pituitary adenoma predisposition; Sotos syndrome 1 or 2; Spastic ataxia 5, autosomal recessive, Charlevoix-Saguenay type, 1, 10, or 11, autosomal recessive; Amyotrophic lateral sclerosis type 5; Spastic paraplegia 15, 2, 3, 35, 39, 4, autosomal dominant, 55, autosomal recessive, and 5A; Bile acid synthesis defect, congenital, 3; Spermatogenic failure 11, 3, and 8; Spherocytosis types 4 and 5; Spheroid body myopathy; Spinal muscular atrophy, lower extremity predominant 2, autosomal dominant; Spinal muscular atrophy, type II; Spinocerebellar ataxia 14, 21, 35, 40, and 6; Spinocerebellar ataxia autosomal recessive 1 and 16; Splenic hypoplasia; Spondylocarpotarsal synostosis syndrome; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Parastremmatic dwarfism; Stargardt disease 1; Cone-rod dystrophy 3; Stickler syndrome type 1; Kniest dysplasia; Stickler syndrome, types 1 (nonsyndromic ocular) and 4; Sting-associated vasculopathy, infantile-onset; Stormorken syndrome; Sturge-Weber syndrome, Capillary malformations, congenital, 1; Succinyl-CoA acetoacetate transferase deficiency; Sucrase-isomaltase deficiency; Sudden infant death syndrome; Sulfite oxidase deficiency, isolated; Supravalvar aortic stenosis; Surfactant metabolism dysfunction, pulmonary, 2 and 3; Symphalangism, proximal, 1b; Syndactyly Cenani Lenz type; Syndactyly type 3; Syndromic X-linked mental retardation 16; Talipes equinovarus; Tangier disease; TARP syndrome; Tay-Sachs disease, B1 variant, Gm2-gangliosidosis (adult), Gm2-gangliosidosis (adult-onset); Temtamy syndrome; Tenorio Syndrome; Terminal osseous dysplasia; Testosterone 17-beta-dehydrogenase deficiency; Tetraamelia, autosomal recessive; Tetralogy of Fallot; Hypoplastic left heart syndrome 2; Truncus arteriosus; Malformation of the heart and great vessels; Ventricular septal defect 1; Thiel-Behnke corneal dystrophy; Thoracic aortic aneurysms and aortic dissections; Marfanoid habitus; Three M syndrome 2; Thrombocytopenia, platelet dysfunction, hemolysis, and imbalanced globin synthesis; Thrombocytopenia, X-linked; Thrombophilia, hereditary, due to protein C deficiency, autosomal dominant and recessive; Thyroid agenesis; Thyroid cancer, follicular; Thyroid hormone metabolism, abnormal; Thyroid hormone resistance, generalized, autosomal dominant; Thyrotoxic periodic paralysis and Thyrotoxic periodic paralysis 2; Thyrotropin-releasing hormone resistance, generalized; Timothy syndrome; TNF receptor-associated periodic fever syndrome (TRAPS); Tooth agenesis, selective, 3 and 4; Torsades de pointes; Townes-Brocks-branchiootorenal-like syndrome; Transient bullous dermolysis of the newborn; Treacher collins syndrome 1; Trichomegaly with mental retardation, dwarfism and pigmentary degeneration of retina; Trichorhinophalangeal dysplasia type I; Trichorhinophalangeal syndrome type 3; Trimethylaminuria; Tuberous sclerosis syndrome; Lymphangiomyomatosis; Tuberous sclerosis 1 and 2; Tyrosinase-negative oculocutaneous albinism; Tyrosinase-positive oculocutaneous albinism; Tyrosinemia type I; UDPglucose-4-epimerase deficiency; Ullrich congenital muscular dystrophy; Ulna and fibula absence of with severe limb deficiency; Upshaw-Schulman syndrome; Urocanate hydratase deficiency; Usher syndrome, types 1, 1B, 1D, 1G, 2A, 2C, and 2D; Retinitis pigmentosa 39; UV-sensitive syndrome; Van der Woude syndrome; Van Maldergem syndrome 2; Hennekam lymphangiectasia-lymphedema syndrome 2; Variegate porphyria; Ventriculomegaly with cystic kidney disease; Verheij syndrome; Very long chain acyl-CoA dehydrogenase deficiency; Vesicoureteral reflux 8; Visceral heterotaxy 5, autosomal; Visceral myopathy; Vitamin D-dependent rickets, types land 2; Vitelliform dystrophy; von Willebrand disease type 2M and type 3; Waardenburg syndrome type 1, 4C, and 2E (with neurologic involvement); Klein-Waardenberg syndrome; Walker-Warburg congenital muscular dystrophy; Warburg micro syndrome 2 and 4; Warts, hypogammaglobulinemia, infections, and myelokathexis; Weaver syndrome; Weill-Marchesani syndrome 1 and 3; Weill-Marchesani-like syndrome; Weis senbacher-Zweymuller syndrome; Werdnig-Hoffmann disease; Charcot-Marie-Tooth disease; Werner syndrome; WFS1-Related Disorders; Wiedemann-Steiner syndrome; Wilson disease; Wolfram-like syndrome, autosomal dominant; Worth disease; Van Buchem disease type 2; Xeroderma pigmentosum, complementation group b, group D, group E, and group G; X-linked agammaglobulinemia; X-linked hereditary motor and sensory neuropathy; X-linked ichthyosis with steryl-sulfatase deficiency; X-linked periventricular heterotopia; Oto-palato-digital syndrome, type I; X-linked severe combined immunodeficiency; Zimmermann-Laband syndrome and Zimmermann-Laband syndrome 2; and Zonular pulverulent cataract 3.

Pharmaceutical Compositions

Other embodiments of the present disclosure relate to pharmaceutical compositions comprising any of the fusion proteins or the fusion protein-gRNA complexes described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, for targeted delivery, increasing half-life, or other therapeutic compounds).

In some embodiments, any of the fusion proteins, gRNAs, and/or complexes described herein are provided as part of a pharmaceutical composition. In some embodiments, the pharmaceutical composition comprises any of the fusion proteins provided herein. In some embodiments, the pharmaceutical composition comprises any of the complexes provided herein. In some embodiments pharmaceutical composition comprises a gRNA, a napDNAbp-dCas9 fusion protein, and a pharmaceutically acceptable excipient. In some embodiments pharmaceutical composition comprises a gRNA, a napDNAbp-dCas9 fusion protein, and a pharmaceutically acceptable excipient. Pharmaceutical compositions may optionally comprise one or more additional therapeutically active substances.

In some embodiments, compositions provided herein are administered to a subject, for example, to a human subject, in order to effect a targeted genomic modification within the subject. In some embodiments, cells are obtained from the subject and contacted with a any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; 7,163,824; 9,526,784, 9,737,604; and U.S. Patent Publication Nos. 2018/0127780, published May 10, 2018, and 2018/0236081, published Aug. 23, 2018, each of which are incorporated by reference herein. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. See also PCT application PCT/US2010/055131 (Publication No. WO/2011053982), filed Nov. 2, 2010, incorporated herein by reference, for additional suitable methods, reagents, excipients and solvents for producing pharmaceutical compositions comprising a nuclease. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

As used here, the term “pharmaceutically acceptable carrier” means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants may also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; and 9,526,784, each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

Delivery Methods

In some embodiments, the disclosure provides methods comprising delivering any of the fusion proteins, gRNAs, and/or complexes described herein. In other embodiments, the disclosure provides methods comprising delivery of one or more vectors as described herein, one or more transcripts thereof, and/or one or proteins transcribed therefrom, to a host cell. In some embodiments, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include ribonucleoprotein (RNP) complexes, DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds.) (1995); and Yu et al., Gene Therapy 1:13-26 (1994).

In certain embodiments, the method of delivery and vector provided herein is an RNP complex. RNP delivery of base editors markedly increases the DNA specificity of base editing. RNP delivery of base editors leads to decoupling of on- and off-target editing. RNP delivery ablated off-target editing at non-repetitive sites while maintaining on-target editing comparable to plasmid delivery, and greatly reduced off-target editing even at the highly repetitive VEGFA site 2. See Rees, H. A. et al., Improving the DNA specificity and applicability of base editing through protein engineering and protein delivery, Nat. Commun. 8, 15790 (2017), which is incorporated by reference herein in its entirety.

Methods of non-viral delivery of nucleic acids include RNP complexes, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 1991/17424; WO 1991/16024. Delivery can be to cells (e.g., in vitro or ex vivo administration) or target tissues (e.g., in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, 4,946,787, 9,526,784, and 9,737,604).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. Reference is made to US 2003/0087817, published May 8, 2003, International Patent Application No. WO 2016/205764, published Dec. 22, 2016, International Patent Application No. WO2018/071868, published Apr. 19, 2018, U.S. Pat. Nos. 9,526,784, 9,737,604, and U.S. Patent Publication No. 2018/0127780, published May 10, 2018, the disclosures of each of which are incorporated herein by reference.

Kits and Cells

This disclosure provides kits comprising a nucleic acid construct comprising nucleotide sequences encoding the fusion proteins, gRNAs, and/or complexes described herein. Some embodiments of this disclosure provide kits comprising a nucleic acid construct comprising a nucleotide sequence encoding an guanine oxidase-napDNAbp fusion protein capable of recognizing and oxidizing a guanine in a deoxyribonucleic acid (DNA) molecule. Other embodiments of this disclosure provide kits comprising a nucleic acid construct comprising a nucleotide sequence encoding an guanine methyltransferase-napDNAbp fusion protein capable of recognizing and alkylating a guanine in a deoxyribonucleic acid (DNA) molecule. In some embodiments, the nucleotide sequence encodes any of the guanine oxidases provided herein. In some embodiments, the nucleotide sequence comprises a heterologous promoter that drives expression of the fusion protein. The nucleotide sequence may further comprise a heterologous promoter that drives expression of the gRNA, or a heterologous promoter that drives expression of the fusion protein and the gRNA.

In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone.

The disclosure further provides kits comprising a fusion protein as provided herein, a gRNA having complementarity to a target sequence, and one or more of the following: cofactor proteins, buffers, media, and target cells (e.g., human cells). Kits may comprise combinations of several or all of the aforementioned components.

Some embodiments of this disclosure provide kits comprising a nucleic acid construct comprising a nucleotide sequence encoding a guanine methyltransferase-napDNAbp fusion protein capable of alkylating a guanine in a deoxyribonucleic acid (DNA) molecule. In some embodiments, the nucleotide sequence encodes any of the guanine methyltransferases provided herein. In some embodiments, the nucleotide sequence comprises a heterologous promoter that drives expression of the guanine methyltransferase.

Some embodiments of this disclosure provide kits comprising a nucleic acid construct, comprising (a) a nucleotide sequence encoding a napDNAbp (e.g., a Cas9 domain) fused to an guanine oxidase, or a fusion protein comprising a napDNAbp (e.g., Cas9 domain) and an guanine oxidase as provided herein; and (b) a heterologous promoter that drives expression of the sequence of (a). In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone. In some embodiments, the kit further comprises an expression construct comprising a nucleotide sequence encoding an OGG inhibitor.

Some embodiments of this disclosure provide kits comprising a nucleic acid construct, comprising (a) a nucleotide sequence encoding a napDNAbp (e.g., a Cas9 domain) fused to an guanine methyltransferase, or a fusion protein comprising a napDNAbp (e.g., Cas9 domain) and an guanine methyltransferase as provided herein; and (b) a heterologous promoter that drives expression of the sequence of (a). In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone. In some embodiments, the kit further comprises an expression construct comprising a nucleotide sequence encoding an ALRE inhibitor.

Some embodiments of this disclosure provide cells comprising any of the guanine oxidases, guanine methyltransferases, fusion proteins, or complexes provided herein. In some embodiments, the cells comprise a nucleotide that encodes any of the fusion proteins provided herein. In some embodiments, the cells comprise any of the nucleotides or vectors provided herein.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr^{-/-}, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalc1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some aspects, the present disclosure provides uses of any one of the fusion proteins described herein and a guide RNA targeting this fusion protein to a target G:C base pair in a nucleic acid molecule in the manufacture of a kit for nucleic acid editing, wherein the nucleic acid editing comprises contacting the nucleic acid molecule with the fusion protein and guide RNA under conditions suitable for the substitution of the guanine (G) of the G:C nucleobase pair with a thymine. In some embodiments of these uses, the nucleic acid molecule is a double-stranded DNA molecule. In some embodiments, the step of contacting of induces separation of the double-stranded DNA at a target region. In some embodiments, the step of contacting further comprises nicking one strand of the double-stranded DNA, wherein the one strand comprises the T of the target T:A nucleobase pair.

In some embodiments of the described uses, the step of contacting is performed in vitro. In other embodiments, the step of contacting is performed in vivo. In some embodiments, the step of contacting is performed in a subject (e.g., a human subject).

The present disclosure also provides uses of any one of the fusion proteins described herein as a medicament. The present disclosure also provides uses of any one of the complexes of fusion proteins and guide RNAs described herein as a medicament.

### EXAMPLES

### Example 1. Oxidation Approach

Oxidation of guanine to 8-oxo-G induces base rotation, resulting in Hoogsteen pairing of 8-oxo-G with A (FIG. 2A). Streptomyces cyanogenus xanthine dehydrogenase (ScXDH) has been reported to oxidize free guanine to 8-oxo-G without the formation of reactive oxygen species that could damage the cell. ScXDH oxidizes free guanine at C8 with 81% efficiency relative to its native substrate hypoxanthine, and has negligible activity on adenine. Reference is made to Ohe, T. & Watanabe, Y. Purification and Properties of Xanthine Dehydrogenase from Streptomyces cyanogenus, J. Biochem. 86, 45-53 (1979), herein incorporated by reference.

ScXDH was purified and isolated. The ScXDH was tethered to a dCas9 nickase using a SGGSSGGSSGSETPGTSESATPESSGGSSGGS (SEQ ID NO: 11) linker. The fusion protein was introduced to E. coli cells.

Since the protein or gene sequence of ScXDH has not been reported, the protein was submitted for partial sequencing by LC-MS/MS. De novo sequencing of the entire S. cyanogenus genome at 200-fold coverage was completed.

### Example 2. Evolving the ScXDH Base Editor to Recognize a Guanine Target

Using the partial protein sequence from LC-MS/MS and the S. cyanogenus genome sequence, the ScXDH gene was cloned and the activity of the encoded protein confirmed. Variants of ScXDH were evolved using PACE systems to form a large library of ScXDH mutants. Mutants were cloned into a vector coding for an N-terminal fusion with a dCas9. Variants of ScXDH were then evolved using PACE and selected based on ability to convert G into 8-oxo-G in DNA using a carbenicillin antibiotic resistance selection.

Specifically, mutants were subjected to selection based on ability to recognize and oxidize guanine in DNA. The E. coli selection strain was transformed with a) an accessory plasmid containing an ScXDHmutant-dCas9 fusion and targeting guide RNAs, and b) a selection plasmid containing an inactivated carbenicillin resistance gene with a mutation at the active site that requires G:C-to-T:A editing to correct (FIG. 3). Cells harboring ScXDH mutants that restored antibiotic resistance were isolated and subjected to further rounds of mutation and selection under varying selection stringencies.

Because E. coli natively excises 8-oxoguanine with 8-oxo-G glycosylase (OGG), encoded by mutts, selections are performed in the ΔmutM E. coli strain from the Keio collection. Reference is made to Tajiri, T., Maki, H. & Sekiguchi, M., Functional cooperation of MutT, MutM and MutY proteins in preventing mutations caused by spontaneous oxidation of guanine nucleotide in Escherichia coli, Mutat. Res. 336, 257-267 (1995) and Baba, T. et al., Construction of Escherichia coli K-12 in-frame, single-gene knockout mutants: the Keio collection, Mol. Syst. Biol. 2, 2006 0008 (2006), which are incorporated by reference herein.

Those ScXDH variants that conferred a survival advantage to E. coli cells containing the edited selection gene of >100-fold were expressed within a fusion construct comprising a Cas9 nickase, wherein the Cas9 nickase is tethered to the xanthine dehydrogenase variant domain by a linker (e.g., an XTEN linker). The resulting fusion protein was tested for base editing activity in human and murine cells. If 8-oxo-G excision limits editing efficiency, the 8-oxo-G is protected from base excision repair by fusing to the candidate G-to-T base editor (GTBE) to a known catalytically inactivated OGG mutant that retains its ability to tightly bind 8-oxo-G-containing DNA.

Candidate GTBEs were characterized in human (HEK293T) and murine cell lines across ≥30 endogenous genomic loci to assess editing efficiency, product purity, the size of the editing window, and sequence context preferences. Directed evolution is continued until the resulting GTBEs perform at a level useful to the genome editing community (e.g., >20% editing, >80% product purity, <5% indels, and an editing window of 2-8 nucleotides). Similar to studies reported with previous BEs, off-target analysis is performed for candidate GTBEs at Cas9 nuclease off-target sites unrelated to the target site, as identified by GUIDE-seq using the same sgRNAs. See Tsai, S. Q. et al., GUIDE-seq enables genome-wide profiling of off-target cleavage by CRISPR-Cas nucleases. Nature Biotechnology 33, 187-197 (2015), which is incorporated herein.

Successful GTBE development may enable correction of numerous pathogenic mutations, including Marfan syndrome (FBN1 C136G), which affects connective tissue, and Usher syndrome type 2a (USHA2 C934W), which results in hearing and vision loss. See Landrum, M. J. et al., ClinVar: public archive of relationships among sequence variation and human phenotype, Nucleic Acids Res. 42, D980-985 (2014). Candidate GTBEs will be tested on the disease relevant loci in patient-derived cellular models. Based on the results from these studies, ability of the GTBE to prevent vision loss in a previously reported zebra fish model of Usher syndrome type 2a is also tested. See Blanco-Sanchez, B. et al., Zebrafish models of human eye and inner ear diseases, Methods Cell Biol 138, 415-467 (2017).

Other enzymes can be used in this Example, but are not limited to, xanthine dehydrogenase derived from C. capitata, N. crassa, M. hansupus, E. cloacae, S. snoursei, S. albulus, S. himastatinicus, and S. lividans; human CYP1A2, CYP2A6 and CYP3A6; bacterial AlkB; TET1, TET1-CD, TET2 and TET3. Moreover, since XDH enzymes function in E. coli and do not rely on mammalian cell DNA repair processes to mediate G-to-T conversion, the PACE base editor selection system may be used as an alternative evolution platform if stepwise antibiotic selection is unsuccessful.

If ScXDH ultimately proves unsuccessful, selections and evolutions are performed using other candidate oxidizing enzymes that are capable of acting on DNA. These include xanthine dehydrogenase homologs and P450 enzymes, which are known to oxidize purines at C8.

### Example 3. Alkylation Approach

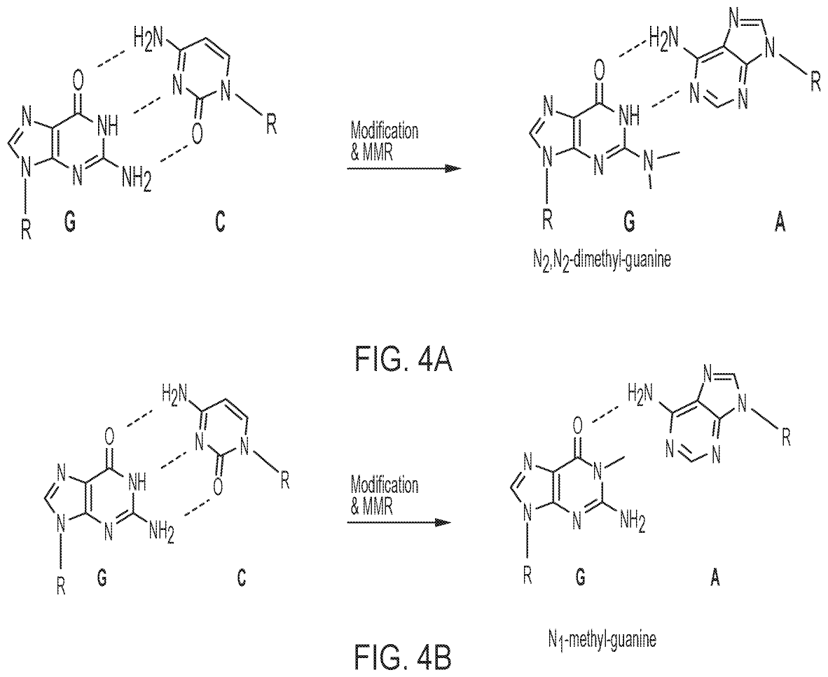

Alkylation of guanine to N₁-methyl guanine, which disrupts existing hydrogen bonding with the cytosine of the unmutated strand. The cell's replication machinery interprets the mutated guanine as a T, and converts the mismatched cytosine to an adenine (FIG. 4). E. coli RlmA has been reported to methylate guanine within RNA to N₁-methyl guanine.

RlmA was purified and isolated. The RlmA was tethered to a dCas9 nickase using a SGGSSGGSSGSETPGTSESATPESSGGSSGGS (SEQ ID NO: 11) linker. The fusion protein was introduced to E. coli cells.

The RlmA protein was submitted for partial sequencing by LC-MS/MS.

### Example 4. Evolving the RlmA Base Editor to Recognize a Guanine Target

The RlmA gene was cloned and the activity of the encoded protein confirmed. Variants of RlmA were then evolved using PACE and PANCE systems and selected based on ability to convert G into N₁-methyl-guanine in DNA using a carbenicillin antibiotic resistance selection.

In another data set, variants were selected based on ability to convert G into N₁-methyl-guanine in DNA using a spectinomycin antibiotic resistance selection. In yet another data set, variants were selected based on ability to convert G into N₁-methyl-guanine in DNA using a chloramphenicol antibiotic resistance selection.

The E. coli selection strain is transformed with an accessory plasmid containing a library of mutagenized RlmA-dCas9 fusions, targeting guide RNAs, and a selection plasmid containing an inactivated carbenicillin resistance gene with a premature stop codon (Y95X) or a mutation at the active site (S233A) that requires G:C-to-T:A editing to correct (FIG. 3). Cells harboring RlmA mutants that restore antibiotic resistance are isolated and subjected to further rounds of mutation and selection under varying selection stringencies.

Those RlmA variants that conferred a survival advantage to E. coli cells containing the edited selection gene of ≥100-fold are tested for base editing activity in human and murine cells. If N₁-methyl-guanine excision limits editing efficiency, the mutated guanine is protected from base excision repair by fusing to the candidate G-to-T base editor (GTBE) to a known catalytically inactivated ALRE that retains its ability to tightly bind N₁-methyl-guanine-containing DNA See, e.g., Norman, D. P., Chung, S. J. & Verdine, G. L., Structural and biochemical exploration of a critical amino acid in human 8-oxoguanine glycosylase, Biochemistry 42, 1564-1572 (2003) and Banerjee, A., Santos, W. L. & Verdine, G. L., Structure of a DNA glycosylase searching for lesions, Science 311, 1153-1157 (2006), each of which are incorporated by reference herein.

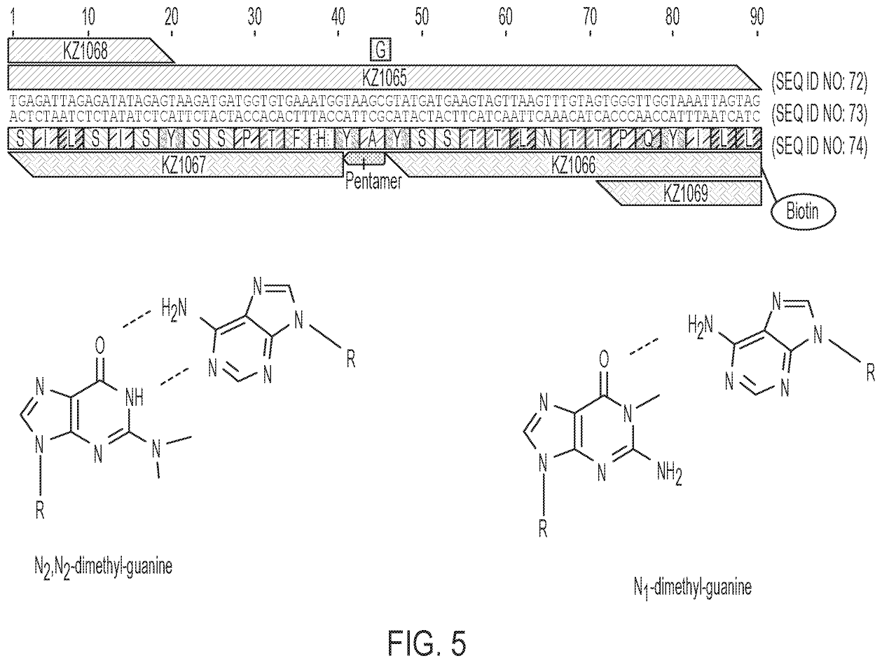

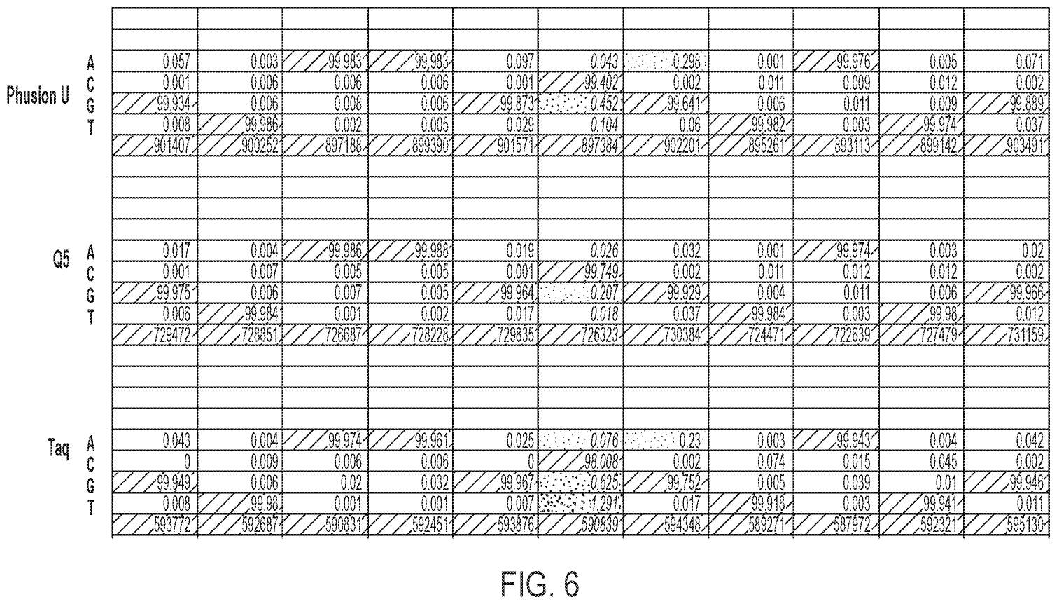

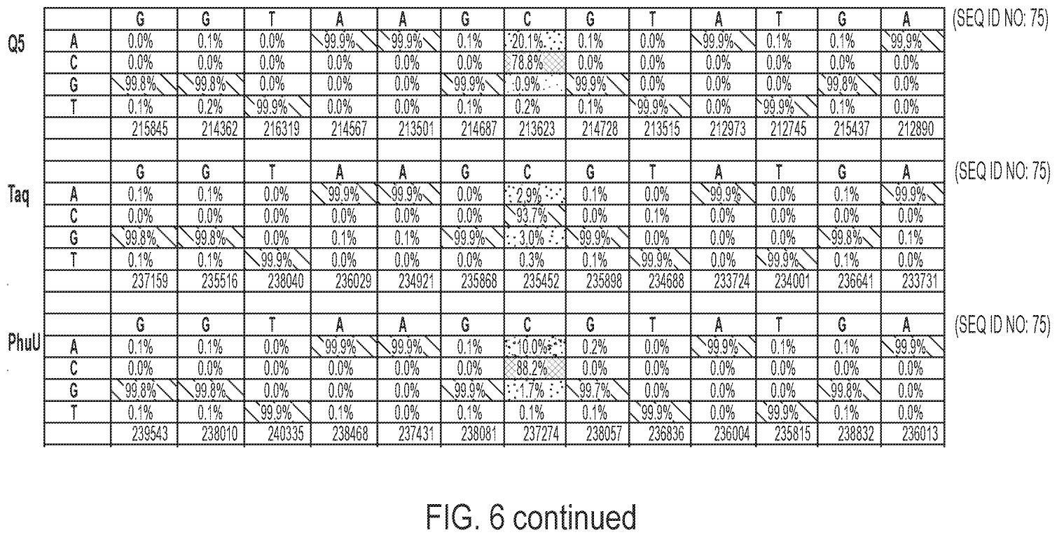

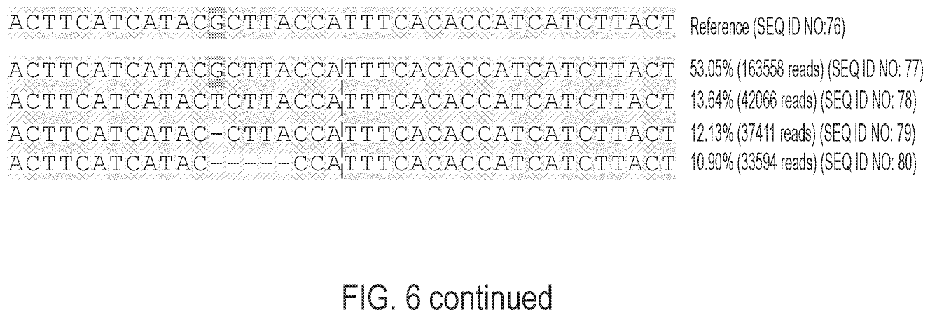

Using phosphoramidite chemistry, 5′-phosphorylated small DNA oligonucleotides containing N₁-methyl-guanine were synthesized using standard automated oligonucleotide synthesis with commercially available amine-modified nucleoside phosphoramidites and 5′-phosphorylation reagents. See Hili R. et al., DNA Ligase-Mediated Translation of DNA Into Densely Functionalized Nucleic Acid Polymers, J. Am. Chem. Soc. 135(1): 98-101 (2013). These functionalized oligonucleotides were purified by reverse-phase HPLC and subsequently incorporated into a larger fragment through in vitro ligation with biotin ligase tags. After transformation of the fragment into mammalian cells, a biotin pull-down was performed to purify a single strand (FIG. 5). Bacterial (non-mammalian) polymerases were applied to the pulled-down strand to identify the potential mutagenic effect. Bacterial polymerases used in this Example include Phusion U® (Thermo Scientific), Q5® (NEB), and Taq polymerases (FIG. 6).

If Rlma ultimately proves unsuccessful, selections and evolutions are performed using other candidate N₁-methyl-guanine generating enzymes that are known to methylate purines at N₁. These enzymes include, but are not limited to, Aquifex aeolicus Trm1, human Trm1, Saccharomyces cerevisiae Trm1, human TrmT10A, E. coli TrmD, M. jannaschii Trm5b, P. abyssi Trm5a and the Trm5c of a suitable archaeon.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or embodiments of the invention, is/are referred to as comprising particular elements and/or features, certain embodiments of the invention or embodiments of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A fusion protein comprising: (i) a nucleic acid programmable DNA binding protein (napDNAbp), and (ii) a guanine oxidase.

2. The fusion protein of claim 1, wherein the guanine oxidase oxidizes guanine to 8-oxoguanine (8-oxo-G).

3. The fusion protein of claim 1 or 2, wherein the guanine oxidase oxidizes a guanine in deoxyribonucleic acid (DNA).

4. The fusion protein of any one of claims 1-3, wherein the guanine oxidase is a wild-type guanine oxidase, or a variant thereof, that oxidizes a guanine in DNA.

5. The fusion protein of any one of claims 1-4, wherein the guanine oxidase is a xanthine dehydrogenase, or a variant thereof, that oxidizes a guanine in DNA.

6. The fusion protein of any one of claims 1-5, wherein the guanine oxidase is a Streptomyces cyanogenus xanthine dehydrogenase (ScXDH), or a variant thereof, that oxidizes a guanine in DNA.

7. The fusion protein of any one of claims 1-4, wherein the guanine oxidase is a P450 enzyme, or a variant thereof, that oxidizes a guanine in DNA.

8. The fusion protein of any one of claims 1-4, wherein the guanine oxidase is a TET-oxidase, or a variant thereof, that oxidizes a guanine in DNA.

9. The fusion protein of any one of claims 1-4, wherein the guanine oxidase is an AlkB, or a variant thereof, that oxidizes a guanine in DNA.

10. The fusion protein of any one of claims 1-7, wherein the guanine oxidase comprises an amino acid sequence that is at least 80%, 85%, 90%, 95%, 98%, or 99% identical to the amino acid sequence of any one of SEQ ID NOs: 5-8, SEQ ID NO: 10, SEQ ID NOs: 15-20, SEQ ID NOs: 35-41, or SEQ ID NO: 43.

11. The fusion protein of any one of claims 1-10, wherein the guanine oxidase comprises any one of the amino acid sequences of SEQ ID NO: 5, SEQ ID NO: 19, SEQ ID NO: 20, SEQ ID NO: 35, SEQ ID NO: 36, SEQ ID NO: 38, SEQ ID NO: 39, SEQ ID NO: 40, or SEQ ID NO: 41.

12. The fusion protein of any one of claims 4-11, wherein the variant of the wild-type guanine oxidase is produced by evolving an oxidase enzyme.

13. The fusion protein of claim 12, wherein the step of evolving comprises phage assisted continuous evolution (PACE).

14. The fusion protein of any one of claims 1-13, wherein the nucleic acid programmable DNA binding protein (napDNAbp) is a Cas9 domain, a Cpf1, a CasX, a CasY, a C2c1, a C2c2, a C2c3, a GeoCas9, a CjCas9, a Cas12a, a Cas14 or an Argonaute protein.

15. The fusion protein of claim 14, wherein the Cas9 domain is a nuclease dead Cas9 (dCas9), a Cas9 nickase (nCas9), or a nuclease active Cas9.

16. The fusion protein of any one of claims 1-15, further comprising: (iii) an 8-oxoguanine glycosylase (OGG) inhibitor.

17. The fusion protein of claim 16, wherein the OGG inhibitor binds to 8-oxoguanine (8-oxo-G).

18. The fusion protein of claim 17, wherein the OGG inhibitor comprises a catalytically inactive OGG that binds 8-oxoguanine (8-oxo-G).

19. The fusion protein of any one of claims 1-18, wherein the fusion protein comprises the structure NH₂-[napDNAbp]-[guanine oxidase]-COOH; or NH₂-[guanine oxidase]-[napDNAbp]-COOH, wherein each instance of “]-[” indicates the presence of an optional linker sequence.

20. The fusion protein of claim 19, wherein the napDNAbp and the guanine oxidase are fused via a linker comprising the amino acid sequence
(SEQ ID NO: 11)
SGGSSGGSSGSETPGTSEATPESSGGSSGGS,
(SEQ ID NO: 1)
GGG, GGGS,
(SEQ ID NO: 2)
SGGGS,
or
(SEQ ID NO: 99)
SGSETPGTSESATPES.

21. The fusion protein of any one of claims 16-20, wherein the fusion protein comprises the structure
NH₂-[OGG inhibitor]-[napDNAbp]-[guanine oxidase]-COOH;
NH₂-[napDNAbp]-[OGG inhibitor]-[guanine oxidase]-COOH;
NH₂-[napDNAbp]-[guanine oxidase]-[OGG inhibitor]-COOH;
NH₂-[OGG inhibitor]-[guanine oxidase]-[napDNAbp]-COOH;
NH₂-[guanine oxidase]-[OGG inhibitor][napDNAbp]-COOH; or
NH₂-[guanine oxidase]-[napDNAbp]-[OGG inhibitor]-COOH, wherein each instance of “]-[” indicates the presence of an optional linker sequence.

22. The fusion protein of claim 21, wherein the napDNAbp and the guanine oxidase are fused via a linker comprising the amino acid sequence
(SEQ ID NO: 11)
SGGSSGGSSGSETPGTSEATPESSGGSSGGS,
(SEQ ID NO: 1)
GGG, GGGS,
(SEQ ID NO: 2)
SGGGS,
or
(SEQ ID NO: 99)
SGSETPGTSESATPES.

23. The fusion protein of claim 22, wherein the napDNAbp and the OGG inhibitor are fused via a linker comprising the amino acid sequence
(SEQ ID NO: 11)
SGGSSGGSSGSETPGTSEATPESSGGSSGGS,
(SEQ ID NO: 1)
GGG, GGGS,
(SEQ ID NO: 2)
SGGGS,
or
(SEQ ID NO: 99)
SGSETPGTSESATPES.

24. The fusion protein of claim 21, wherein the guanine oxidase and the OGG inhibitor are fused via a linker comprising the amino acid sequence
(SEQ ID NO: 11)
SGGSSGGSSGSETPGTSEATPESSGGSSGGS,
(SEQ ID NO: 1)
GGG, GGGS,
(SEQ ID NO: 2)
SGGGS,
or
(SEQ ID NO: 99)
SGSETPGTSESATPES.

25. A fusion protein comprising: (i) a nucleic acid programmable DNA binding protein (napDNAbp), and (ii) a guanine methyltransferase.

26. The fusion protein of claim 25, wherein the guanine methyltransferase methylates a guanine to 8-methyl-guanine.

27. The fusion protein of claim 25 or 26, wherein the guanine methyltransferase is a Cfr, or a variant thereof, that methylates a guanine in DNA.

28. The fusion protein of claim 27, wherein the Cfr is a Staphylococcus scirui Cfr, or a variant thereof, that methylates a guanine in DNA.

29. The fusion protein of claim 25, wherein the guanine methyltransferase is a dimethyltransferase that methylates a guanine to N₂,N₂-dimethylguanine.

30. The fusion protein of claim 29, wherein the dimethyltransferase is a Trm1, or a variant thereof, that methylates a guanine in DNA.

31. The fusion protein of claim 30, wherein the dimethyltransferase is a Aquifex aeolicus Trm1, or a variant thereof, that methylates a guanine in DNA.

32. The fusion protein of claim 30, wherein the dimethyltransferase is a Homo sapiens Trm1, or a variant thereof, that methylates a guanine in DNA.

33. The fusion protein of claim 30, wherein the dimethyltransferase is a Saccharomyces cerevisiae Trm1, or a variant thereof, that methylates a guanine in DNA.

34. The fusion protein of claim 25, wherein the guanine methyltransferase methylates a guanine to N₁-methyl-guanine.

35. The fusion protein of claim 34, wherein the methyltransferase is a RlmA, a TrmT10A, a TrmD, Trm5a, Trm5b, Trm5c, or a variant thereof, that methylates a guanine in DNA.

36. The fusion protein of claim 34 or 35, wherein the methyltransferase is an Escherichia coli RlmA, or a variant thereof, that methylates a guanine in DNA.

37. The fusion protein of claim 34 or 35, wherein the methyltransferase is a Homo sapiens TrmT10A, or a variant thereof, that methylates a guanine in DNA.

38. The fusion protein of claim 34 or 35, wherein the methyltransferase is an Escherichia coli TrmD, or a variant thereof, that methylates a guanine in DNA.

39. The fusion protein of claim 34 or 35, wherein the methyltransferase is a Methanocaldococcus jannaschii Trm5b, or a variant thereof, that methylates a guanine in DNA.

40. The fusion protein of claim 34 or 35, wherein the methyltransferase is a Pyrococcus Abyssi Trm5a, or a variant thereof, that methylates a guanine DNA.

41. The fusion protein of any one of claims 25-40, wherein the guanine methyltransferase methylates a guanine in deoxyribonucleic acid (DNA).

42. The fusion protein of any one of claims 25-41, wherein the guanine methyltransferase is a wild-type guanine methyltransferase, or a variant thereof, that methylates a guanine in DNA.

43. The fusion protein of any one of claims 25-42, wherein the guanine methyltransferase comprises an amino acid sequence that is at least 80%, 85%, 90%, 95%, 98%, or 99% identical to the amino acid sequence of any one of SEQ ID NO: 44 or SEQ ID NOs: 46-53.

44. The fusion protein of any one of claims 25-43, wherein the guanine methyltransferase comprises any one of the amino acid sequences of SEQ ID NO: 44, SEQ ID NO: 49, SEQ ID NO: 50, or SEQ ID NO: 51.

45. The fusion protein of any one of claims 27-44, wherein the variant of the wild-type guanine methyltransferase is produced by evolving a methyltransferase enzyme.

46. The fusion protein of any one of claim 45, wherein the evolving includes phage assisted continuous evolution (PACE).

47. The fusion protein of any one of claims 25-46, wherein the nucleic acid programmable DNA binding protein (napDNAbp) is a Cas9 domain, a Cpf1, a CasX, a CasY, a C2c1, a C2c2, a C2c3, a GeoCas9, a CjCas9, a Cas12a, a Cas14 or an Argonaute protein.

48. The fusion protein of claim 47, wherein the Cas9 domain is a nuclease dead Cas9 (dCas9), a Cas9 nickase (nCas9), or a nuclease active Cas9.

49. The fusion protein of any one of claims 25-48, wherein the fusion protein comprises the structure NH₂-[napDNAbp]-[guanine methyltransferase]-COOH; or NH₂-[guanine methyltransferase]-[napDNAbp]-COOH, wherein each instance of “]-[” indicates the presence of an optional linker sequence.

50. The fusion protein of claim 49, wherein the napDNAbp and the guanine methyltransferase are fused via a linker comprising the amino acid sequence
(SEQ ID NO: 11)
SGGSSGGSSGSETPGTSEATPESSGGSSGGS,
(SEQ ID NO: 1)
GGG, GGGS,
(SEQ ID NO: 2)
SGGGS,
or
(SEQ ID NO: 99)
SGSETPGTSESATPES.

51. A polynucleotide encoding the fusion protein of any one of claims 1-50.

52. A vector comprising the polynucleotide of claim 51.

53. The vector of claim 52, wherein the vector comprises a heterologous promoter driving expression of the polynucleotide.

54. A complex comprising the fusion protein of any one of claims 1-50 and a guide RNA bound to the nucleic acid programmable DNA binding protein (napDNAbp) of the fusion protein.

55. A cell comprising the fusion protein of any one of claims 1-50 the polynucleotide of claim 51, the vector of claim 52 or 53, or the complex of claim 54.

56. A pharmaceutical composition comprising:
(i) the fusion protein of any one of claims 1-50, the polynucleotide of claim 51, the vector of claim 52 or 53, or the complex of claim 54; and
(ii) a pharmaceutically acceptable excipient.

57. A kit comprising a nucleic acid construct, comprising
(i) a nucleic acid sequence encoding the fusion protein of any one of claims 1-50; and
(ii) a heterologous promoter that drives expression of the sequence of (a).

58. The kit of claim 57, further comprising an expression construct encoding a guide RNA backbone, wherein the construct comprises a cloning site positioned to allow the cloning of a nucleic acid sequence identical or complementary to a target sequence into the guide RNA backbone.

59. A method for editing a nucleobase pair of a double-stranded DNA sequence, the method comprising:
(i) contacting a double-stranded DNA sequence with a complex comprising a nucleobase editor and a guide nucleic acid, wherein the double-stranded DNA comprises a target G:C nucleobase pair; and
(ii) oxidizing the guanine (G) of the G:C nucleobase pair to 8-oxoguanine (8-oxo-G).

60. A method for editing a nucleobase pair of a double-stranded DNA sequence, the method comprising:
(i) contacting a double-stranded DNA sequence with a complex comprising a nucleobase editor and a guide nucleic acid, wherein the double-stranded DNA comprises a target G:C nucleobase pair; and
(ii) methylating the guanine (G) of the G:C nucleobase pair to N₂,N₂-dimethyl-guanine.

61. A method for editing a nucleobase pair of a double-stranded DNA sequence, the method comprising:
(i) contacting a double-stranded DNA sequence with a complex comprising a nucleobase editor and a guide nucleic acid, wherein the double-stranded DNA comprises a target G:C nucleobase pair; and
(ii) methylating the guanine (G) of the G:C nucleobase pair to N₁-methyl-guanine.

62. The method of any of claims 59-61, wherein the nucleobase editor is the fusion protein of any one of claims 1-50.

63. The method of claim any of claims 59-62, wherein the contacting of (i) induces separation of the double-stranded DNA at a target region.

64. The method of any one of claims 59-63, further comprising:
(iii) cutting one strand of the double-stranded DNA, wherein the one strand comprises the C of the target G:C nucleobase pair.

65. The method of any one of claims 59-64, wherein the C of the target G:C nucleobase pair is replaced with an adenine.

66. The method of any one of claims 59-65, wherein the 8-oxo-G, the N₂,N₂-dimethyl-guanine, or the N₁-methyl-guanine is replaced with a thymine T, thereby generating a G to T point mutation.

67. A method comprising:
(i) contacting a double-stranded DNA sequence with a complex comprising the fusion protein of any one of claims 1-50 and a guide nucleic acid, wherein the double-stranded DNA comprises a target G:C nucleobase pair;
(ii) oxidizing the guanine (G) of the G:C nucleobase pair to 8-oxoguanine (8-oxo-G); and
(iii) cutting one strand of the double-stranded DNA, wherein the one strand comprises the C of the target G:C nucleobase pair, and wherein the C of the target G:C nucleobase pair is replaced with an adenine.

68. A method comprising:
(i) contacting a double-stranded DNA sequence with a complex comprising the fusion protein of any one of claims 1-50 and a guide nucleic acid, wherein the double-stranded DNA comprises a target G:C nucleobase pair;
(ii) methylating the guanine (G) of the G:C nucleobase pair to N₂,N₂-dimethyl-guanine; and
(iii) cutting one strand of the double-stranded DNA, wherein the one strand comprises the C of the target G:C nucleobase pair, and wherein the C of the target G:C nucleobase pair is replaced with an adenine.

69. A method comprising:
(i) contacting a double-stranded DNA sequence with a complex comprising the fusion protein of any one of claims 1-50 and a guide nucleic acid, wherein the double-stranded DNA comprises a target G:C nucleobase pair;
(ii) methylating the guanine (G) of the G:C nucleobase pair to N₁-methyl-guanine; and
(iii) cutting one strand of the double-stranded DNA, wherein the one strand comprises the C of the target G:C nucleobase pair, and wherein the C of the target G:C nucleobase pair is replaced with an adenine.

70. The method of any of claims 67-69, wherein the 8-oxo-G, the N₂,N₂-dimethyl-guanine, or the N₁-methyl-guanine is replaced with a thymine T, thereby generating a G to T point mutation.

71. The method of any one of claims 59-70, wherein the method is performed in vitro, in vivo, or ex vivo.

72. The method of any one of claims 59-71, wherein the double-stranded DNA is in a subject.

73. The method of claim 72, wherein the subject is human.

74. A method of treating a subject having or at risk of developing a disease, disorder or condition, the method comprising:
administering to the subject the fusion protein the fusion protein of any one of claims 1-50, the polynucleotide of claim 51, the vector of claim 52 or 53, the complex of claim 54, or the pharmaceutical composition of claim 56.

75. The method of claim 74, wherein the subject has been diagnosed with a disease, disorder or condition.

76. The method of claim 74 or 75, wherein the subject has a G to T or a C to A mutation that is associated with a disease, disorder or condition.

77. The method of claim 76, wherein the T of the G to T mutation is converted to a G.

78. The method of claim 76 or 77, wherein the A of the C to A mutation is converted to a C.


SEQUENCE LISTING (100 sequences)

SEQ ID NO: 1 - protein, 4 aa, Artificial Sequence
  Synthetic polypeptide
GGGS

SEQ ID NO: 2 - protein, 5 aa, Artificial Sequence
  Synthetic polypeptide
SGGGS

SEQ ID NO: 3 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 4 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 5 - protein, 806 aa, S. cyanogenus
MSHLSERPEKPVVGVSMPHESAVQHVTGAALYTDDLVQRTKDVLHAYPVQVMKARGRVTALRTGAALAVPGVVRVLTGADVPGVNDAGMKHDEPLFPDEVMFHGHAVAWVLGETLEAARIGAAAVEVDLEELPSVITLQDAIAADSYHGARPVMTHGDVDAGFADSAHVFTGEFQFSGQEHFYLETHAALAQVDENGQVFIQSSTQHPSETQEIVSHVLGVPAHEVTVQCLRMGGGFGGKEMQPHGFAAIAALGAKLTGRPVRFRLNRTQDLTMSGKRHGFHATWKIGFDTEGRIQALDATLTADGGWSLDLSEPVLARALCHIDNTYWIPNARVAGRIARTNTVSNTAFRGFGGPQGMLVIEDILGRCAPRLGVDAKELRERNFYRPGQGQTTPYGQPVTQPERIAAVWQQVQDNGHIADREREIAAFNAAHPHTKRALAVTGVKFGISFNLTAFNQGGALVLIYKDGSVLINHGGTEMGQGLHTKMLQVAATTLGIPLHKVRLAPTRTDKVPNTSATAASSGADLNGGAVKNACEQLRERLLRVAASQLGTNASDVRIVEGVARSLGSDQELAWDDLVRTAYFQRVQLSAAGYYRTEGLHWDAKSFRGSPFKYFAIGAAATEVEVDGFTGAYRIRRVDIVHDVGDSLSPLIDIGQVEGGFVQGAGWLTLEDLRWDTGDGPNRGRLLTQAASTYKLPSFSEMPEEFNVTLLENATEEGAVFGSKAVGEPPLMLAFSVREALRQAAAAFGPRGTAVELASPATPEAVYWAIESARQGGTAGDGRTHGAAASDAVAVRTGVEALSGA

SEQ ID NO: 6 - protein, 1347 aa, C. capitata
MTTNGNSFIVPVEKESPLIFFVNGKKVIDPTPDPECTLLTYLREKLRLCGTKLGCGEGGCGACTVMLSRVDRATNSVKHLAVNACLMPVCAMHGCAVTTIEGIGSTRTRLHPVQERLAKAHGSQCGFCTPGIVMSMYALLRSMPLPSMKDLEVAFQGNLCRCTGYRPILEGYKTFTKEFSCGMGEKCCKLQSNGNDVEKNGDDKLFERSAFLPFDPSQEPIFPPELHLNSQFDAENLLFKGPRSTWYRPVELSDLLKLKSENPHGKIIVGNTEVGVEMKFKQFLYTVHINPIKVPELNEMQELEDSILFGSAVTLMDIEEYLRERIAKLPEHETRFFRCAVKMLHYFAGKQIRNVASLGGNIMTGSPISDMNPILTAACAKLKVCSLVEGRIETREVCMGPGFFTGYRKNTIQPHEVLVAIHFPKSKKDQHFVAFKQARRRDDDIAIVNAAVNVTFESNTNIVRQIYMAFGGMAPTTVMVPKTSQIMAKQKWNRVLVERVSESLCAELPLAPTAPGGMIAYRRSLVVSLFFKAYLAISQELVKSNVIEEDAIPEREQSGAAIFHTPILKSAQLFERVCVEQSTCDPIGRPKVHASAFKQATGEAIYCDDIPRHENELYLALVLSTKAHAKIVSVDESDALKQAGVHAFFSSKDITEYENKVGSVFHDEEVFASERVYCQGQVIGAIVADSQVLAQRAARLVHIKYEELTPVIITIEQAIKHKSYFPNYPQYIVQGDVATAFEEADHVYENSCRMGGQEHFYLETNACVATPRDSDEIELFCSTQNPTEVQKLVAHVLSVPCHRVVCRSKRLGGGFGGKESRSIILALPVALASYRLRRPVRCMLDRDEDMMTTGTRHPFLFKYKVGFTKEGLITACDIECYNNAGCSMDLSFSVLDRAMNHFENCYRIPNVKVAGWVCRTNLPSNTAFRGFGGPQGMFAAEHIVRDVARIVGKDYLDIMQMNFYKTGDYTHYNQKLENFPIEKCFTDCLNQSEFHKKRLAIEEFNKKNRWRKRGIALVPTKYGIAFGAMHLNQAGALINIYGDGSVLLSHGGVEIGQGLHTKMIQCCARALGIPTELIHIAETATDKVPNTSPTAASVGSDINGMAVLDACEKLNQRLKPIREANPKATWQECISKAYFDRISLSASGFYKMPDVGDDPKTNPNARTYNYFTNGVGVSVVEIDCLTGDHQVLSTDIVMDIGSSLNPAIDIGQIEGAFMQGYGLFVLEELIYSPQGALYSRGPGMYKLPGFADIPGEFNVSLLTGAPNPRAVYSSKAVGEPPLFIGSTVFFAIKQAIAAARAERGLSITFELDAPATAARIRMACQDEFTDLIEQPSPGTYTPWNVVP

SEQ ID NO: 7 - protein, 1347 aa, N. crassa; identical to SEQ ID NO: 6

SEQ ID NO: 8 - protein, 1273 aa, M. Hansupus
MSNMFEFRLNGATVRVDGVSPNTTLLDFLRNRGLTGTKQGCAEGDCGACTVALVDRDAQGNRCLRAFNACIALVPMVAGRELVTVEGVGSSEKPHPVQQAMVKHYGSQCGFCTPGFIVSMAEGYSRKDVCTPSSVADQLCGNLCRCTGYRPIRDAMMEALAERDADASPATAIPSAPLGGPAEPLSALHYEATGQTFLRPTSWKELLDLRARHPEAHLVAGATELGVDITKKARRFPFLISTEGVESLREVRREKDCWYVGGAASLVALEEALGDALPEVTKMLNVFASRQIRQRATLAGNLVTASPIGDMAPVLLALDARLVLGSVRGERTVALSEFFLAYRKTALQADEVVRHIVIPHPAVPERGQRLSDSFKVSKRRELDISIVAAGFRVELDAHGVVSLARLGYGGVAATPVRAVRAEAALTGQPWTRETVDQVLPVLAEEITPISDQRGSAEYRRGLVAGLFEKFFAGTYSPVLDAAPGFEKGDAQVPADAGRALRHESAMGHVTGSARYVDDLAQRQPMLEVWPVCAPHAHARILKRDPTAARKVPGVVRVLMAEDIPGTNDTGPIRHDEPLLADREVLFHGQIVALVVGESVEACRAGARAVEVEYEPLPAILTVEDAMAQGSYHTEPHVIRRGDVDAALASSPHRLSGTMAIGGQEHFYLETQAAFAERGDDGDITVVSSTQHPSEVQAIISHVLHLPRSRVVVKSPRMGGGFGGKETQGNSPAALVALASWHTGRPTRWMMDRDVDMVVTGKRHPFHAAYEVGFDDEGKLLALRVQLVSNGGWSLDLSESITDRALFHLDNAYYVPALTYTGRVAKTHLVSNTAFRGFGGPQGMLVTEEVLAHVARSVGVPADVVRERNLYRGTGETNTTHYGQELEDERIHRVWEELKRTSDFEQRRAEVDAFNARSPFIKRGLAITPMKFGISFTATFLNQAGALVHLYRDGSVMVSHGGTEMGQGLHTKVQGVAMRELGVEASAVRIAKTATDKVPNTSATAASSGSDLNGAAVRLACITLRERLAPVAVRLLADRHGRTVAPEALLFSEGKVGLRGEPEVSLPFANVVEAAYLARVGLSATGYYQTPGIGYDKAKGRGRPFLYFAYGASVCEVEVDGHTGVKRVLRVDLLEDVGDSLNPGVDRGQIEGGFVQGLGWLTGEELRWDANGRLLTHSASTYAVPAFSDAPIDFRVRLLERAHQHNTIHGSKAVGEPPLMLAMSAREALRDAVGAFGQAGGGVALASPATHEALFLAIQKRLSRGAREDGREAA

SEQ ID NO: 9 - protein, 1367 aa, Artificial Sequence
  Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 10 - protein, 732 aa, E. cloacae
MKFDKPATTNPIDTLRVVGQPHTRIDGPRKTTGSAHYAYEWHDIAPNAAYGHVVGAPIAKGRITAIDTKAAEAAPGVLAVITADNAGPLGKGEKNTATLLGGPEIEHYHQAVALVVAETFEQARAAAALVKVTCKRAQGAYDLAAEKASVTEPPEDTPDKNVGDVATAFASAAVKLDAIYTTPDQSHMAMEPHASMAVWEGDNVTVWTSNQMIDWCRTDLALTLKIPPENVRIVSPYIGGGFGGKLFLRSDALLAALGARAVKRPVKVMLPRPTIPNNTTHRPATLQHIRIGTDTEGKIVAIAHDSWSGNLPGGTPETAVQQTELLYAGANRHTGLRLATLDLPEGNAMRAPGEAPGLMALEIAIDEIADKAGVDPVAFRILNDTQVDPANPERRFSRRQLVECLQTGAERFGWQKRHAQPGQVRDGRWLVGMGMAAGFRNNLVATSGARVHLNADGSVAVETDMTDIGTGSYTIIAQTAAEMLGLPLEKVDVRLGDSRFPVSAGSGGQWGANTSTAGVYAACVKLREAIARQLGFDPATAEFADETISAQGRSAPLAEAAKSGVLTAEDSIEFGDLDKEYQQSTFAGHFVEVGVDSATGEVRVRRMLAVCAAGRILNPITARSQVIGAMTMGLGAALMEELAVDTRLGYFVNHDMAAYEVPVHADIPEQEVIFLEDTDPISSPMKAKGVGELGLCGVSAAIANAIYNATGVRVRDYPITLDKLIDALPDAV

SEQ ID NO: 11 - protein, 32 aa, Artificial Sequence
  Synthetic polypeptide
SGGSSGGSSGSETPGTSESATPESSGGSSGGS

SEQ ID NO: 12 - protein, 10 aa, Artificial Sequence
  Synthetic polypeptide
SGGSGGSGGS

SEQ ID NO: 13 - protein, 17 aa, Artificial Sequence
  Synthetic polypeptide
KRTADGSEFEPKKKRKV

SEQ ID NO: 14 - protein, 4 aa, Artificial Sequence
  Synthetic polypeptide
SGGS

SEQ ID NO: 15 - protein, 623 aa, S. snoursei
MSHDPVPHLPPAAPLPHPLGAPSVRREGREKVTGAARYAAEHTPPGCAYAWPVPATVARGRITELDTAAALALPGVIAVLTHENAPRLASTGDPTLAVLQEDRVPHRGWYVALAVADTLEAARDAAEAVHVGYATEPHDVRITADHPRLYVPEEVFGGPGARERGDFDAAFAAAPATVDVAYTVPPLHNHPMEPHAATAQWTDGHLTVHDSSQGATRVCEDLAALFKLGTDEITVVSEHVGGGFGAKGTPRPQVVLAAMAARHTGRPVKLALPRRQLPGVVGHRAPTLHRVRIGAGHDGVITALAHEIVTHTSTVTEFVEQAAIPARMMYTSPHSRTVHRLAALDVPTPSWMRAPGEAPGMYALESALDELAVVLDIDPVELRIRNDPATEPDTGRPFSSRHLVECLRAGAERFGWLPRDPRPAVRRRGDLLLGTGVAAATYPVQISETEAEAHAAADGGYRIRVNATDIGTGARTVLTQIAAAVLGAPEDRVRVDIGSSDLPPAVLAGGSTGTASWGWAVHKACTSLLARLRAHHGPLPAEGIMAELSEWAPMALRAWRIISGLGLPTKYGSTPVALVMRAATEPVAGSGPSVEGPVSSGLVAMKRAPFSMSRMALVSASKL

SEQ ID NO: 16 - protein, 723 aa, S. albulus
MTPPPTTRTRAMSHPPEEAPFPPGPPPHPLGDPLVRREGREKVTGTARYAAEHTPDGCAYAWPVPATVVRGRITELDTGAALALPGVIAVLTHENAPRLAPTGDPTLALLQEDRVPHRGWYVALAVADTLEAARDAAEAVHVSYATEPHDVTLTADHPRLYVPAEVFGGPGARERGDFDTAFAAAPATVDVTYTVPPLHNHPMEPHAATALWTHGHLTVHDSSQGATRVREDLAALFKLGQDQITVHSEHVGGGFGSKGTPRPQVVLAAMAARHTGRPVKLALPRRHLPAVVGHRAPTLHRVRLGAGPDGVITALAHEIVTHTSTVAEFVEQAAMPARIMYTSPHSRTVHRLAALDVPTPSWMRAPGEAPGMYALESAVDELAVVLDLDPIDLRIRNEPGTEPDTGRPFSSRHLVDCLRAGAARFGWSSRDPRPAVRRQGDLLLGTGVAAATYPVQISATDAEAHAAADGTFRVRVNATDIGTGARTVLAQIAAAALGAPADRVRVEIGSSDLPPAVLAGGSTGTASWGWAVHKACTVLLARLREHRGPLPAEGVTVTEDTRRETEQPSPYSRHAFGAVFAEVQVDTRTGEVRARRLLGQYAAGHILNPRTARSQFVGGMVMGLGMALTEDSALDPVYGDFTARDLAAYHVPACADVPAIEAHWLDEEDPHLNPMGSKGIGEIGIVGTPAAIGNAVWHATGVRLRDLPLTPDRILTARTVPLT

SEQ ID NO: 17 - protein, 710 aa, S. himastatinicus
MTRVDGLDKVTGAATYAYEFPTPDVGYVWPVQATIARGRVTEVDGAPALARPGVLAVLDSGNAPRLNTEAQAGPDLFVLQSPEVAYHGQIVAAVVATSLEAAREGAAAVRVSYEQEPHDVVLRFDDERAQVAETVTDGSPGFVEHGDAEGALAAAPVRTEAMYTTPVEHTSPMEPHATIAAWDEDRLTLYNADQGPFMSSQLLAAVFGLDQGAVEVVAEYIGGGFGSKGIPRSPAVLAALAAKHLGRPVKIALTRQQMFQLIPYRAPTIQRIRLGAERDGRLTAIDHEVVQQRSAMAEFADQTGSSTRVMYAAPNIRTTVKTAPLDVLTPAWFRAPGHTPGMFALESAMDELATELEIDPVELRIRNDTGVDPDSGKPFSSRGLVACLREGAARFDWALRDPKPGIRREGRWLVGTGVASAHHPDYVFPSSATARAEADGTFTVRVGAVDIGTGGRTALTQLAADALGIPVERLRLEIGRASLGPAPFAGGSLGTASWGWAVDKACRALLAELDTYGGAVPDGGLEVRADTTEDVELRASFSRHSFGAHFAQVRVDTDTGEIRVDRMLGVFAAGRIVNPKTARSQFVGAMTMGLSMALLEIGEVDPVFGDFANHDFAGYHVAANADVPKLEALWLDEQDDNPNPVRGKGIGELGIVGAAAAVTNAFHHATGQRVRDLPIRVERSREALRAARAEAQKRGPGAAEQGKPVG

SEQ ID NO: 18 - protein, 806 aa, S. lividans; identical to SEQ ID NO: 5

SEQ ID NO: 19 - protein, 494 aa, Homo sapiens
MLASGMLLVALLVCLTVMVLMSVWQQRKSKGKLPPGPTPLPFIGNYLQLNTEQMYNSLMKISERYGPVFTIHLGPRRVVVLCGHDAVREALVDQAEEFSGRGEQATFDWVFKGYGVVFSNGERAKQLRRFSIATLRDFGVGKRGIEERIQEEAGFLIDALRGTGGANIDPTFFLSRTVSNVISSIVFGDRFDYKDKEFLSLLRMMLGIFQFTSTSTGQLYEMFSSVMKHLPGPQQQAFQLLQGLEDFIAKKVEHNQRTLDPNSPRDFIDSFLIRMQEEEKNPNTEFYLKNLVMTTLNLFIGGTETVSTTLRYGFLLLMKHPEVEAKVHEEIDRVIGKNRQPKFEDRAKMPYMEAVIHEIQRFGDVIPMSLARRVKKDTKFRDFFLPKGTEVFPMLGSVLRDPSFFSNPQDFNPQHFLNEKGQFKKSDAFVPFSIGKRNCFGEGLARMELFLFFTTVMQNFRLKSSQSPKDIDVSPKHVGFATIPRNYTMSFLPR

SEQ ID NO: 20 - DNA, 1480 bp, Homo sapiens
mctggcgagcggtatgctgctggttgcgctgctggtgtgcctgaccgtgatggttctgatgagcgtgtggcaacaacgtaaaagcaagggtaaactgccgccgggtccgaccccgctgccgtttatcggtaactacctgcaactgaacaccgaacagatgtataacagcctgatgaagatcagcgagcgttacggtccggttttcaccattcacctgggtccgcgtcgtgtggttgtgctgtgcggtcatgatgcggttcgtgaggcgctggttgaccaagcggaggaatttagcggtcgtggcgagcaggcgaccttcgattgggtttttaagggttatggcgttgtgttcagcaacggtgaacgtgcgaaacaactgcgtcgtttcagcatcgcgaccctgcgtgactttggtgtgggcaaacgtggcatcgaggaacgtatccaggaagaggcgggtttcctgattgatgcgctgcgtggcaccggtggcgcgaacattgacccgaccttctttctgagccgtaccgttagcaacgtgatcagcagcattgtgttcggtgaccgttttgattacaaggacaaagaatttctgagcctgctgcgtatgatgctgggtatcttccaatttaccagcaccagcaccggccagctgtatgagatgttcagcagcgttatgaagcacctgccgggtccgcagcaacaggcgttccaactgctgcagggcctggaagattttattgcgaagaaagtggagcacaaccaacgtaccctggacccgaacagcccgcgtgatttcatcgacagctttctgattcgtatgcaggaagaggagaagaacccgaacaccgaattttacctgaaaaacctggttatgaccaccctgaacctgttcatcggtggcaccgagaccgtgagcaccaccctgcgttatggtttcctgctgctgatgaagcacccggaagttgaggcgaaagtgcacgaagagatcgatcgtgttattggcaagaaccgtcaaccgaaatttgaggaccgtgcgaaaatgccgtacatggaagcggtgatccacgagattcagcgtttcggtgatgttattccgatgagcctggcgcgtcgtgtgaagaaagataccaagtttcgtgacttctttctgccgaaaggcaccgaggtgttcccgatgctgggcagcgtgctgcgtgatccgagcttctttagcaacccgcaagacttcaacccgcagcactttctgaacgagaagggccagttcaagaaaagcgatgcgttcgttccgtttagcatcggcaaacgtaactgcttcggtgaaggcctggcgcgtatggagctgtttctgttctttaccaccgttatgcaaaacttccgtctgaagagcagccagagcccgaaagacattgatgtgagcccgaaacacgttggctttgcgaccattccgcgtaactacaccatgagcttcctgccacgt

SEQ ID NO: 21 - DNA, 6407 bp, Artificial Sequence
  Synthetic polynucleotide
aacgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacatgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 22 - protein, 410 aa, Artificial Sequence
  Synthetic polypeptide
MIDMLVLRLPFIDSLVCSRLSGNDLIAFVDLSKIATLSGMNLSARTVEYHIDGDLTVSGLSHPFESLPTHYSGIAFKIYEGSKNFYPCVEIKASPAKVLQGHNVFGTTDLALCSEALLLNFANSLPCLYDLLDVNATTISRIDATFSARAPNENIAKQVIDHLRNVSNGQTKSTRSQNWESTVTWNETSRHRTLVAYLKHVELQHQIQQLSSKPSAKMTSYQKEQLKVLSNPDLLEFASGLVRFEARIKTRYLKSFGLPLNLFDAIRFASDYNSQGKDLIFDLWSFSFSELFKAFEGDSMNIYDDSAVLDAIQSKHFTITPSGKTSFAKASRYFGFYRRLVNEGYDSVALTMPRNSFWRYVSALVECGIPKSQLMNLSTCNNVVPLVRFINVDFSSQRPDWYNEPVLKIA

SEQ ID NO: 23 - protein, 111 aa, Artificial Sequence
  Synthetic polypeptide
MNIYDDSAVLDAIQSKHFTITPSGKTSFAKASRYFGFYRRLVNEGYDSVALTMPRNSFWRYVSALVECGIPKSQLMNLSTCNNVVPLVRFINVDFSSQRPDWYNEPVLKIA

SEQ ID NO: 24 - protein, 87 aa, Artificial Sequence
  Synthetic polypeptide
MIKVEIKPSQAQFTTRSGVSRQGKPYSLNEQLCYVDLGNEYPVLVKITLDEGQPAYAPGLYTVHLSSFKVGQFGSLMIDRLRLVPAK

SEQ ID NO: 25 - protein, 33 aa, Artificial Sequence
  Synthetic polypeptide
MEQVADFDTIYQAMIQISVVLCFALGIIAGGQR

SEQ ID NO: 26 - protein, 32 aa, Artificial Sequence
  Synthetic polypeptide
MSVLVYSFASFVLGWCLRSGITYFTRLMETSS

SEQ ID NO: 27 - protein, 73 aa, Artificial Sequence
  Synthetic polypeptide
MKKSLVLKASVAVATLVPMLSFAAEGDDPAKAAFNSLQASATEYIGYAWAMVVVIVGATIGIKLFKKFTSKAS

SEQ ID NO: 28 - protein, 424 aa, Artificial Sequence
  Synthetic polypeptide
MKKLLFAIPLVVPFYSHSAETVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 29 - protein, 112 aa, Artificial Sequence
  Synthetic polypeptide
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDK

SEQ ID NO: 30 - protein, 348 aa, Artificial Sequence
  Synthetic polypeptide
MAVYFVTGKLGSGKTLVSVGKIQDKIVAGCKIATNLDLRLQNLPQVGRFAKTPRVLRIPDKPSISDLLAIGRGNDSYDENKNGLLVLDECGTWFNTRSWNDKERQPIIDWFLHARKLGWDIIFLVQDLSIVDKQARSALAEHVVYCRRLDRITLPFVGTLYSLITGSKMPLPKLHVGVVKYGDSQLSPTVERWLYTGKNLYNAYDTKQAFSSNYDSGVYSYLTPYLSHGRYFKPLNLGQKMKLTKIYLKKFSRVLCLAIGFASAFTYSYITQPKPEVKKVVSQTYDFDKFTIDSSQRLNLSYRYVFKDSKGKLINSDDLQKQGYSLTYIDLCTVSIKKGNSNEIVKCN

SEQ ID NO: 31 - protein, 426 aa, Artificial Sequence
  Synthetic polypeptide
MKLLNVINFVFLMFVSSSSFAQVIEMNNSPLRDFVTWYSKQSGESVIVSPDVKGTVTVYSSDVKPENLRNFFISVLRANNFDMVGSIPSIIQKYNPNNQDYIDELPSSDNQEYDDNSAPSGGFFVPQNDNVTQTFKINNVRAKDLIRVVELFVKSNTSKSSNVLSIDGSNLLVVSAPKDILDNLPQFLSTVDLPTDQILIEGLIFEVQQGDALDFSFAAGSQRGTVAGGVNTDRLTSVLSSAGGSFGIFNGDVLGLSVRALKTNSHSKILSVPRILTLSGQKGSISVGQNVPFITGRVTGESANVNNPFQTIERQNVGISMSVFPVAMAGGNIVLDITSKADSLSSSTQASDVITNQRSIATTVNLRDGQTLLLGGLTDYKNTSQDSGVPFLSKIPLIGLLFSSRSDSNEESTLYVLVKATIVRAL

SEQ ID NO: 32 - protein, 1367 aa, Artificial Sequence
  Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 33 - protein, 1367 aa, Artificial Sequence
  Synthetic polypeptide
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 34 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 35 - protein, 503 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 36 - protein, 2136 aa, Homo sapiens
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 37 - protein, 721 aa, Homo sapiens
MGSLPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 38 - protein, 2002 aa, Homo sapiens
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 39 - protein, 1660 aa, Homo sapiens
MDSGPVYHGDSRQLSASGVPVNGAREPAGPSLLGTGGPWRVDQKPDWEAAPGPAHTARLEDAHDLVAFSAVAEAVSSYGALSTRLYETFNREMSREAGNNSRGPRPGPEGCSAGSEDLDTLQTALALARHGMKPPNCNCDGPECPDYLEWLEGKIKSVVMEGGEERPRLPGPLPPGEAGLPAPSTRPLLSSEVPQISPQEGLPLSQSALSIAKEKNISLQTAIAIEALTQLSSALPQPSHSTPQASCPLPEALSPPAPFRSPQSYLRAPSWPVVPPEEHSSFAPDSSAFPPATPRTEFPEAWGTDTPPATPRSSWPMPRPSPDPMAELEQLLGSASDYIQSVFKRPEALPTKPKVKVEAPSSSPAPAPSPVLQREAPTPSSEPDTHQKAQTALQQHLHHKRSLFLEQVHDTSFPAPSEPSAPGWWPPPSSPVPRLPDRPPKEKKKKLPTPAGGPVGTEKAAPGIKPSVRKPIQIKKSRPREAQPLFPPVRQIVLEGLRSPASQEVQAHPPAPLPASQGSAVPLPPEPSLALFAPSPSRDSLLPPTQEMRSPSPMTALQPGSTGPLPPADDKLEELIRQFEAEFGDSFGLPGPPSVPIQDPENQQTCLPAPESPFATRSPKQIKIESSGAVTVLSTTCFHSEEGGQEATPTKAENPLTPTLSGFLESPLKYLDTPTKSLLDTPAKRAQAEFPTCDCVEQIVEKDEGPYYTHLGSGPTVASIRELMEERYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLAGDNPKEEEVLRKSFQDLATEVAPLYKRLAPQAYQNQVTNEEIAIDCRLGLKEGRPFAGVTACMDFCAHAHKDQHNLYNGCTVVCTLTKEDNRCVGKIPEDEQLHVLPLYKMANTDEFGSEENQNAKVGSGAIQVLTAFPREVRRLPEPAKSCRQRQLEARKAAAEKKKIQKEKLSTPEKIKQEALELAGITSDPGLSLKGGLSQQGLKPSLKVEPQNHFSSFKYSGNAVVESYSVLGNCRPSDPYSMNSVYSYHSYYAQPSLTSVNGFHSKYALPSFSYYGFPSSNPVFPSQFLGPGAWGHSGSSGSFEKKPDLHALHNSLSPAYGGAEFAELPSQAVPTDAHHPTPHHQQPAYPGPKEYLLPKAPLLHSVSRDPSPFAQSSNCYNRSIKQEPVDPLTQAEPVPRDAGKMGKTPLSEVSQNGGPSHLWGQYSGGPSMSPKRTNGVGGSWGVFSSGESPAIVPDKLSSFGASCLAPSHFTDGQWGLFPGEGQQAASHSGGRLRGKPWSPCKFGNSTSALAGPSLTEKPWALGAGDFNSALKGSPGFQDKLWNPMKGEEGRIPAAGASQLDRAWQSFGLPLGSSEKLFGALKSEEKLWDPFSLEEGPAEEPPSKGAVKEEKGGGGAEEEEEELWSDSEHNFLDENIGGVAVAPAHGSILIECARRELHATTPLKKPNRCHPTRISLVFYQHKNLNQPNHGLALWEAKMKQLAERARARQEEAARLGLGQQEAKLYGKKRKWGGTVVAEPQQKEKKGVVPTRQALAVPTDSAVTVSSYAYTKVTGPYSRWI

SEQ ID NO: 40 - protein, 216 aa, E. coli
MLDLFADAEPWQEPLAAGAVILRRFAFNAAEQLIRDINDVASQSPFRQMVTPGGYTMSVAMTNCGHLGWTTHRQGYLYSPIDPQTNKPWPAMPQSFHNLCQRAATAAGYPDFQPDACLINRYAPGAKLSLHQDKDEPDLRAPIVSVSLGLPAIFQFGGLKRNDPLKRLLLEHGDVVVWGGESRLFYHGIQPLKAGFHPLTIDCRYNLTFRQAGKKE

SEQ ID NO: 41 - protein, 170 aa, Homo sapiens
MEEKRRRARVQGAWAAPVKSQAIAQPATTAKSHLHQKPGQTWKNKEHHLSDREFVFKEPQQVVRRAPEPRVIEEGVYEISLSPTGVSRVCLYPGFVDVKEADWILEQLCQDVPWKQRTGIREDSILQLTFKKSAPVSGTATAPQSCWYERPSPPHIPGPAILTRTRLWAP

SEQ ID NO: 42 - protein, 887 aa, Natronobacterium gregoryi
MTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFL

SEQ ID NO: 43 - protein, 525 aa, E. coli
MTENIHKHRILILDFGSQYTQLVARRVRELGVYCELWAWDVTEAQIRDFNPSGIILSGGPESTTEENSPRAPQYVFEAGVPVFGVCYGMQTMAMQLGGHVEASNEREFGYAQVEVVNDSALVRGIEDALTADGKPLLDVWMSHGDKVTAIPSDFITVASTESCPFAIMANEEKRFYGVQFHPEVTHTRQGMRMLERFVRDICQCEALWTPAKIIDDAVARIREQVGDDKVILGLSGGVDSSVTAMLLHRAIGKNLTCVFVDNGLLRLNEAEQVLDMFGDHFGLNIVHVPAEDRFLSALAGENDPEAKRKIIGRVFVEVFDEEALKLEDVKWLAQGTIYPDVIESAASATGKAHVIKSHHNVGGLPKEMKMGLVEPLKELFKDEVRKIGLELGLPYDMLYRHPFPGPGLGVRVLGEVKKEYCDLLRRADAIFIEELRKADLYDKVSQAFTVFLPVRSVGVMGDGRKYDWVVSLRAVETIDFMTAHWAHLPYDFLGRVSNRIINEVNGISRVVYDISGKPPATIEWE

SEQ ID NO: 44 - protein, 349 aa, S. scirui
MNFNNKTKYGKIQEFLRSNNEPDYRIKQITNAIFKQRISRFEDMKVLPKLLREDLINNFGETVLNIKLLAEQNSEQVTKVLFEVSKNERVETVNMKYKAGWESFCISSQCGCNFGCKFCATGDIGLKKNLTVDEITDQVLYFHLLGHQIDSISFMGMGEALANRQVFDALDSFTDPNLFALSPRRLSISTIGIIPSIKKITQEYPQVNLTFSLHSPYSEERSKLMPINDRYPIDEVMNILDEHIRLTSRKVYIAYIMLPGVNDSLEHANEVVSLLKSRYKSGKLYHVNLIRYNPTISAPEMYGEANEGQVEAFYKVLKSAGIHVTIRSQFGIDIDAACGQLYGNYQNSQ

SEQ ID NO: 45 - protein, 1052 aa, Artificial Sequence
  Synthetic polypeptide
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 46 - protein, 392 aa, A. aeolicus
MEIVQEGIAKIIVPEIPKTVSSDMPVFYNPRMRVNRDLAVLGLEYLCKKLGRPVKVADPLSASGIRAIRFLLETSCVEKAYANDISSKAIEIMKENFKLNNIPEDRYEIHGMEANFFLRKEWGFGFDYVDLDPFGTPVPFIESVALSMKRGGILSLTATDTAPLSGTYPKTCMRRYMARPLRNEFKHEVGIRILIKKVIELAAQYDIAMIPIFAYSHLHYFKLFFVKERGVEKVDKLIEQFGYIQYCFNCMNREVVTDLYKFKEKCPHCGSKFHIGGPLWIGKLWDEEFTNFLYEEAQKREEIEKETKRILKLIKEESQLQTVGFYVLSKLAEKVKLPAQPPIRIAVKFFNGVRTHFVGDGFRTNLSFEEVMKKMEELKEKQKEFLEKKKQG

SEQ ID NO: 47 - protein, 570 aa, S. cerevisiae
MEGFFRIPLKRANLHGMLKAAISKIKANFTAYGAPRINIEDFNIVKEGKAEILFPKKETVFYNPIQQFNRDLSVTCIKAWDNLYGEECGQKRNNKKSKKKRCAETNDDSSKRQKMGNGSPKEAVGNSNRNEPYINILEALSATGLRAIRYAHEIPHVREVIANDLLPEAVESIKRNVEYNSVENIVKPNLDDANVLMYRNKATNNKFHVIDLDPYGTVTPFVDAAIQSIEEGGLMLVTCTDLSVLAGNGYPEKCFALYGGANMVSHESTHESALRLVLNLLKQTAAKYKKTVEPLLSLSIDFYVRVFVKVKTSPIEVKNVMSSTMTTYHCSRCGSYHNQPLGRISQREGRNNKTFTKYSVAQGPPVDTKCKFCEGTYHLAGPMYAGPLHNKEFIEEVLRINKEEHRDQDDTYGTRKRIEGMLSLAKNELSDSPFYFSPNHIASVIKLQVPPLKKVVAGLGSLGFECSLTHAQPSSLKTNAPWDAIWYVMQKCDDEKKDLSKMNPNTTGYKILSAMPGWLSGTVKSEYDSKLSFAPNEQSGNIEKLRKLKIVRYQENPTKNWGPKARPNTS

SEQ ID NO: 48 - protein, 659 aa, Homo sapiens
MQGSSLWLSLTFRSARVLSRARFFEWQSPGLPNTAAMENGTGPYGEERPREVQETTVTEGAAKIAFPSANEVFYNPVQEFNRDLTCAVITEFARIQLGAKGIQIKVPGEKDTQKVVVDLSEQEEEKVELKESENLASGDQPRTAAVGEICEEGLHVLEGLAASGLRSIRFALEVPGLRSVVANDASTRAVDLIRRNVQLNDVAHLVQPSQADARMLMYQHQRVSERFDVIDLDPYGSPATFLDAAVQAVSEGGLLCVTCTDMAVLAGNSGETCYSKYGAMALKSRACHEMALRIVLHSLDLRANCYQRFVVPLLSISADFYVRVFVRVFTGQAKVKASASKQALVFQCVGCGAFHLQRLGKASGVPSGRAKFSAACGPPVTPECEHCGQRHQLGGPMWAEPIHDLDFVGRVLEAVSANPGRFHTSERIRGVLSVITEELPDVPLYYTLDQLSSTIHCNTPSLLQLRSALLHADFRVSLSHACKNAVKTDAPASALWDIMRCWEKECPVKRERLSETSPAFRILSVEPRLQANFTIREDANPSSRQRGLKRFQANPEANWGPRPRARPGGKAADEAMEERRRLLQNKRKEPPEDVAQRAARLKTFPCKRFKEGTCQRGDQCCYSHSPPTPRVSADAAPDCPETSNQTPPGPGAAAGPGID

SEQ ID NO: 49 - protein, 269 aa, E. coli
MSFSCPLCHQPLSREKNSYICPQRHQFDMAKEGYVNLLPVQHKRSRDPGDSAEMMQARRAFLDAGHYQPLRDAIVAQLRERLDDKATAVLDIGCGEGYYTHAFADALPEITTFGLDVSKVAIKAAAKRYPQVTFCVASSHRLPFSDTSMDAIIRIYAPCKAEELARVVKPGGWVITATPGPRHLMELKGLIYNEVHLHAPHAEQLEGFTLQQSAELCYPMRLRGDEAVALLQMTPFAWRAKPEVWQTLAAKEVFDCQTDFNIHLWQRSY

SEQ ID NO: 50 - protein, 255 aa, E. coli
MWIGIISLFPEMFRAITDYGVTGRAVKNGLLSIQSWSPRDFTHDRHRTVDDRPYGGGPGMLMMVQPLRDAIHAAKAAAGEGAKVIYLSPQGRKLDQAGVSELATNQKLILVCGRYEGIDERVIQTEIDEEWSIGDYVLSGGELPAMTLIDSVSRFIPGVLGHEASATEDSFAEGLLDCPHYTRPEVLEGMEVPPVLLSGNHAEIRRWRLKQSLGRTWLRRPELLENLALTEEQARLLAEFKTEHAQQQHKHDGMA

SEQ ID NO: 51 - protein, 339 aa, Homo sapiens
MSSEMLPAFIETSNVDKKQGINEDQEESQKPRLGEGCEPISKRQMKKLIKQKQWEEQRELRKQKRKEKRKRKKLERQCQMEPNSDGHDRKRVRRDVVHSTLRLIIDCSFDHLMVLKDIKKLHKQIQRCYAENRRALHPVQFYLTSHGGQLKKNMDENDKGWVNWKDIHIKPEHYSELIKKEDLIYLTSDSPNILKELDESKAYVIGGLVDHNHHKGLTYKQASDYGINHAQLPLGNFVKMNSRKVLAVNHVFEIILEYLETRDWQEAFFTILPQRKGAVPTDKACESASHDNQSVRMEEGGSDSDSSEEEYSRNELDSPHEEKQDKENHTESTVNSLPH

SEQ ID NO: 52 - protein, 336 aa, M. Jannaschii
MPLCLKINKKHGEQTRRILIENNLLNKDYKITSEGNYLYLPIKDVDEDILKSILNIEFELVDKELEEKKIIKKPSFREIISKKYRKEIDEGLISLSYDVVGDLVILQISDEVDEKIRKEIGELAYKLIPCKGVFRRKSEVKGEFRVRELEHLAGENRTLTIHKENGYRLWVDIAKVYFSPRLGGERARIMKKVSLNDVVVDMFAGVGPFSIACKNAKKIYAIDINPHAIELLKKNIKLNKLEHKIIPILSDVREVDVKGNRVIMNLPKFAHKFIDKALDIVEEGGVIHYYTIGKDFDKAIKLFEKKCDCEVLEKRIVKSYAPREYILALDFKINKK

SEQ ID NO: 53 - protein, 330 aa, P. Abyssi
MTLAVKVPLKEGEIVRRRLIELGALDNTYKIKREGNFLLIPVKFPVKGFEVVEAELEQVSRRPNSYREIVNVPQELRRFLPTSFDIIGNIAIIEIPEELKGYAKEIGRAIVEVHKNVKAVYMKGSKIEGEYRTRELIHIAGENITETIHRENGIRLKLDVAKVYFSPRLATERMRVFKMAQEGEVVFDMFAGVGPFSILLAKKAELVFACDINPWAIKYLEENIKLNKVNNVVPILGDSREIEVKADRIIMNLPKYAHEFLEHAISCINDGGVIHYYGFGPEGDPYGWHLERIRELANKFGVKVEVLGKRVIRNYAPRQYNIAIDFRVSF

SEQ ID NO: 54 - protein, 19 aa, Artificial Sequence
  Synthetic polypeptide
NLSKRPAAIKKAGQAKKKK

SEQ ID NO: 55 - protein, 9 aa, Artificial Sequence
  Synthetic polypeptide
PAAKRVKLD

SEQ ID NO: 56 - protein, 11 aa, Artificial Sequence
  Synthetic polypeptide
RQRRNELKRSF

SEQ ID NO: 57 - protein, 38 aa, Artificial Sequence
  Synthetic polypeptide
NQSSNFGPMKGGNFGGRSSGPYGGGGQYFAKPRNQGGY

SEQ ID NO: 58 - DNA, 23 bp, Artificial Sequence
  Synthetic polynucleotide
  misc_feature (1)..(8): n is a, c, g, t or u
  misc_feature (9)..(20): n is a, c, g, t or u
  misc_feature (21)..(21): n is a, c, g, t or u
nnnnnnnnnnnnnnnnnnnnngg

SEQ ID NO: 59 - DNA, 15 bp, Artificial Sequence
  Synthetic polynucleotide
  misc_feature (1)..(12): n is a, c, g, t or u
  misc_feature (13)..(13): n is a, c, g, t or u
nnnnnnnnnnnnngg

SEQ ID NO: 60 - DNA, 23 bp, Artificial Sequence
  Synthetic polynucleotide
  misc_feature (1)..(9): n is a, c, g, t or u
  misc_feature (10)..(20): n is a, c, g, t or u
  misc_feature (21)..(21): n is a, c, g, t or u
nnnnnnnnnnnnnnnnnnnnngg

SEQ ID NO: 61 - DNA, 14 bp, Artificial Sequence
  Synthetic polynucleotide
  misc_feature (1)..(11): n is a, c, g, t or u
  misc_feature (12)..(12): n is a, c, g, t or u
nnnnnnnnnnnngg

SEQ ID NO: 62 - DNA, 27 bp, Artificial Sequence
  Synthetic polynucleotide
  misc_feature (1)..(8): n is a, c, g, t or u
  misc_feature (9)..(20): n is a, c, g, t or u
  misc_feature (21)..(22): n is a, c, g, t or u
  misc_feature (27)..(27): w is a or t
nnnnnnnnnnnnnnnnnnnnnnagaaw

SEQ ID NO: 63 - DNA, 19 bp, Artificial Sequence
  Synthetic polynucleotide
  misc_feature (1)..(12): n is a, c, g, t or u
  misc_feature (13)..(14): n is a, c, g, t or u
  misc_feature (19)..(19): w is a or t
nnnnnnnnnnnnnnagaaw

SEQ ID NO: 64 - DNA, 27 bp, Artificial Sequence
  Synthetic polynucleotide
  misc_feature (1)..(9): n is a, c, g, t or u
  misc_feature (10)..(20): n is a, c, g, t or u
  misc_feature (21)..(22): n is a, c, g, t or u
  misc_feature (27)..(27): w is a or t
nnnnnnnnnnnnnnnnnnnnnnagaaw

SEQ ID NO: 65 - DNA, 18 bp, Artificial Sequence
  Synthetic polynucleotide
  misc_feature (1)..(11): n is a, c, g, t or u
  misc_feature (12)..(13): n is a, c, g, t or u
  misc_feature (18)..(18): w is a or t
nnnnnnnnnnnnnagaaw

SEQ ID NO: 66 - DNA, 25 bp, Artificial Sequence
  Synthetic polynucleotide
  misc_feature (1)..(8): n is a, c, g, t or u
  misc_feature (9)..(20): n is a, c, g, t or u
  misc_feature (21)..(21): n is a, c, g, t or u
  misc_feature (24)..(24): n is a, c, g, t or u
nnnnnnnnnnnnnnnnnnnnnggng

SEQ ID NO: 67 - DNA, 17 bp, Artificial Sequence
  Synthetic polynucleotide
  misc_feature (1)..(12): n is a, c, g, t or u
  misc_feature (13)..(13): n is a, c, g, t or u
  misc_feature (16)..(16): n is a, c, g, t or u
nnnnnnnnnnnnnggng

SEQ ID NO: 68 - DNA, 25 bp, Artificial Sequence
  Synthetic polynucleotide
  misc_feature (1)..(9): n is a, c, g, t or u
  misc_feature (10)..(20): n is a, c, g, t or u
  misc_feature (21)..(21): n is a, c, g, t or u
  misc_feature (24)..(24): n is a, c, g, t or u
nnnnnnnnnnnnnnnnnnnnnggng

SEQ ID NO: 69 - DNA, 16 bp, Artificial Sequence
  Synthetic polynucleotide
  misc_feature (1)..(11): n is a, c, g, t or u
  misc_feature (12)..(12): n is a, c, g, t or u
  misc_feature (15)..(15): n is a, c, g, t or u
nnnnnnnnnnnnggng

SEQ ID NO: 70-71 - skipped (2 entries)

SEQ ID NO: 72 - DNA, 90 bp, Artificial Sequence
  Synthetic polynucleotide
tgagattagagatatagagtaagatgatggtgtgaaatggtaagcgtatgatgaagtagttaagtttgtagtgggttggtaaattagtag

SEQ ID NO: 73 - DNA, 90 bp, Artificial Sequence
  Synthetic polynucleotide
actctaatctctatatctcattctactaccacactttaccattcgcatactacttcatcaattcaaacatcacccaaccatttaatcatc

SEQ ID NO: 74 - protein, 30 aa, Artificial Sequence
  Synthetic polypeptide
SILSISYSSPTFHYAYSSTTLNTTPQYILL

SEQ ID NO: 75 - DNA, 13 bp, Artificial Sequence
  Synthetic polynucleotide
ggtaagcgtatga

SEQ ID NO: 76 - DNA, 40 bp, Artificial Sequence
  Synthetic polynucleotide
acttcatcatacgcttaccatttcacaccatcatcttact

SEQ ID NO: 77 - DNA, 40 bp, Artificial Sequence; identical to SEQ ID NO: 76
  Synthetic polynucleotide

SEQ ID NO: 78 - DNA, 40 bp, Artificial Sequence
  Synthetic polynucleotide
acttcatcatactcttaccatttcacaccatcatcttact

SEQ ID NO: 79 - DNA, 39 bp, Artificial Sequence
  Synthetic polynucleotide
acttcatcataccttaccatttcacaccatcatcttact

SEQ ID NO: 80 - DNA, 35 bp, Artificial Sequence
  Synthetic polynucleotide
acttcatcatacccatttcacaccatcatcttact

SEQ ID NO: 81 - protein, 7 aa, Artificial Sequence
  Synthetic polypeptide
PKKKRKV

SEQ ID NO: 82 - protein, 30 aa, Artificial Sequence
  Synthetic polypeptide
MDSLLMNRRKFLYQFKNVRWAKGRRETYLC

SEQ ID NO: 83 - protein, 24 aa, Artificial Sequence
  Synthetic polypeptide
  misc_feature (4)..(24): may be absent
GGSGGSGGSGGSGGSGGSGGSGGS

SEQ ID NO: 84 - protein, 18 aa, Artificial Sequence
  Synthetic polypeptide
KRTADGSEFESPKKKRKV

SEQ ID NO: 85 - protein, 16 aa, Artificial Sequence
  Synthetic polypeptide
  misc_feature (3)..(12): Xaa can be any naturally occurring amino acid
KRXXXXXXXXXXKKKL

SEQ ID NO: 86 - DNA, 125 bp, Artificial Sequence
  Synthetic polynucleotide
  misc_feature (1)..(8): n is a, c, g, t or u
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 87 - DNA, 121 bp, Artificial Sequence
  Synthetic polynucleotide
  misc_feature (1)..(18): n is a, c, g, t or u
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 88 - DNA, 109 bp, Artificial Sequence
  Synthetic polynucleotide
  misc_feature (1)..(20): n is a, c, g, t or u
nnnnnnnnnnnnnnnnnnnngtttttgtactctcagaaatgcagaagctacaaagataaggcttcatgccgaaatcaacaccctgtcattttatggcagggtgtttttt

SEQ ID NO: 89 - DNA, 102 bp, Artificial Sequence
  Synthetic polynucleotide
  misc_feature (1)..(20): n is a, c, g, t or u
nnnnnnnnnnnnnnnnnnnngttttagagctagaaatagcaagttaaaataaggctagtccgttatcaacttgaaaaagtggcaccgagtcggtgctttttt

SEQ ID NO: 90 - DNA, 88 bp, Artificial Sequence
  Synthetic polynucleotide
  misc_feature (1)..(20): n is a, c, g, t or u
nnnnnnnnnnnnnnnnnnnngttttagagctagaaatagcaagttaaaataaggctagtccgttatcaacttgaaaaagtgttttttt

SEQ ID NO: 91 - DNA, 76 bp, Artificial Sequence
  Synthetic polynucleotide
  misc_feature (1)..(20): n is a, c, g, t or u
nnnnnnnnnnnnnnnnnnnngttttagagctagaaatagcaagttaaaataaggctagtccgttatcatttttttt

SEQ ID NO: 92 - RNA, 81 nt, Artificial Sequence
  Synthetic polynucleotide
  misc_feature (1)..(1): may be modified by a guide sequence
guuuuagagcuagaaauagcaaguuaaaauaaggcuaguccguuaucaacuugaaaaaguggcaccgagucggugcuuuuu

SEQ ID NO: 93 - protein, 155 aa, Artificial Sequence
  Synthetic polypeptide
  misc_feature (6)..(155): may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 94 - protein, 31 aa, Artificial Sequence
  Synthetic polypeptide
  misc_feature (2)..(31): may be absent
GGGGGGGGGGGGGGGGGGGGGGGGGGGGGGG

SEQ ID NO: 95 - protein, 155 aa, Artificial Sequence
  Synthetic polypeptide
  misc_feature (6)..(155): may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 96 - protein, 93 aa, Artificial Sequence
  Synthetic polypeptide
  misc_feature (4)..(93): may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 97 - protein, 124 aa, Artificial Sequence
  Synthetic polypeptide
  misc_feature (5)..(124): may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 98 - protein, 62 aa, Artificial Sequence
  Synthetic polypeptide
  misc_feature (1)..(1): Xaa can be any naturally occurring amino acid
  misc_feature (3)..(62): may be absent
XPXPXPXPXPXPXPXPXPXPXPXPXPXPXPXPXPXPXPXPXPXPXPXPXPXPXPXPXPXPXP

SEQ ID NO: 99 - protein, 16 aa, Artificial Sequence
  Synthetic polypeptide
SGSETPGTSESATPES

SEQ ID NO: 100 - protein, 1082 aa, Artificial Sequence
  Synthetic polypeptide
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL